(19) United States
(12) Patent Application Publication
(10) Pub. No.: US 2025/0002554 A1
(43) Pub. Date: Jan. 2, 2025

(54) DELTA PROTOCADHERIN THERAPIES

(71) Applicant: CORNELL UNIVERSITY, Ithaca, NY (US)

(72) Inventor: David Lin, Ithaca, NY (US)

(21) Appl. No.: 18/708,363

(22) PCT Filed: Nov. 8, 2022
(86) PCT No.: PCT/US2022/079485

§ 371 Date: May 8, 2024

Related U.S. Application Data
(60) Provisional application No. 63/277,044, filed on Nov. 8, 2021.

(51) Int. Cl. C07K 14/705; A61K 9/50; A61K 38/00; A61K 45/06; A61P 25/28

(52) U.S. Cl. CPC C07K 14/705; A61K 9/5068; A61K 45/06; A61P 25/28; A61K 38/00

(57) ABSTRACT

There is an essential need to identify and develop new compounds and/or agents that can be used to enhance neuronal regrowth. Described in several embodiments herein are delta protocadherin compositions that can include a delta protocadherin gene or gene product. Also described herein are methods of making and using the same, particularly for treatment of a neurologic and/or neurovegetative disease, disorder, condition, nerve injury, and/or a symptom thereof.

### CROSS-REFERENCE TO RELATED APPLICATIONS

This application claims priority to and the benefit of U.S. Provisional Patent Application No. 63/277,044, filed Nov. 8, 2021, the contents of the above-identified application are hereby fully incorporated herein by reference in their entirety.

### SEQUENCE LISTING

This application contains a sequence listing filed in electronic form as an xml file entitled CORNL-0735WP_ST26.xml, created on Nov. 8, 2022 and having a size of 21,446 bytes. The content of the sequence listing is incorporated herein in its entirety.

### TECHNICAL FIELD

The subject matter disclosed herein is generally directed to delta protocadherin replacement therapies and modification therapies for treatment and/or prevention of a disease, disorder, condition and/or a symptom thereof.

### BACKGROUND

Spinal cord injuries, traumatic damage to peripheral neurons, or degeneration caused by neurodegenerative disorders can have significant lifelong implications. Understanding the mechanisms that can promote the repair of the nervous system has major clinical implications for human health.

Numerous environmental and genetic factors have been identified that can influence nervous system repair (Curcio M, Bradke F. Axon Regeneration in the Central Nervous System: Facing the Challenges from the Inside. Annu. Rev. Cell Dev. Biol. 2018 Oct. 6; 34:495-521 and Fawcett J W. The Struggle to Make CNS Axons Regenerate: Why Has It Been so Difficult? Neurochem Res. Springer US; 2020 Jan. 1; 45(1):144-58). Despite this, treatment options for repair are still highly limited. Current efforts span a variety of different approaches. These include using different cell-types to replace or encourage growth (e.g., stem cells, neural progenitors, or glial cells (Ahuja C S, Mothe A, Khazaei M, Badhiwala J H, Gilbert E A, van der Kooy D, et al. The leading edge: Emerging neuroprotective and neuroregenerative cell-based therapies for spinal cord injury. Stem Cells Transl Med. 2020 December; 9(12):1509-30. PMCID: PMC7695641), developing new conduits to promote regrowth (Carvalho C R, Oliveira J M, Reis R L. Modern Trends for Peripheral Nerve Repair and Regeneration: Beyond the Hollow Nerve Guidance Conduit. Front Bioeng Biotechnol. 2019; 7:337. PMCID: PMC6882937), identifying approaches to activate axon guidance pathways (Hilton B J, Bradke F. Can injured adult CNS axons regenerate by recapitulating development? Development. 2017 Oct. 1; 144(19):3417-29), treating individuals with drugs to reduce inflammation (Ren Y, Young W. Managing inflammation after spinal cord injury through manipulation of macrophage function. Neural Plast. 2013; 2013:945034. PMCID: PMC3833318), and adding purified proteins known to promote axonal sprouting and growth (Wu Q, Xiang Z, Ying Y, Huang Z, Tu Y, Chen M, et al. Nerve growth factor (NGF) with hypoxia response elements loaded by adeno-associated virus (AAV) combined with neural stem cells improve the spinal cord injury recovery. Cell Death Discov. 2021 Oct. 21; 7(1):301. PMCID: PMC8531363).

However, none of these treatments alone is typically successful. No one single protein, device, or treatment can overcome the many varied factors that can prevent proper regeneration. It is therefore essential to identify and develop new compounds and/or agents that can be used to further enhance regrowth.

Citation or identification of any document in this application is not an admission that such a document is available as prior art to the present invention.

### SUMMARY

Described in certain example embodiments herein are compositions comprising a delta protocadherin gene or gene product, a delta protocadherin modifier, or both. In certain example embodiments, the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof. In certain example embodiments, the delta protocadherin modifier is effective to modify a delta protocadherin gene or gene product, optionally where the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof.

In certain example embodiments, the delta protocadherin modifier is effective to increase or decrease expression and/or activity of the one or more delta protocadherin genes or gene products. In certain example embodiments, wherein the delta protocadherin modifier is effective to modify the gene or gene product polynucleotide and/or polypeptide sequence. In certain example embodiments, the delta protocadherin modifier is effective to cause insertions and/or deletions in the delta protocadherin gene.

In certain example embodiments, the delta protocadherin modifier comprises a genetic modification system (e.g., a CRISPR-Cas system, a zinc finger nuclease system, a meganuclease system), an RNA modification system (e.g., an RNAi system, an RNA-editing system (e.g., an ADAR or CRISPR-Cas-ADAR based system), a CRISPRi system) an antibody or fragment thereof, an aptamer, or any combination thereof.

In certain example embodiments, the delta protocadherin gene or gene product comprises a delta protocadherin encoding polynucleotide (e.g., DNA or RNA) or fragment thereof, a delta protocadherin polypeptide or functional fragment thereof, or any combination thereof.

In certain example embodiments, the composition comprises a delta protocadherin gene or gene product that is a functional delta protocadherin gene or gene product and a delta protocadherin modifier that inhibits a non-functional or aberrant delta protocadherin gene or gene product.

In certain example embodiments, the delta protocadherin gene or gene product, the delta protocadherin modifier, or both are contained in a vesicle, optionally an exosome or microvesicle. In certain example embodiments, the extracellular vesicles are olfactory derived extracellular vesicles, optionally olfactory sensory neuron derived extracellular vesicles. In certain example embodiments, the delta protocadherin gene or gene product, the delta protocadherin modifier, or both are native to the extracellular vesicle or exogenous to the extracellular vesicle.

In certain example embodiments, the composition further includes a cargo, wherein the cargo is optionally a polynucleotide, a polypeptide, a nutrient (e.g., lipid, amino acid, carbohydrate, peptide, protein, sugar, vitamin, mineral, and/or the like), genetic modifying system or component thereof, antibody or fragment thereof, aptamer, affibody, small molecule chemical agent (e.g., a therapeutic and/or prevention), an immunomodulator, a hormone, an antipyretic, an anxiolytic, an antipsychotic, an analgesic, an antispasmodic, an anti-inflammatory agent, an anti-epileptic, an anti-histamine, an anti-infective, a radiation sensitizer, a chemotherapeutic, or any combination thereof.

In certain example embodiments, the composition comprises one or more targeting moieties coupled to and/or otherwise associated with the delta protocadherin gene or gene product, the delta protocadherin modifier, or both, wherein the targeting moiety is optionally a peptide, polypeptide, polynucleotide, sugar, a chemical molecule, a polymer, a lipid, a glycan, a peptidoglycan, or any combination or complex thereof (e.g., receptors, receptor ligands, antibodies and fragments thereof, aptamers, affibodies, antibody and/or aptamer epitopes, binding agents and their binding partners (e.g., biotin and streptavidin, enzymes and their substrates, a targeting nucleic acid, target nucleic acid and guided nuclease (e.g., miRNA, gRNA, RISC, Cas, etc.), guide nucleic acid for a guided nuclease system, and/or the like.

In certain example embodiments, the composition is frozen, dehydrated, lyophilized, or otherwise modified for storage.

The compositions can have one or more beneficial and/or therapeutic effects. In certain example embodiments, the composition is effective to stimulate axonal growth and/or increase the rate of axonal growth in a peripheral neuron, a central nervous system neuron, or both. In certain example embodiments, the composition the composition is effective to increase correct axonal connectivity during neuron regeneration.

Described in certain example embodiments herein are formulations comprising of any one of the preceding paragraphs and a pharmaceutically acceptable carrier or excipient.

In certain example embodiments, the formulation is adapted for oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, buccal, conjunctival, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradiscal, intraductal, intraduodenal, intradural, intraepidermal, intraesophageal, intragastric, intragingival, intraileal, intralesional, intraluminal, intralymphatic, intramedullary, intrameningeal, intraovarian, intrapericardial, intrapleural, intraprostatic, intrapulmonary, intrasinal, intraspinal, intrasynovial, intratendinous, intratesticular, subarachnoid, subconjunctival, subcutaneous, sublingual, submucosal, topical, transdermal, transmucosal, transplacental, transtracheal, transtympanic, or any combination thereof administration.

Described in certain example embodiments herein are methods of treating a disease, disorder, and/or condition in a subject in need thereof, the method comprising administering a composition or a formulation as in any one of the preceding paragraphs to the subject in need thereof.

Described in certain example embodiments herein are methods of increasing axonal growth and/or the rate of axonal growth during neuron development and/or regeneration, the method comprising administering a composition or a formulation as in any one of the preceding paragraphs to the subject in need thereof.

In certain example embodiments, the subject in need thereof has a nerve injury, nerve death, aberrant neuron connectivity, aberrant neuron activity, a neuropathy, or any combination thereof.

In certain example embodiments, the subject in need thereof has or is suspected of having a neurodegenerative disease, disorder, and/or condition.

In certain example embodiments, the subject in need thereof has, has had, or is suspected of having an epilepsy, a seizure disease, disorder or condition, or a disease, disorder, or condition in which seizures are a symptom or result of the disease, disorder, or condition, including but not limited to non-epileptic seizures.

In certain example embodiments, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In certain example embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof.

In certain example embodiments, the subject in need thereof has, has had, or is suspected of having a CNS neuron/nerve and/or a peripheral neuron/nerve injury, disease, disorder, and/or condition.

In certain example embodiments, the disease or disorder is a genetic disease, disorder, and/or condition.

In certain example embodiments, the disease or disorder is not a genetic disease, disorder, and/or condition.

Described in certain example embodiments herein are methods of promoting stem cell division or differentiation and/or cell reprogramming, comprising administering a delta protocadherin gene, gene product or modifier composition of the present disclosure or a formulation thereof to a stem cell or epithelial cell or population thereof.

In certain example embodiments, the cell is a differentiated cell. In certain example embodiments, the cell is an epithelial cell. In certain example embodiments, the cell is a neuron cell. In certain example embodiments, the stem cell is an induced pluripotent stem cell.

Described in certain example embodiments are methods of increasing neuron synapse formation, connectivity, or both during neuron development and/or regeneration, the method comprising administering a delta protocadherein gene, gene product or modifier composition of the present disclosure or a formulation thereof claims to the subject in need thereof.

In certain example embodiments, the subject in need thereof has a nerve injury, nerve death, aberrant neuron connectivity, aberrant neuron activity, a neuropathy, or any combination thereof.

In certain example embodiments, the subject in need thereof has or is suspected of having a neurodegenerative disease, disorder, and/or condition.

In certain example embodiments, the subject in need thereof has, has had, or is suspected of having an epilepsy, a seizure disease, disorder or condition, or a disease, disorder, or condition in which seizures are a symptom or result of the disease, disorder, or condition, including but not limited to non-epileptic seizures. In certain example embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In certain example embodiments, the subject in need thereof has, has had, or is suspected of having a dementia, a stroke, Alzheimer's disease, Motor neuron disease, Huntington's disease, Parkinson's disease, a Parkinsonism atrophy, corticobasal degeneration, diffuse Lewy body disease, spinal muscular atrophy, Friedreich ataxia, amyotrophic lateral sclerosis, or any combination thereof.

In certain example embodiments, the subject in need thereof has, has had, or is suspected of having a CNS neuron/nerve and/or a peripheral neuron/nerve injury, disease, disorder, and/or condition.

In certain example embodiments, the disease or disorder is a genetic disease, disorder, and/or condition.

In certain example embodiments, the disease or disorder is not a genetic disease, disorder, and/or condition.

Described in certain example embodiments herein are devices comprising a delta protocadherin gene, gene product or modifier composition of the present disclosure or a formulation thereof, wherein the composition is fixed in a pattern on one or more surfaces on the device.

In certain example embodiments, the composition is dried.

In certain example embodiments, the pattern is configured to direct correct neuron growth.

In certain example embodiments, the device is an implantable device.

Described in certain example embodiments herein are methods of treating a nerve or neurodegenerative injury, disease, disorder, and/or condition in a subject in need thereof, comprising implanting the device of the present disclosure into the subject in need thereof.

Described in certain example embodiments herein are methods of directing, increasing/enhancing axonal growth, the rate of axonal growth, synapse formation, connectivity, or any combination thereof during neuron development and/or regeneration, the method comprising implanting the device of the present disclosure into the subject in need thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Effect of neuron length per cell for dorsal root ganglions (DRGs) cultured without (FIG. 1A) or with (FIG. 1B) EVs derived from OSNs.

FIG. 2A-2G—Culturing primary olfactory neurons (FIG. 2A), purification of olfactory neuronal EVs (FIG. 2B) and their effect on OSNs from normal (FIG. 2C-2F) or OSNs from pcdh 19−/− mice (FIG. 2G).

FIG. 3—General structure of olfactory epithelium.

FIG. 4A-4B—Schematic of correct and incorrect glomeruli regeneration in the olfactory bulb after ablation by methimazole.

FIG. 5A-5B—Microscopic images demonstrating the olfactory epithelium after methimazole treatment at day 0 in saline control (FIG. 5A) and methimazole ablated (FIG. 5B) olfactory epithelium.

FIG. 6—OSN derived EVs promote olfactory epithelium regeneration.

FIG. 7—Microscopic (H &E stained) images of olfactory epithelium after methimazole ablation demonstrating sloughing followed by initial disorganized initial regeneration with no treatment with EVs.

FIG. 8A-8B—Microscopic (H &E stained) images of olfactory epithelium after control treated (saline) and OSN-derived EV treated olfactory epithelium.

FIG. 9—Fluorescent microscopic images of glomeruli at day 21 post ablation in saline treated, MI only (no control or EV treatment), or ablated+EV treated mice. EVs promoted better regrowth and neuron targeting to the olfactory bulb of olfactory neurons in vivo.

FIG. 10—General Nano LC MS/MS results.

FIG. 11—Map of Pcdh19 CRISPR mouse mutants of known nonsense mutations.

FIG. 12—Light sheet microscopy image of the olfactory bulb.

FIG. 13A-13B—Lateral view of glomeruli from wild-type Pcdh19 mice following regeneration.

FIG. 14A-14B—Medial view of glomeruli from wild-type Pcdh19 mice following regeneration.

FIG. 15A-15F—Male Pcdh19 mutants possess olfactory targeting defects following regeneration. (FIG. 15A-15B) Fluorescent imaging of olfactory glomeruli from wild-type and mutant Pcdh 19 mice. (FIG. 15C-15D) Graphs demonstrating targeting promiscuity in mutant mice (FIG. 15D) as compared to wild-type (FIG. 15C) and FIG. 15E-15F.

FIG. 16A-16F—Female Pcdh19 mutants possess olfactory targeting defects as compared to heterozygous females. (FIG. 16A-16B) Fluorescent imaging of olfactory glomeruli from wild-type and mutant Pcdh 19 mice. (FIG. 16C-16D) Graphs demonstrating targeting promiscuity in mutant female mice (FIG. 16D) as compared to heterozygous female mice (FIG. 16C) and FIG. 16E-16F.

FIG. 17A-17C—OSN derived EVs applied to cultured dorsal root ganglion (DRG) neurons increased neurite length.

FIG. 18A-18B—OSN derived EVs applied to transected sciatic nerves promoted improved axon organization during regeneration and may promote DRG regeneration.

FIG. 19A-19B—EV stock purification. EV stock collected from olfactory sensory neurons was successfully purified as shown by (+) flotillin (FIG. 19A) and (−) IkB detection (FIG. 19B).

FIG. 20—OSN layer condition comparative. The OSN layer for each condition's epithelium was measured at five randomized locations. Day 0 saline and MZ layer were significantly different (p<0.05), showing that MZ worked. Additionally, all EV conditions were significantly different from the day 14 saline condition (p<0.05).

FIG. 21A-21B—Day 0 imaging of olfactory epithelium. Both GFP imaging and hematoxylin and eosin (H&E) staining of the epithelium confirmed that methimazole (MZ) destroyed the olfactory sensory neuron (OSN) layer at day 0.

FIG. 22A-22D—H&E staining of the epithelium showed different cell patterns in the OSN layer within and across conditions. The MZ OSN layer appeared more unhealthy and loosely packed than the EV conditions. FIG. 22A. Methimazole only condition. FIG. 22B. 2 μg EV condition. FIG. 22C. 4 μg EV condition. FIG. 22D. 8 μg EV condition.

FIG. 23A-23E—Fluorescent microscopic imaging of OSN axons under different conditions at day 14. FIG. 23A. Saline condition. FIG. 23B. Methimazole only condition.

FIG. 23C. 2 μg EV condition. FIG. 23D. 4 μg EV condition. FIG. 23E. 8 μg EV condition. At day 14, all test condition treatments caused OSN axons to appear cloudy and not well defined as compared to saline treated OSN axons. At day 28, EV treated OSN axons had recovered more than untreated regenerating axons. 8 μg EV treated regenerating axons most closely resembled saline OSN axons.

FIG. 24A-24C—Experimental Apparatus: dissection and isolation of olfactory epithelium. FIG. 24A. Dorsal view of a neonatal mouse head. The line depicts the intended cut through the midsagittal plane. FIG. 24B. Schematic representation of mouse olfactory systems. FIG. 24C Sagittal view of an opened nasal cavity.

FIG. 25A-25C—Data analysis Apparatus: Immunostained sample images of neurons and ImageJ software for analysis of neurons. Immunostained images were analyzed (FIG. 25A) via ImageJ software, which traces neurons based on interpretation of where cell bodies and neurites are located. FIG. 25B shows the input and FIG. 25C shows the output, which ultimately converts into an excel sheet for statistical analysis.

FIG. 26A-26B—Western blot for verification of EV purification. FIG. 26A shows exposure of lysate and two EV samples to flotillin-2 primary antibody. FIG. 26B shows exposure of the lysate and one EV sample to IkB alpha primary antibody.

FIG. 27—Nanosight NS300 Nanoparticle Analysis for Secondary Verification of EV Purification. There are characteristic peaks (36 nm, 74 nm, and 128 nm) as well as smaller ones (183 nm, 372 nm, and 448 nm) that are all within expected ranges in a heterogenous population of EVs (both exosomes and microvesicles).

FIG. 28—Immunostained images of single concentration (8 ug) comparing EVD1 and EVD2 with Ctrl. Neurites are traced in red and cell bodies are stained with DAPI. Differences (arrows pointing out neurite extensions) can be observed between the two EV types with control with apparent greater growth of neurite lengths.

FIG. 29A-29B—Graphical Representation of Growth Distribution as a Measure of Total Length of Neurite Per Cell. At a selected concentration of 8 ug, both EVD1 and EVD2 showed significantly greater average total length of neurite compared to control (p<<<0.001); no significant differences were observed between the two EV populations (p=0.57) (FIG. 29A). To better visualize the distribution of data, a violin plot (FIG. 29B) (log of average total length of neurite) was made and a bimodal distribution was observed, albeit more prominent in the EV populations than the control. The line represents the mean.

FIG. 30—Comparison of Rab27DKO (Exosome-Deficient EVs) with normal EVs. All EV samples (normal and Rab27DKO) were significantly different than control for average total length of neurite (p<<<0.001). EVD1 and EVD2 were also significantly different than RabD1 and RabD1 (p<<0.01). There are no significant differences between EVD1 and EVD2 (p=0.56); similarly, no significant differences were noted between RabD1 and RabD2 (p=0.26).

FIG. 31—Effects of Electroporated Pcdh on Growth in OSNs. ‘P’ was used as a shorthanded abbreviation for Pcdh and P1_19 is the combined Pcdh1 and Pcdh19. There were significant differences in average neurite length compared to control for electroporated Pcdh19 and electroporated Pcdh1+19. No statistically significant differences, however, were observed for Pcdh1 compared to control (p=0.32).

FIG. 32A-32J—Expression pattern of Pcdh19 and other delta protocadherins in the epithelium. Members of the delta protocadherin family were assessed for expression at E17 (FIG. 32A-32H) and in adulthood (FIG. 32I, 32J). FIG. 32A) Pcdh1 is spatially differentially expressed in E17 epithelium. Black arrow indicates high levels of Pcdh1 expression in the dorsolateral regions of the epithelium. FIG. 32B) Pcdh19 is expressed in a noticeably different pattern from Pcdh1 in the epithelium. Black arrow shows less Pcdh19 in the dorsolateral epithelium as compared to Pcdh1. FIG. 32C-32G. Delta protocadherin family members are expressed in punctate patterns within the epithelium, consistent with expression in OSNs. Area shown corresponds to the region boxed in (FIG. 32A) for the various protocadherins. FIG. 32H) Olfactory marker protein (OMP) labels mature OSNs in the epithelium. FIG. 32I, FIG. 32J) Adult epithelial expression of Pcdh1 (FIG. 32I) and Pcdh19 (FIG. 32J). Black arrows point to dorsolateral epithelial regions analogous to that shown in (FIG. 32A, FIG. 32B). Less Pcdh19 is present in this region as compared to Pcdh1, similar to what was seen during development. Scale bar=25 um (FIG. 32C-32H) and 125 um (FIG. 32A-32B, FIG. 32I-32J).

FIG. 33A-33E—Generation of pcdh19E48X mutant mice. (FIG. 33A (SEQ ID NO: 1-2)) CRISPR was used to introduce a known stop mutation (pcdh19E48X) in humans into the corresponding position in mouse. A HindIII site was introduced downstream of the nonsense mutation for genotyping purposes. (FIG. 33B) Quantitative reverse transcriptase PCR was used to show reduced expression in both male hemizygous and female homozygous mice relative to controls, consistent with nonsense mediated decay. (FIG. 33C-33E) K562 cells (which normally do not form aggregates) were transfected with constructs bearing the engineered mutation and shown not to form aggregates. The pattern was similar to that seen in cells transfected with GFP alone (FIG. 33C). Co-transfection with a construct containing the intact Pcdh17 extracellular transmembrane domain showed cells were able to aggregate (FIG. 33E). Scale bar=50 um.

FIG. 34A-34F—pcdh19^{E48X} mice possess an increased number of lateral glomeruli. FIG. 34A) Control mice display the canonical medial (black arrowhead) and lateral (white arrowhead) glomeruli within the bulb. FIG. 34B) in mutants, there were more frequent examples of lateral glomeruli with two or sometimes three (white arrowheads) lateral projections. Applicant also found examples of medial glomeruli with multiple projections (black arrowheads). On occasion, examples of stray projections (black arrow) were also seen, although these smaller projections were not counted as additional glomeruli. FIG. 34C) male control mice had almost exclusively single glomeruli at the lateral position, with an occasional small secondary projection (white arrowheads). FIG. 34D) Male mutant showing two glomeruli on the lateral surface of approximately equal size (white arrowheads). FIG. 34E) Left lateral glomeruli were increased in number for both male and female mutants, with a trend towards significance in heterozygous mice. FIG. 34F) Right lateral glomeruli were only significantly affected in male mutants relative to control. The number of animals with three glomeruli were insufficient for statistical analysis but show an increase in mutants relative to control. n=12-14 animals per genotype and/or sex. Fisher's exact test with Bonferroni correction. Scale bar=500 um (FIG. 34A, 34B) or 200 um (FIG. 34C, 34D). *p<0.05%.

FIG. 35A-35K—pcdh19^{E48X} mice affect MOR28 OSN lateral coalescence. FIG. 35A-35H) Projection of image stack obtained by lightsheet microscopy (FIG. 35A, 35C, 35E, 35G) and segmented to identify projection patterns (FIG. 35B, 35D, 35F, 35H). FIG. 35B, 35D, 35H) Arrowhead indicate primary glomerular projection based on percent GFP expression relative to other projections (arrow). Asterisk indicates a projection that comprises less than 3% of the total GFP signal in the image stack. No primary projection could be easily determined in some samples (FIG. 35F). FIG. 35I) The number of lateral projections per hemibulb is increased in mutant and heterozygous animals; FIG. 35J) The percent of total GFP present in the main projection. FIG. 35K) Histogram of distribution of GFP expression within each projection (greater than 3%) in the various genotypes. Scale bar=100 um. *p<0.05, **p<0.01; ****p<0.0001. One way ANOVA with Bonferroni correction. n=5-8 animals per condition.

FIG. 36A-36K—pcdh19^{E48X} mice affect MOR28 OSN medial coalescence. FIG. 36A-36H) Images obtained by lightsheet microscopy (FIG. 36A, 36C, 36E, 36G) and segmented to identify projection patterns on the medial surface (FIG. 36B, 36D, 36F, 36H). Arrowheads indicate primary glomerular projection based on percent GFP expression relative to other projections (arrow). FIG. 36I) The number of medial projections per hemibulb is increased in mutant and heterozygous animals; FIG. 36J) The percent GFP present in the main medial projection. FIG. 36K) Histogram of distribution of GFP expression within each projection (greater than 3%) in the various genotypes. Scale bar=100 um. *p<0.05, **p<0.01. One way ANOVA with Bonferroni correction. n=4-8 animals per condition.

FIG. 37A-37K—pcdh19^{E48X} mice affect lateral MOR28 coalescence in males at 7 weeks. FIG. 37A-37H) Images obtained by lightsheet microscopy (FIG. 37A, 37C, 37E, 37G) and segmented to identify projection patterns (FIG. 37B, 37D, 37F, 37H). Arrowheads indicate primary glomerular projection based on percent GFP expression. FIG. 37I) The number of lateral projections per hemibulb. FIG. 37J) The percent GFP present in the main projection. FIG. 37K) Histogram of distribution of GFP expression within each projection (greater than 3%) in the various genotypes. Scale bar=100 um. *p<0.05. One way ANOVA with Bonferroni correction. n=3-7 animals per condition.

FIG. 38A-38C—MOR28 OSNs can be subdivided into two large clusters. FIG. 38A) Nanostring analysis followed by clustering of MOR28-GFP positive OSNs shows two clusters. FIG. 38B) individual genes (e.g., Pcdh17) can be expressed relatively highly in some cells (green) or weakly in others (purple). FIG. 38C) single cell qRT-PCR was used to confirm the Nanostring results.

FIG. 39A-39B—Male and female MOR28 OSNs can be distinguished based on expression. FIG. 39A) cluster analysis showing the most strongly differentially expressed genes between males and females. Note the variation in expression among different individual OSNs for any given gene. FIG. 39B) a subset of the same genes shown in (A) to indicate the difference in median expression between females and males.

FIG. 40A-40C—Pcdh19 promotes growth of DRG neurons in vitro. FIG. 40A. Fluorescent microscopic image of a DRG culture without protocadherin 19 (pcdh19). FIG. 40B. Fluorescent microscopic image of a DRG culture with protocadherin 19. FIG. 40C. Pcdh19 treatment increased neuron growth as expressed.

FIG. 41A-41E—Pcdh19 promotes longer neuron growth over bovine serum albumin (BSA) controls in vivo in transected sciatic nerves. FIG. 41A-41D shows fluorescent microscope images of growth after resection. FIG. 41E shows longer growth by measuring pixels of axons.

FIG. 42A-42B—EV treated DRG cultures grew more and longer neurites as compared to control treated DRG cultures.

FIG. 43A-43B—OSN derived EVs applied to transected sciatic nerves promoted improved axon organization during regeneration in vivo and may promote DRG regeneration.

FIG. 44—Schematic of technology for stamping EVs into patterns on coverslips or other suitable surfaces. After patterning, samples are dried on the surface.

FIG. 45—Microscopic image showing EVs and polyornithine pattern on a coverslip.

FIG. 46A-46B—Microscopic images demonstrating that OSN derived EVs patterned on a coverslip promote migration and extension of neurons along the pattern track.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g., given data set, art accepted standard, and/or with e.g., a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g., plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “antibody” refers to a protein or glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies.

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals.

As used herein, “aptamer” can refer to single-stranded DNA or RNA molecules that can bind to pre-selected targets including proteins with high affinity and specificity. Their specificity and characteristics are not directly determined by their primary sequence, but instead by their tertiary structure.

As used herein, “control” can refer to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, the terms “disease” or “disorder” are used interchangeably throughout this specification and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” can refer to physically discrete units suitable for use in a subject, each unit containing a predetermined quantity of the delta protocadherin composition described herein and/or a pharmaceutical formulation thereof calculated to produce the desired response or responses in association with its administration.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein. As used herein, the term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “gene” can refer to a hereditary unit corresponding to a sequence of DNA that occupies a specific location on a chromosome and that contains the genetic instruction for a characteristic(s) or trait(s) in an organism. The term gene also refers to translated and/or untranslated regions of a genome. “Gene” can refer to the specific sequence of DNA that is transcribed into an RNA transcript that can be translated into a polypeptide or be a catalytic or other type of RNA molecule, including but not limited to, tRNA, siRNA, piRNA, miRNA, long-non-coding RNA and shRNA.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides or polynucleotides of the present disclosure, unless stated otherwise.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

The term “modification causing said increased (or decreased) expression” refers to a modification in a gene which affects the expression level of that or another gene such that expression of that or another gene is increased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group of a mutation, a deletion and a frameshift. In particular embodiments, the modification is a mutation which results in increased or reduced expression of the functional gene product.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “modulate” or “modify” and variations of such terms broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “purified” or “purify” can be used in reference to a nucleic acid sequence, peptide, or polypeptide that has increased purity relative to the natural environment. A purified compound, compounds, molecules, or other substance can have enhanced, improved, and/or substantially different properties and/or effects as compared to the compound(s) and/or molecules in its natural state.

As used herein, the term “radiation sensitizer” refers to agents that can selectively enhance the cell killing from irradiation in a desired cell population, such as tumor cells, while exhibiting no single agent toxicity on tumor or normal cells.

As used interchangeably herein “decreased expression”, “reduced expression”, or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control. The term “modification causing said reduced expression” refers to a modification in a gene which affects the expression level of that or another gene such that the expression level of that or another gene is reduced or decreased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein, “separated” can refer to the state of being physically divided from the original source or population such that the separated compound, agent, particle, or molecule can no longer be considered part of the original source or population.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a disease, disorder, condition and/or symptom thereof further described elsewhere herein. The effect can be therapeutic in terms of a partial or complete cure of a disease, condition, symptom or adverse effect attributed to the disease, disorder, or condition. The term “treatment” as used herein covers any treatment of a disease, disorder, condition and/or symptom thereof further described elsewhere herein, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein in relation to polynucleotides and polypeptides, and more particularly to delta protocadherin genes and gene products, “diseased” refers to a mutant or modified variant of a wild-type or non-diseased variant that causes in whole or in part a disease, disorder, condition and/or a symptom thereof. In contrast, a non-diseased variant is a wild-type or variant thereof that does not cause, in whole or in part, a disease, disorder, condition and/or a symptom thereof.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### Overview

Spinal cord injuries, traumatic damage to peripheral neurons, or degeneration caused by neurodegenerative disorders can have significant lifelong implications. Further, impaired neuron growth and/or development, activity, and/or connectivity and/or synapse formation play roles in diseases, disorder, conditions and/or symptoms thereof, such as epilepsy and other seizure disorders, neurogenerative diseases, and/or the like. Understanding the mechanisms that can promote the repair of the nervous system has major clinical implications for human health.

Numerous environmental and genetic factors have been identified that can influence nervous system repair (Curcio M, Bradke F. Axon Regeneration in the Central Nervous System: Facing the Challenges from the Inside. Annu. Rev. Cell Dev. Biol. 2018 Oct. 6; 34:495-521 and Fawcett J W. The Struggle to Make CNS Axons Regenerate: Why Has It Been so Difficult? Neurochem Res. Springer US; 2020 Jan. 1; 45(1):144-58). Despite this, treatment options for repair are still highly limited. Current efforts span a variety of different approaches. These include using different cell-types to replace or encourage growth (e.g., stem cells, neural progenitors, or glial cells (Ahuja C S, Mothe A, Khazaei M, Badhiwala J H, Gilbert E A, van der Kooy D, et al. The leading edge: Emerging neuroprotective and neuroregenerative cell-based therapies for spinal cord injury. Stem Cells Transl Med. 2020 December; 9(12):1509-30. PMCID: PMC7695641), developing new conduits to promote regrowth (Carvalho C R, Oliveira J M, Reis R L. Modern Trends for Peripheral Nerve Repair and Regeneration: Beyond the Hollow Nerve Guidance Conduit. Front Bioeng Biotechnol. 2019; 7:337. PMCID: PMC6882937), identifying approaches to activate axon guidance pathways (Hilton B J, Bradke F. Can injured adult CNS axons regenerate by recapitulating development? Development. 2017 Oct. 1; 144(19):3417-29), treating individuals with drugs to reduce inflammation (Ren Y, Young W. Managing inflammation after spinal cord injury through manipulation of macrophage function. Neural Plast. 2013; 2013:945034. PMCID: PMC3833318), and adding purified proteins known to promote axonal sprouting and growth (Wu Q, Xiang Z, Ying Y, Huang Z, Tu Y, Chen M, et al. Nerve growth factor (NGF) with hypoxia response elements loaded by adeno-associated virus (AAV) combined with neural stem cells improve the spinal cord injury recovery. Cell Death Discov. 2021 Oct. 21; 7(1):301. PMCID: PMC8531363).

However, none of these treatments alone is typically successful. No one single protein, device, or treatment can overcome the many varied factors that can prevent proper regeneration. The Working Examples herein can at least demonstrate that delta protocadherins, particularly delta Protocadherin 19 (PCDH19) genes and gene products play a role in neural regeneration and development. PCDH19 is a member of the delta subfamily of protocadherins (Bisogni A J, Ghazanfar S, Williams E O, Marsh H M, Yang J Y, Lin D M. Tuning of delta-protocadherin adhesion through combinatorial diversity. Elife. eLife Sciences Publications Limited; 2018 Dec. 14; 7:400. PMCID: PMC6326727). All members of the subfamily are adhesion molecules, and several are associated with neurological disorders, including autism, depression, and bipolar disorders. Particularly noteworthy is the fact that mutations in PCDH19 are the causative mutation behind human PCDH19-girls clustering epilepsy (Depienne C, Leguern E. PCDH19-related infantile epileptic encephalopathy: an unusual X-linked inheritance disorder. Hum. Mutat. 2012 April; 33(4):627-34). The association with or causation of neurological disorders by members of this gene family underscores their importance in neuronal development.

With that said, embodiments disclosed herein provide compositions that include a delta protocadherin gene or gene product, a delta protocadherin modifier, or both. The compositions can be used to provide a functional protocadherin gene or gene product to a subject in need thereof and/or modify an endogenous delta protocadherin gene or gene product. In some embodiments, the delta protocadherin gene and/or gene product improves and/or enhances growth, regeneration, and/or development of a neuron. In some embodiments, the delta protocadherin gene and/or gene product improves and/or enhances neuron connectivity during growth, development, and/or regeneration. The modification can modify a diseased and/or dysfunctional protocadherin gene or gene product. In some embodiments, the modification can increased expression, function, and/or activity of an endogenous delta protocadherin gene or gene product so as to be at normal, substantially normal, or at a level that reduces or eliminates a disease, disorder, and/or condition or symptom thereof in a subject. In some embodiments, the modification can inhibit expression and/or activity of a mutated or diseased Pcdh 19 gene or gene product. The compositions and formulations thereof described herein can be used to treat or prevent a disease or disorder, such as a delta-protocadherin disease or disorder, particularly a Pcdh19 disease or disorder, a nerve palsy, injury, or other nerve damage, promote and/or enhance neuron growth, development, and/or regeneration, a neurodegenerative disease or disorder, and/or the like, and any combination thereof.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Delta Protocadherin Compositions

Described in several example embodiments herein are compositions that include a delta protocadherin gene or gene product, a delta protocadherin modifier, or both. The term “gene” is defined elsewhere herein. As the term is used herein “gene product” refers to any molecule produced from transcription of a gene and includes coding and non-coding products, including but not limited to, RNA molecules (including mRNA, tRNA, microRNA, long non-coding RNA, and/or the like), peptides, and polypeptides.

In some embodiments, the composition includes a delta protocadherin gene or gene product, a delta protocadherin modifier, or both. In certain example embodiments, the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof. In certain example embodiments, the delta protocadherin modifier is effective to modify a delta protocadherin gene or gene product, optionally where the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof.

In certain example embodiments, the delta protocadherin modifier is effective to increase or decrease expression and/or activity of the one or more delta protocadherin genes or gene products. In certain example embodiments, wherein the delta protocadherin modifier is effective to modify the gene or gene product polynucleotide and/or polypeptide sequence. In certain example embodiments, the delta protocadherin modifier is effective to cause insertions and/or deletions in the delta protocadherin gene.

In certain example embodiments, the delta protocadherin modifier comprises a genetic modification system (e.g., a CRISPR-Cas system, a zinc finger nuclease system, a meganuclease system), an RNA modification system (e.g., an RNAi system, an RNA-editing system (e.g., an ADAR or CRISPR-Cas-ADAR based system), a CRISPRi system) an antibody or fragment thereof, an aptamer, or any combination thereof.

In certain example embodiments, the delta protocadherin gene or gene product comprises a delta protocadherin encoding polynucleotide (e.g., DNA or RNA) or fragment thereof, a delta protocadherin polypeptide or functional fragment thereof, or any combination thereof.

In certain example embodiments, the composition comprises a delta protocadherin gene or gene product that is a functional delta protocadherin gene or gene product and a delta protocadherin modifier that inhibits a non-functional or aberrant delta protocadherin gene or gene product.

In certain example embodiments, the delta protocadherin gene or gene product, the delta protocadherin modifier, or both are contained in a vesicle, optionally an exosome or microvesicle. In certain example embodiments, the extracellular vesicles are olfactory derived extracellular vesicles, optionally olfactory sensory neuron derived extracellular vesicles. In certain example embodiments, the delta protocadherin gene or gene product, the delta protocadherin modifier, or both are native to the extracellular vesicle or exogenous to the extracellular vesicle.

In certain example embodiments, the composition further includes a cargo, wherein the cargo is optionally a polynucleotide, a polypeptide, a nutrient (e.g., lipid, amino acid, carbohydrate, peptide, protein, sugar, vitamin, mineral, and/or the like), genetic modifying system or component thereof, antibody or fragment thereof, aptamer, affibody, small molecule chemical agent (e.g., a therapeutic and/or prevention), an immunomodulator, a hormone, an antipyretic, an anxiolytic, an antipsychotic, an analgesic, an antispasmodic, an anti-inflammatory agent, an anti-epileptic, an anti-histamine, an anti-infective, a radiation sensitizer, a chemotherapeutic, or any combination thereof. Other cargos are described elsewhere herein.

In certain example embodiments, the composition comprises one or more targeting moieties coupled to and/or otherwise associated with the delta protocadherin gene or gene product, the delta protocadherin modifier, or both, wherein the targeting moiety is optionally a peptide, polypeptide, polynucleotide, sugar, a chemical molecule, a polymer, a lipid, a glycan, a peptidoglycan, or any combination or complex thereof (e.g., receptors, receptor ligands, antibodies and fragments thereof, aptamers, affibodies, antibody and/or aptamer epitopes, binding agents and their binding partners (e.g., biotin and streptavidin, enzymes and their substrates, a targeting nucleic acid, target nucleic acid and guided nuclease (e.g., miRNA, gRNA, RISC, Cas, etc.), guide nucleic acid for a guided nuclease system, and/or the like.

In certain example embodiments, the composition is frozen, dehydrated, lyophilized, or otherwise modified for storage.

The compositions can have one or more beneficial and/or therapeutic effects. In certain example embodiments, the composition is effective to stimulate axonal growth and/or increase the rate of axonal growth in a peripheral neuron, a central nervous system neuron, or both. In certain example embodiments, the composition the composition is effective to increase correct axonal connectivity during neuron regeneration.

### Delta Protocadherin Genes and Gene Products

### Polypeptides and Polynucleotides

In some embodiments, the composition includes one or more delta protocadherin polypeptides. “Delta protocadherin polynucleotides” includes full-length delta protocadherin polypeptides, functional variants thereof, and functional domains or fragments thereof. In some embodiments, the one or more delta protocadherin polypeptides included in the composition are selected from Pcdh1, Pcdh7, Pcdh8, Pcdh9, Pcdh10, Pcdh11, Pcdh17, Pcdh18, Pcdh19, Pcdh20 and any combination thereof.

In some embodiments, the composition includes one or more delta protocadherin polynucleotides. “Delta protocadherin polynucleotides” encode one or more delta protocadherin polypeptides previously described. In some embodiments, the delta protocadherin polynucleotides encode one or more delta protocadherin polypeptides selected from Pcdh1, Pcdh7, Pcdh8, Pcdh9, Pcdh10, Pcdh11, Pcdh17, Pcdh18, Pcdh19, Pcdh20 and any combination thereof. In some embodiments, the delta protocadherin polynucleotide(s) are codon optimized for expression in a eukaryotic cell, preferably a mammalian cell, more preferably a human cell. In some embodiments, the delta protocadherin polynucleotide(s) are codon optimized for expression in an olfactory cell, a neuron, a neuron glial cell, or an olfactory neuron or olfactory glial cell.

In some embodiments, the delta protocadherin polynucleotides can be included in a vector or vector system for production of one or more delta protocadherin polypeptides and/or delivery of said polynucleotides to a cell. Vectors and delivery are discussed in greater detail elsewhere herein.

### Delta Protocadherin Modifiers

Described in several example embodiments herein are agents capable of modifying a delta protocadherin gene or gene product (e.g., a delta protocadherin encoding polynucleotide and/or polypeptide). Such agents are also referred to as “delta protocadherin modifiers”. In some embodiments, delta protocadherin modifier is effective to modify a delta protocadherin gene or gene product, optionally where the delta protocadherin gene or gene product is Pcdh1, Pcdh7, Pcdh8, Pcdh9, Pcdh10, Pcdh11, Pcdh17, Pcdh18, Pcdh19, Pcdh20 or any combination thereof.

In some embodiments, the delta protocadherin modifier is effective to increase or decrease expression and/or activity of the delta protocadherin gene or gene product. In some embodiments, the expression of a non-diseased or normal delta protocadherin gene or gene product is increased, the expression of a diseased or mutant delta protocadherin gene or gene product is decreased, or both.

In some embodiments, the delta protocadherin modifier is effective to modify the polynucleotide and/or polypeptide sequence. In some embodiments, the modification is an insertion, deletion, substitution, or any combination thereof of one or more nucleotides of a delta protocadherin encoding polynucleotide. In some embodiments, the modification is an insertion, deletion, substitution, or any combination thereof of one or more amino acids of a delta protocadherin polypeptide.

In some embodiments the modification corrects (e.g., via insertion, deletion, and/or substitution) one or more of the mutations in a delta protocadherin gene, such as one or more mutations that are involved in the pathology of a disease, disorder, condition, and/or a symptom thereof.

In some embodiments, polynucleotide modification can include the introduction, deletion, or substitution of 1-75 nucleotides at one or more a random or target sequences of a polynucleotide and/or genome. The modification can include the introduction, deletion, and/or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at one or more a random or target sequences of a polynucleotide and/or genome. The modification can include the introduction, deletion, and/or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at one or more a random or target sequences of a polynucleotide and/or genome. The modification can include the introduction, deletion, and/or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at one or more a random or target sequences of a polynucleotide or genome. The modification can include the introduction, deletion, and/or substitution of 20, 21, 22, 23, 24, 25, 26, 27, 28, 29, 30, 35, 40, 45, 50, or 75 nucleotides at one or more a random or target sequences of a polynucleotide and/or genome. The modification can include the introduction, deletion, or substitution of 40, 45, 50, 75, 100, 200, 300, 400 or 500 nucleotides at one or more a random or target sequences of a polynucleotide and/or genome. The modification can include the introduction, deletion, and/or substitution of 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 at one or more a random or target sequences of a polynucleotide and/or genome. In some embodiments, the modification leads to the modified (e.g., increased or decreased expression of the delta protocadherin gene or gene product).

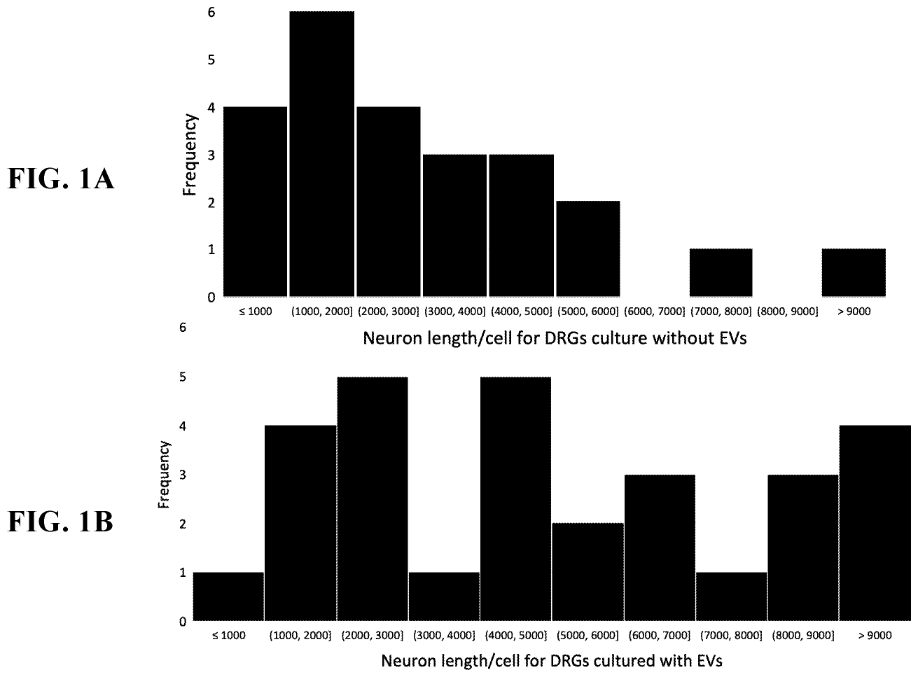

In some embodiments, the modification corrects (e.g., via insertion, deletion, and/or substitution) one or more of the mutations in a Pcdh19 gene and/or gene product. In some embodiments the one or more mutations in in a Pcdh19 gene and/or gene product that is/are corrected is/are a missense mutation (e.g., c.242T>G/p.Leu81Arg; c.437C>G/p.Thr146Arg; c.617T>A/p.Phe206Tyr; c.747A>T/p.Glu249Asp; c.1023C>G/p.Asp341Glu; c.1682C>G/p.Pro561Arg; c.1700C>T/p.Pro567Leu; C.1852G>A/p.Asp618Asn); a nonsense mutation (e.g., c.462C>A/p.Tyr154X; c.2656C>T/p.Arg886X); a base pair indel (e.g., c.424delG/p.Ala142ProfsX70; c.514dupG/p.Glu172GlyfsX54; c.697_700delinsTAAC/p.Asp233X; c.2019delC/p.Ser674LeufsX2); in-frame duplication (e.g., c.415_423dup/p.Ser139_Ala141dup); whole gene deletion (e.g., c695A>G (p.N232S) and c.1441G>T (pD481Y); those set forth in Kloc et al., particularly at FIG. 1 and Tables 1-2. See also Depienne et al. 2011. Hum Mutat. 2011 January; 32(1): E1959-E1975, particularly at Tables 1A-1B, 2; and Gursoy et al., Ann Indian Acad Neurol. 2020 March-April; 23(2): 206-210, particularly at Tables 1 and 2, Kloc et al., Molecular Psychiatry volume 24, pages 241-251 (2019), particularly at FIG. 1 and Tables 1-2.

In some embodiments, the modification is a replacement of a whole or part of a diseased delta protocadherin gene with a non-diseased delta protocadherin gene. In some embodiments, the modification is the insertion of a non-diseased delta protocadherin gene into the genome of a subject.

The delta protocadherin modifiers or components thereof can be provided as one or more polynucleotides, polypeptides, vectors, complexes thereof, or any combination thereof where suitable. In some embodiments, a delta protocadherin modifier polynucleotide is codon optimized for expression in a eukaryotic cell, preferably a mammalian cell, more preferably a human cell. In some embodiments, the delta protocadherin modifier polynucleotide(s) are codon optimized for expression in an olfactory cell, a neuron, a neuron glial cell, or an olfactory neuron or olfactory glial cell. In some embodiments, the delta protocadherin modifier polynucleotides can be included in a vector or vector system for production of one or more delta protocadherin polypeptides and/or delivery of said polynucleotides to a cell. Vectors and delivery are discussed in greater detail elsewhere herein.

### Genetic Modifying Agents

In certain embodiments, the one or more modulating agents may be a genetic modifying agent. The genetic modifying agent may comprise a programmable nuclease system (e.g., an RNA-guided system (e.g., a CRISPR (also referred to herein as a CRISPR-Cas system), a zinc finger nuclease system, a TALEN, a meganuclease), an RNAi system (e.g., antisense RNA, siRNA, and CRISPRi), RNA editors, or a combination thereof. In some embodiments, a delta protocadherin gene or gene product can be modified with the genetic modifying agents. Modifications are discussed elsewhere herein.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

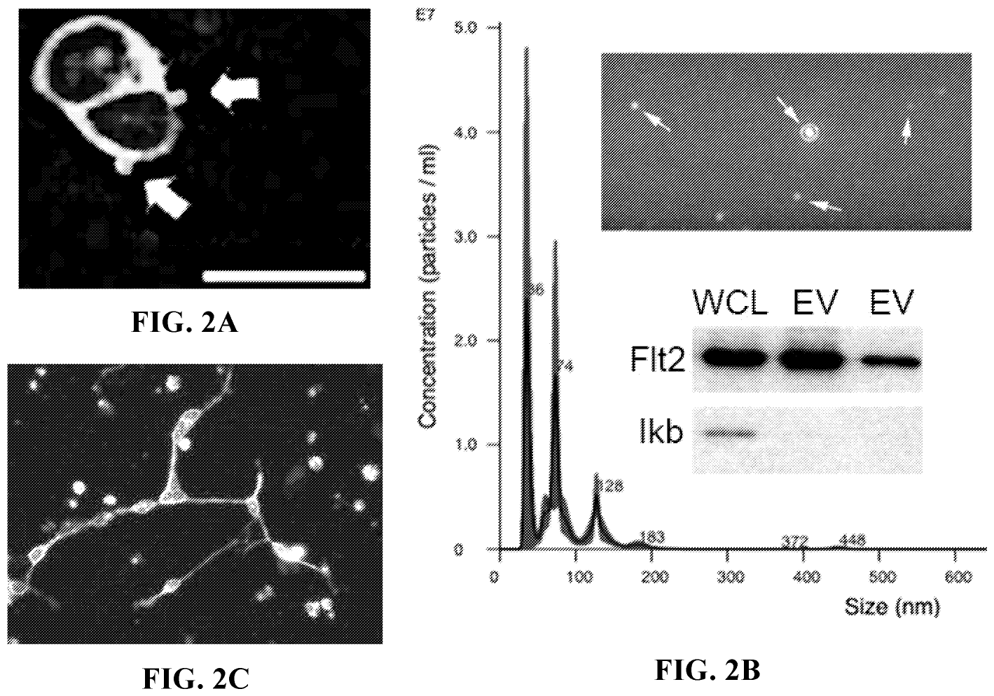

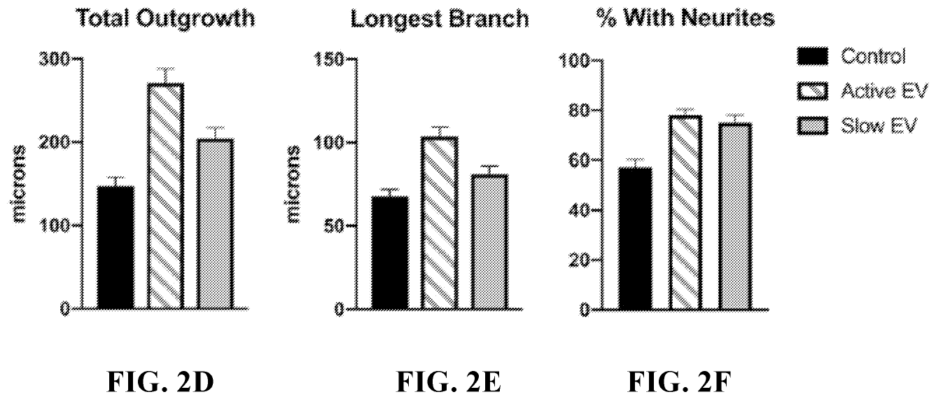

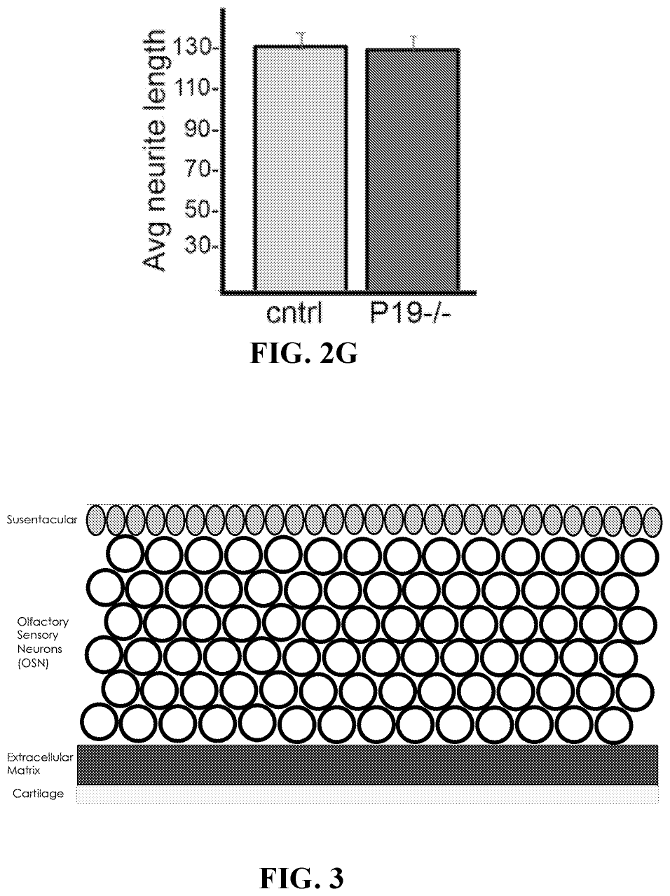

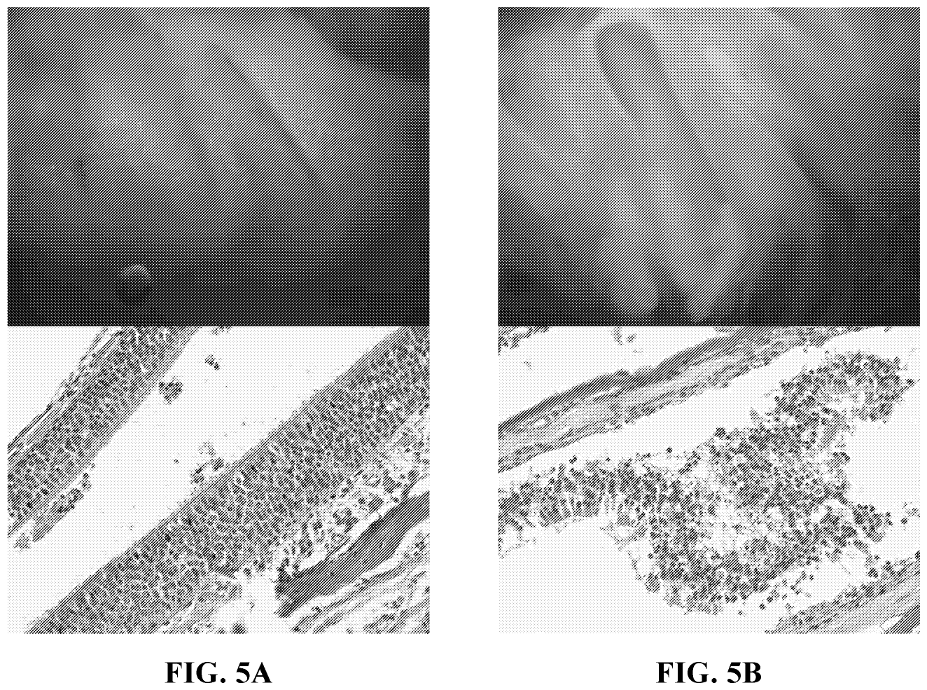

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9(1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 3) or PKKKRKVEAS (SEQ ID NO: 4); the NLS from nucleoplasmin (e.g., the nucleoplasmin bipartite NLS with the sequence KRPAATKKAGQAKKKK (SEQ ID NO: 5)); the c-myc NLS having the amino acid sequence PAAKRVKLD (SEQ ID NO: 6) or RQRRNELKRSP (SEQ ID NO: 7); the hRNPA1 M9 NLS having the sequence NQSSNFGPMKGGNFGGRSSGPYGGGGQYFAKPRNQGGY (SEQ ID NO: 8); the sequence RMRIZFKNKGKDTAELRRRRVEVSVELRKAKKDEQILKRRNV (SEQ ID NO: 9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO: 12) of human p53; the sequence SALIKKKKKMAP (SEQ ID NO: 13) of mouse c-abl IV; the sequences DRLRR (SEQ ID NO: 14) and PKQKKRK (SEQ ID NO: 15) of the influenza virus NS1; the sequence RKLKKKIKKL (SEQ ID NO: 16) of the Hepatitis virus delta antigen; the sequence REKKKFLKRR (SEQ ID NO: 17) of the mouse Mxl protein; the sequence KRKGDEVDGVDEVAKKKSKK (SEQ ID NO: 18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Donor Templates

In some embodiments, the CRISPR-Cas system includes a donor template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing Systems

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

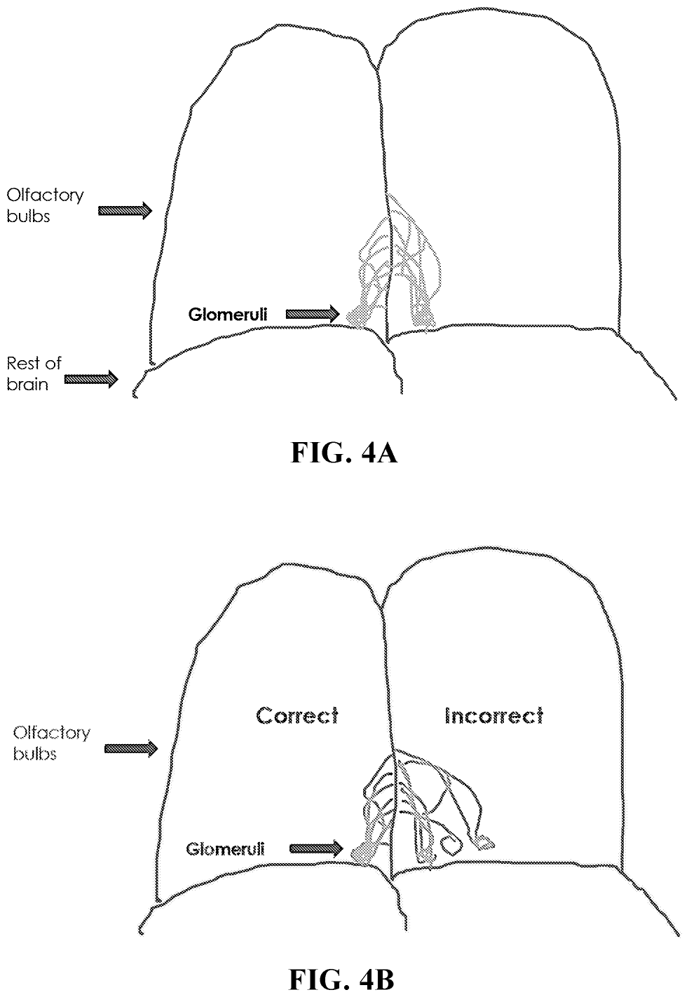

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125,126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 20)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 21)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is an RNA interference (RNAi) system (e.g., shRNA, antisense RNA, CRISPRi and/or the like). Such systems act to inhibit or in some configurations activate transcription and/or translation of a polynucleotide such that expression of the polynucleotide is reduced and/or functionally eliminated. Various mechanisms of action are employed by different systems to accomplish transcription and/or translation inhibition. As used herein, “gene silencing” or “gene silenced” in reference to an activity of an RNAi molecule or system where transcription and/or translation is inhibited or repressed such that expression of the gene is reduced, optionally to levels where no gene transcription or translation can be detected.

As used herein, the term “RNAi” refers to any type of interfering RNA or system that interferes with RNA transcription or translation, including but not limited to, siRNAi, shRNAi, endogenous microRNA and artificial microRNA, antisense RNA, CRISPRi, and/or the like. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

Any suitable RNAi molecule or system cans be used to modify the expression of a delta protocadherin gene. Such molecules and systems are generally known in the art and include without limitation, siRNA, shRNA, microRNA, piRNA, CRISPRi, antisense RNA, long non-coding RNA, and/or the like. See e.g., Setten et al., 2019, Nat Rev Drug Discov. 2019 June; 18(6):421-446. doi: 10.1038/s41573-019-0017-4; K. Lundstrom. Viruses. 2020 Aug. 23; 12(9):924. doi: 10.3390/v12090924; Saw et al., Sci China Life Sci. 2020 April; 63(4):485-500. doi: 10.1007/s11427-018-9438-y; Bajan et al. Cells. 2020 Jan. 7; 9(1):137. doi: 10.3390/cells9010137; Weng et al., Biotechnol Adv. 2019 September-October; 37(5):801-825. doi: 10.1016/j.biotechadv.2019.04.012; Dong et al., Adv Drug Deliv Rev. 2019 April; 144:133-147. doi: 10.1016/j.addr.2019.05.004; Hu et al., Signal Transduct Target Ther. 2020 Jun. 19; 5(1):101. doi: 10.1038/s41392-020-0207-x; Sajid et al., Pharmaceuticals (Basel). 2020 Oct. 7; 13(10):294. doi: 10.3390/ph13100294; Hsu et al., Biotechnol Adv. 2019 December; 37(8):107447. doi: 10.1016/j.biotechadv.2019.107447; Peddle et al., Int J Mol Sci. 2020 Mar. 27; 21(7):2329. doi: 10.3390/ijms21072329; Adiego-Perez et al., FEMS Microbiol Lett. 2019 Apr. 1; 366(8):fnz086. doi: 10.1093/femsle/fnz086; Anton et al., Biol Methods Protoc. 2018 May 10; 3(1):bpy002. doi: 10.1093/biomethods/bpy002; Nishida and Kondo. Metab Eng. 2021 January; 63:141-147. doi: 10.1016/j.ymben.2020.12.002; Kondrateva et al., Gene. 2021 Feb. 15; 769:145225.doi: 10.1016/j.gene.2020.145225, which are incorporated by reference herein and can be adapted for use with the present embodiments.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Delivery

The present disclosure also provides delivery systems for introducing components of the delta protocadherin compositions described elsewhere herein to cells, tissues, organs, or organisms. A delivery system may comprise one or more delivery vehicles and/or cargos. Exemplary delivery systems and methods include, but are not limited to, those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

### Physical Delivery

In some embodiments, the delta protocadherin compositions may be introduced to cells by physical delivery methods. Examples of physical methods include microinjection, electroporation, and hydrodynamic delivery. Both nucleic acid and proteins may be delivered using such methods. For example, a delta protocadherin protein may be prepared in vitro, isolated, (refolded, purified if needed), and introduced to cells.

### Microinjection

Microinjection of the delta protocadherin compositions directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for delta protocadherin proteins, genetic modifying system proteins (e.g., a Cas) and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus and modification of e.g., a delta protocadherin gene.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the delta protocadherin compositions and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the delta protocadherin compositions, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transection

The delta protocadherin compositions, e.g., nucleic acids and/or polypeptides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The delta protocadherin compositions, e.g., nucleic acids and/or polypeptides, can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer be used to transduce cells.

### Biolistics

The delta protocadherin compositions, e.g., nucleic acids and/or polypeptides, can be introduced to cells using a biolistic method or technique. The term of art “biolistic”, as used herein refers to the delivery of nucleic acids to cells by high-speed particle bombardment. In some embodiments, the cargo(s) can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a delta protocadherin compositions or component thereof described herein. Various implantable devices are described in the art, and include any device, graft, or other composition that can be implanted into a subject.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles in accordance with the present invention may a greatest dimension (e.g., diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to plant cells, e.g., as described in WO 2008042156, US 20130185823, and WO2015089419. In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured, and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, describing particles, methods of making and using them and measurements thereof.

### Vectors

Also provided herein are vectors that can contain one or more of the delta protocadherin compositions described herein. In aspects, the vector can contain one or more polynucleotides encoding one or more elements of delta protocadherin compositions described herein. The vectors can be useful in producing bacterial, fungal, yeast, plant cells, animal cells, and transgenic animals that can express one or more of the delta protocadherin compositions described herein. Within the scope of this disclosure are vectors containing one or more of the polynucleotide sequences described herein. One or more of the polynucleotides that are part of the delta protocadherin compositions described herein can be included in a vector or vector system. The vectors and/or vector systems can be used, for example, to express one or more of the polynucleotides in a cell, such as a producer cell, to produce delta protocadherin compositions described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

In some a embodiments, the vector can be a bicistronic vector. In some embodiments, a bicistronic vector can be used for one or more delta protocadherin compositions described herein. In some embodiments, expression of the delta protocadherin compositions described herein can be driven by the CBh promoter or other ubiquitous promoter. Where the element of the delta protocadherin composition is an RNA, its expression can be driven by a Pol III promoter, such as a U6 promoter. In some embodiments, the two are combined.

### Cell-Based Vector Amplification and Expression

Vectors can be designed for expression of one or more delta protocadherin compositions described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include but are not limited to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of the delta protocadherin compositions so as to drive expression of the one or more elements of the delta protocadherin compositions described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of the delta protocadherin compositions described herein are introduced into a host cell such that expression of the elements of the engineered delivery system described herein direct formation of the delta protocadherin compositions (including, but not limited to, a delta protocadherin modifier, which is described in greater detail elsewhere herein). For example, different elements of the delta protocadherin compositions described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the delta protocadherin compositions described herein that incorporates one or more elements of the delta protocadherin compositions described herein or contains one or more cells that incorporates and/or expresses one or more elements of the delta protocadherin compositions described herein.

In some embodiments, two or more of the elements expressed from the same or different regulatory element(s), can be combined in a single vector, with one or more additional vectors providing any components of the system not included in the first vector. Delta protocadherin polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more delta protocadherin proteins, embedded within one or more intron sequences (e.g., each in a different intron, two or more in at least one intron, or all in a single intron). In some embodiments, the delta protocadherin polynucleotides can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In aspects, the polynucleotides and/or vectors thereof described herein can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Fer1l4), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include, but is not limited to, sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more elements of the delta protocadherin compositions described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., aspects of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing a delta protocadherin polynucleotide to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the delta protocadherin polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the delta protocadherin polynucleotide such that the selectable marker polypeptide, when translated, is inserted between two amino acids between the N- and C-terminus of the delta protocadherin polypeptide or at the N- and/or C-terminus of the delta protocadherin polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the delta protocadherin compositions described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the delta protocadherin compositions described herein via suitable linker, such as a glycine or glycine serine linkers. Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some a embodiments, the targeting moiety encoding polynucleotides can be included in the vector or vector system such that the delta protocadherin polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated delta protocadherin (s) to specific cells, tissues, organs, etc.

### Cell-Free Vector and Polynucleotide Expression

In some aspects, the polynucleotide encoding one or more features of the delta protocadherin can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some aspects, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Non-Viral Vectors and Carriers

In some aspects, the vector is a non-viral vector or carrier. In some aspects, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with a delta protocadherin polynucleotide of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid, polynucleotide molecule, or composition that be attached to or otherwise interact with, encapsulate, and/or associate with a polynucleotide to be delivered, such as a delta protocadherin polynucleotide of the present invention.

### Naked Polynucleotides

In some aspects, one or more delta protocadherin polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the delta protocadherin polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g. plasmids and artificial chromosomes), molecules that contain portions that are single stranded and portions that are double stranded (e.g., ribozymes), and the like. In some aspects, the naked polynucleotide contains only the delta protocadherin polynucleotide(s) of the present invention. In some aspects, the naked polynucleotide can contain other nucleic acids and/or polynucleotides in addition to the delta protocadherin polynucleotide(s) of the present invention. The naked polynucleotides can include one or more elements of a transposon system. Transposons and system thereof are described in greater detail elsewhere herein.

### Non-Viral Polynucleotide Vectors

In some aspects, one or more of the delta protocadherin polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some aspects, the non-viral polynucleotide vector can have a conditional origin of replication. In some aspects, the non-viral polynucleotide vector can be an ORT plasmid. In some aspects, the non-viral polynucleotide vector can have a minimalistic immunologically defined gene expression. In some aspects, the non-viral polynucleotide vector can have one or more post-segregationally killing system genes. In some aspects, the non-viral polynucleotide vector is AR-free. In some aspects, the non-viral polynucleotide vector is a minivector. In some aspects, the non-viral polynucleotide vector includes a nuclear localization signal. In some aspects, the non-viral polynucleotide vector can include one or more CpG motifs. In some aspects, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In aspects, the S/MAR sequence is located downstream of an actively transcribed polynucleotide (e.g., one or more delta protocadherin polynucleotides of the present invention) included in the non-viral polynucleotide vector. In some aspects, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some aspects, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some aspects, the non-viral polynucleotide vector can be a retrotransposon vector. In some aspects, the retrotransposon vector includes long terminal repeats. In some aspects, the retrotransposon vector does not include long terminal repeats. In some aspects, the non-viral polynucleotide vector can be a DNA transposon vector. DNA transposon vectors can include a polynucleotide sequence encoding a transposase. In some aspects, the transposon vector is configured as a non-autonomous transposon vector, meaning that the transposition does not occur spontaneously on its own. In some of these aspects, the transposon vector lacks one or more polynucleotide sequences encoding proteins required for transposition. In some aspects, the non-autonomous transposon vectors lack one or more Ac elements.

In some aspects, a non-viral polynucleotide transposon vector system can include a first polynucleotide vector that contains the delta protocadherin polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the delta protocadherin polynucleotide(s) of the present invention) and integrate it into one or more positions in the host cell's genome. In some aspects the transposon vector or system thereof can be configured as a gene trap. In some aspects, the TTRs can be configured to flank a strong splice acceptor site followed by a reporter and/or other gene (e.g., one or more of the delta protocadherin polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Chemical Carriers

In some aspects, the delta protocadherin polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the delta protocadherin polynucleotide(s) of the present invention), (2) those capable of targeting specific cells, (3) those capable of increasing delivery of the polynucleotide (such as the delta protocadherin polynucleotide(s) of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the delta protocadherin components described herein. Suitable sizes include macro-, micro-, and nano-sized particles.

In some aspects, the non-viral carrier can be an inorganic particle. In some aspects, the inorganic particle, can be a nanoparticle. The inorganic particles can be configured and optimized by varying size, shape, and/or porosity. In some aspects, the inorganic particles are optimized to escape from the reticulo endothelial system. In some aspects, the inorganic particles can be optimized to protect an entrapped molecule from degredation, the Suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, poarticles, and materials, (e.g., supermagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some aspects, the non-viral carrier can be lipid-based. Suitable lipid-based carriers are also described in greater detail herein. In some aspects, the lipid-based carrier includes a cationic lipid or an amphiphilic lipid that is capable of binding or otherwise interacting with a negative charge on the polynucleotide to be delivered (e.g., such as an delta protocadherin polynucleotide of the present invention). In some aspects, chemical non-viral carrier systems can include a polynucleotide such as the delta protocadherin polynucleotide(s) of the present invention) and a lipid (such as a cationic lipid). These are also referred to in the art as lipoplexes. Other aspects of lipoplexes are described elsewhere herein. In some aspects, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g. the delta protocadherin polynucleotide(s) of the present invention). In some aspects, the lipid-based non-viral carrier can be a solid lipid particle or nanoparticle.

In some aspects, the non-viral carrier can be peptide-based. In some aspects, the peptide-based non-viral carrier can include one or more cationic amino acids. In some aspects, 35 to 40, 45, 50, 55, 60, 65, 70, 75, 80, 85, 90, 95, 99 or 100% of the amino acids are cationic. In some aspects, peptide carriers can be used in conjunction with other types of carriers (e.g., polymer-based carriers and lipid-based carriers to functionalize these carriers). In some aspects, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the delta protocadherin polynucleotides of the present invention), polymethacrylate, and combinations thereof.

In some aspects, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some aspects, the non-viral carrier can be a particle that is configured includes one or more of the delta protocadherin polynucleotides describe herein and an environmental triggering agent response element, and optionally a triggering agent. In some aspects, the particle can include a polymer that can be selected from the group of polymethacrylates and polyacrylates. In some aspects, the non-viral particle can include one or more aspects of the compositions microparticles described in US Pat. Pubs. 20150232883 and 20050123596, whose techniques and compositions can be adapted for use in the present invention.

In some aspects, the non-viral carrier can be a polymer-based carrier. In some aspects, the polymer is cationic or is predominantly cationic such that it can interact in a charge-dependent manner with the negatively charged polynucleotide to be delivered (such as the delta protocadherin polynucleotide(s) of the present invention). Polymer-based systems are described in greater detail elsewhere herein.

### Viral Vectors

In some aspects, the vector is a viral vector. The term of art “viral vector” and as used herein in this context refers to polynucleotide based vectors that contain one or more elements from or based upon one or more elements of a virus that can be capable of expressing and packaging a polynucleotide, such as a delta protocadherin polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the delta protocadherin compositions described herein. The viral vector can be part of a viral vector system involving multiple vectors. In some aspects, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other aspects of viral vectors and viral particles produce therefrom are described elsewhere herein. In some aspects, the viral vectors are configured to produce replication incompetent viral particles for improved safety of these systems.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the delta protocadherin compositions can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-HLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some aspects, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the delta protocadherin compositions described herein.

In some aspects, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some aspects, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some aspects, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some aspects, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some aspects, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some aspects, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In aspects, the third-generation lentiviral vector system can include at least two packaging vectors, with the gag-pol being present on a different vector than the rev gene.

In some aspects, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the delta protocadherin compositions.

In some aspects, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some aspects, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some aspects, the tropism of the resulting lentiviral particle can be tuned by incorporating cell targeting peptides into a lentiviral vector such that the cell targeting peptides are expressed on the surface of the resulting lentiviral particle. In some aspects, a lentiviral vector can contain an envelope protein that is fused to a cell targeting protein (see e.g. Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some aspects, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these aspects, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some aspects, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some aspects, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some aspects, the PDZ1 protein can be fused to an envelope protein, which can optionally be binding deficient and/or fusion competent virus envelope protein and included in a lentiviral vector. In some aspects, the TEFCA can be fused to a cell targeting peptide and the TEFCA-CPT fusion construct can be incorporated into the same or a different lentiviral vector as the PDZ1-envelope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver a delta protocadherin polynucleotide described herein to a cell.

In some aspects, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g. antibiotic resistance genes), Psi (4), RRE (rev response element), cPPT (central polypurine tract), promoters, WPRE (woodchuck hepatitis posttranscriptional regulatory element), SV40 polyadenylation signal, pUC origin, SV40 origin, F1 origin, and combinations thereof.

### Adenoviral Vectors, Helper-Dependent Adenoviral Vectors, and Hybrid Adenoviral Vectors

In some aspects, the vector can be an adenoviral vector. In some aspects, the adenoviral vector can include elements such that the virus particle produced using the vector or system thereof can be serotype 2 or serotype 5. In some aspects, the polynucleotide to be delivered via the adenoviral particle can be up to about 8 kb. Thus, in some aspects, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some aspects, the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In aspects of the helper-dependent adenoviral vector system one vector (the helper) can contain all the viral genes required for replication but contains a conditional gene defect in the packaging domain. The second vector of the system can contain only the ends of the viral genome, one or more delta protocadherin polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the delta protocadherin polynucleotides described herein. In some aspects, the polynucleotide to be delivered via the viral particle produced from a helper-dependent adenoviral vector or system thereof can be up to about 37 kb. Thus, in some aspects, a adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some aspects, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some aspects, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the delta protocadherin compositions. In some aspects, a hybrid-adenoviral vector can include one or more features of a retrovirus and/or an adeno-associated virus. In some aspects, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the delta protocadherin compositions described herein. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the delta protocadherin compositions described herein.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments, the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008) at Table 3.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the delta protocadherin polynucleotide(s)).

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the delta protocadherin compositions of the present invention. In some embodiments, where an HSV vector or system thereof is utilized, the host cell can be a complementing cell. In some embodiments, HSV vector or system thereof can be capable of producing virus particles capable of delivering a polynucleotide cargo of up to 150 kb. Thus, in some aspect, the delta protcadherein polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the delta protocadherin of the present invention.

### Poxvirus Vectors

In some embodiments, the vector can be a poxvirus vector or system thereof. In some embodiments, the poxvirus vector can result in cytoplasmic expression of one or more e delta protocadherin polynucleotides of the present invention. In some embodiments, the capacity of a poxvirus vector or system thereof can be about 25 kb or more. In some embodiments, a poxivirus vector or system thereof can include a one or more delta protocadherin polynucleotides of the present invention.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a delta protocadherin composition described herein are as used in the foregoing documents, such as WO 2014/093622 (PCT/US2013/074667) and are discussed in greater detail herein.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g. pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a delta protocadherin polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g. the delta protocadherin polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the delta protocadherin polynucleotide(s)) between 2 ITRs; (2) a vector that carries the AAV Rep-Cap encoding polynucleotides; and (3) helper polynucleotides. One of skill in the art will appreciate various methods and variations thereof that are both helper and -helper free and as well as the different advantages of each system.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., delta protocadherin transcripts, proteins, enzymes, mutant forms thereof, fusion proteins thereof, etc.), and virus particles (such as from viral vectors and systems thereof).

One or more delta protocadherin polynucleotides can be delivered using adeno associated virus (AAV), lentivirus, adenovirus or other plasmid or viral vector types, in particular, using formulations and doses from, for example, US Patents Nos. U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some aspects, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of delta protocadherin composition components (e.g., polynucleotides encoding delta protocadherin polypeptides) to cells via particles. In some embodiments, any of the of the delta protocadherin composition components (e.g., polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in aspects, particle delivery can also be advantageous for other delta protocadherin molecules and formulations described elsewhere herein.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of a delta protocadherin polypeptide) and/or RNA molecules (e.g., mRNA of a delta protocadherin polynucleotide). In certain cases, LNPs may be use for delivering RNP complexes of delta protocadherin polynucleotides/delta protocadherin polypeptides.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, an LNP delivery vehicle can be used to deliver a virus particle containing a delta protocadherin composition and/or component(s) thereof. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1: 1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037. [Epub ahead of print]; Altmoǧlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy—Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316;

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing a delta protocadherin composition and/or component(s) thereof. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g. http://cshprotocols.cshlp.org/content/2010/4/pdb.prot5407.long, the teachings of which can be applied and/or adapted to generated and/or deliver the CRISPR-Cas systems described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the CRISPR-Cas systems described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos, e.g., a delta protocadherin composition RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29: 341; [1401] El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Olfactory EVs

In some embodiments the delivery vehicle is an olfactory cell derived extracellular vesicle (e.g., an exosome and/or macrovesicle). In some embodiments the delivery vehicle is an olfactory neuron derived extracellular vesicle. In some embodiments, the delivery vehicle is an olfactory ensheathing or other olfactory glial cell derived EV. Such EVs are described in greater detail elsewhere herein.

### Devices

In some embodiments, EVs, such as those that can be used to deliver a protocadherin, or protocadherins can be stamped and patterned on the surface of a device. The device can be implanted in a subject, such as where nerve regrowth is desired. In some embodiments, the EVs are dried on the surface of the implant in the desired pattern. In some embodiments, the EVs are patterned along with one or more polymers, such as a biocompatible polymer. In some embodiments, the polymer is a polyornithine. In some embodiments, the patterns on the device can be designed such that they form correct or desired nerve tracts. Without being bound by theory, when the device is implanted into a subject the EVs and/or protocadherin(s), optionally protocadherin 19, present in the EV or otherwise on the device can stimulate nerve (e.g., axon) growth along the pattern.

### Targeted Delivery

In some embodiments, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference and the teachings of which can be applied and/or adapted for targeted delivery of one or more delta protocadherin compositions described herein. Mention is also made of International Patent Publication No. WO 2016/027264, and the documents it cites, all of which are incorporated herein by reference, the teachings of which can be applied and/or adapted for targeted delivery of one or more delta protocadherin compositions described herein. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference, the teachings of which can be applied and/or adapted for targeted delivery of one or more delta protocadherin compositions described herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR is higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein discussed, there is an embodiment of the invention wherein the delivery system comprises such a targeting or active targeting moiety. Likewise, the Table TBD below provides exemplary targeting moieties that can be used in the practice of the invention an as to each an embodiment of the invention provides a delivery system that comprises such a targeting moiety. Other suitable targeting moieties are described elsewhere herein.

TABLE
TBD - Exemplary targeting moieties

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine- | avβ3 | glioblastoma cells, human |
| aspartic acid-tyrosine- | | umbilical vein endothelial cells, |
| cysteine peptide | | tumor angiogenesis |
| (c(RGDyC)-LP) | | |
| ASSHN peptide | | endothelial progenitor cells; anti- |
| | | cancer |
| PR_b peptide | α₅β₁ integrin | cancer cells |
| AG86 peptide | α₆β₄ integrin | cancer cells |
| KCCYSL (P6.1 | HER-2 receptor | cancer cells |
| peptide) | | |
| affinity peptide LN | Aminopeptidase N | APN-positive tumor |
| (YEVGHRC) | (APN/CD13) | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more delta protocadherin compositions described herein.

Other exemplary targeting moieties are described elsewhere herein, such as epitope tags and the like.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g. chemical composition, etc.), and a biologic or physiologic stimuli (e.g., environmental pH, osmolarity, salinity, biologic molecule, etc.). In some embodiments, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extracellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. An MMP2-cleavable octapeptide (Gly-Pro-Leu-Gly-Ile-Ala-Gly-Gln (SEQ ID NO: 22)) can be incorporated into a linker, and can have antibody targeting, e.g., antibody 2C5.

The invention also comprehends light-or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe₃O₄ or γ-Fe₂O₃, e.g., those that are less than 10 nm in size. Targeted delivery can be then by exposure to a magnetic field.

### Modified Cells and Organisms

Described herein are modified cells that have been modified to express one or more one or more of the delta protocadherin compositions described in greater detail elsewhere herein. The cells may be further modified to produce a cargo molecule which can then be incorporated into a delivery vehicle produced by the modified cell. The cells that are modified can be any suitable mammalian cell. In some embodiments, the cells that are modified are olfactory cells. In some embodiments the cells that are modified are neurons. In some embodiments, the cells that are modified are neuroglial or glial cells (e.g., astrocytes, microglia, oligodendrocytes, radial glial cells, Schwan cells, ensheathing cells, and/or the like), astrocytes. In some embodiments, the olfactory cells that are modified are olfactory neurons or olfactory glial cells. The cells can be modified using any suitable genetic modification technique and or system, including but not limited to those described elsewhere herein. Others will be appreciated by those of ordinary skill in the art in view of the description herein.

The modified cells can be produced by a modified organism. Thus, also described herein are modified organisms, such as non-human mammalian species (including but not limited to non-human primates) that are modified to contain one or more cells, particularly one or more neurons, and more particularly one or more olfactory neuron cells, that express or over express one or more of the delta protocadherin molecules described in greater detail elsewhere herein.

In some embodiments, the modified cells can be included in a pharmaceutical formulation described elsewhere herein and/or administered to a subject in need thereof where they can produce the delta protocadherin compositions described elsewhere herein.

In some embodiments, the cells and/or organisms can be used to produce one or more of the delta protocadherin compositions described herein and/or delivery vehicle that may be suitable for administration to a subject in need thereof. In some embodiments, the produced delta protocadherin compositions can be isolated and/or purified and included in a formulation and/or delivered to a subject in need thereof.

### Formulations

### Pharmaceutical Formulations

Also described herein are pharmaceutical formulations that can contain an amount, effective amount, and/or least effective amount, and/or therapeutically effective amount of one or more compositions (which are also referred to as the primary active agent or ingredient elsewhere herein), cells, vectors, particles, and/or the like described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a delta protocadherin gene or gene product, a delta protocadherin modifier, or both as described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, the compositions described in greater detail elsewhere herein can be provided to a subject in need thereof as an ingredient, such as an active ingredient or agent, in a pharmaceutical formulation. Where appropriate, a compound or composition contained in the in the formulation can be formulated as a pharmaceutically acceptable salt thereof. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has, has had, and/or is suspected of having a nerve injury, nerve death, aberrant neuron connectivity, aberrant neuron activity, a neuropathy, or any combination thereof. In some embodiments, the subject in need thereof has, has had, and/or is suspected of having a neurodegenerative disease, disorder, and/or condition. In some embodiments, the subject in need thereof has, has had, and/or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, and/or is suspected of having a CNS neuron/nerve and/or a peripheral neuron/nerve injury, disease, disorder, and/or condition.

In some embodiments, the subject in need thereof has, has had, and/or is suspected of having an epilepsy, a seizure disease, disorder or condition, or a disease, disorder, or condition in which seizures are a symptom or result of the disease, disorder, or condition, including but not limited to non-epileptic seizures. In some embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In some embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, or is suspected of having a CNS neuron/nerve and/or a peripheral neuron/nerve injury, disease, disorder, and/or condition. In some embodiments, the disease, disorder, and/or condition is a genetic disease, disorder, and/or condition. In some embodiments, the disease, disorder, and/or condition is not a genetic disease, disorder, and/or condition.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules (including, but not limited to, e.g. polynucleotides, amino acids, peptides, polypeptides, antibodies and fragments thereof, aptamers, ribozymes, hormones, and/or the like), affibodies, immunomodulators, antipyretics, anxiolytics, antipsychotics, analgesics, antispasmodics, anti-inflammatories, anti-histamines, anti-infectives, an anti-epileptic agent, neurotransmitter agonists, neurotransmitter antagonists, chemotherapeutics, a nutrient (e.g., lipid, amino acid, carbohydrate, peptide, protein, sugar, vitamin, mineral, and/or the like), a small molecule chemical agent (e.g., a therapeutic or prevention), genetic modifying system or component thereof (e.g., a CRISPR-Cas system, Zinc Finger Nuclease system, a Mega nuclease system, an ADAR, a base editing system, RNAi or other gene silencing system, and/or the like), and any combination thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are increased neuron growth and/or regeneration, increased axon length, growth, and/or regeneration, and/or increased rate of neuron and/or axon growth and/or regeneration. In some embodiments, the one or more therapeutic effects are increased correct axon connectivity. This refers to the axon connecting to the appropriate target neuron or neurons during regeneration so as to be more similar to and/or like a pre-disease or pre-injured/damaged state. In some embodiments, the one or more therapeutic effects is or includes improved neuron and/or axon structure and organization during regeneration. This refers to the overall alignment, spacing, and/or positioning of the regenerating neurons so as to be more similar to and/or like a pre-disease or pre-injured/damaged state.

The effective amount, least effective amount, and/or therapeutically effective amount of the primary and optional secondary active agent described elsewhere herein contained in the pharmaceutical formulation, when present, can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration of the primary and optional secondary active agent described elsewhere herein contained in the pharmaceutical formulation, when present, can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent described elsewhere herein contained in the pharmaceutical formulation, when present, can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent when present in the pharmaceutical formulation can be present at any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where an EV or cell population is present in the pharmaceutical formulation (e.g., as a primary and/or or secondary active agent), the effective amount of EVs or cells is 1 or 2 cells or EVs to 1×10¹ cells or EVs/mL, 1×10²⁰ cells or EVs/mL or more, such as about 1×10¹ cells or EVs/mL, 1×10² cells or EVs/mL, 1×10³ cells or EVs/mL, 1×10⁴ cells or EVs/mL, 1×10⁵ cells or EVs/mL, 1×10⁶ cells or EVs/mL, 1×10⁷ cells or EVs/mL, 1×10⁸ cells or EVs/mL, 1×10⁹ cells or EVs/mL, 1×10¹⁰ cells or EVs/mL, 1×10¹¹ cells or EVs/mL, 1×10¹² cells or EVs/mL, 1×10¹³ cells or EVs/mL, 1×10¹⁴ cells or EVs/mL, 1×10¹⁵ cells or EVs/mL, 1×10¹⁶ cells or EVs/mL, 1×10¹⁷ cells or EVs/mL, 1×10¹⁸ cells or EVs/mL, 1×10¹⁹ cells or EVs/mL, t about 1×10²⁰/or EVs cells/mL, 1×10³⁰ cells or EVs/mL, 1×10⁴⁰ cells or EVs/mL, 1×10⁵⁰ cells or EVs/mL, 1×10⁷⁰ cells or EVs/mL, 1×10⁶⁰ cells or EVs/mL, 1×10⁸⁰ cells or EVs/mL, 1×10⁹⁰ cells or EVs/mL, or/to about 1×10¹⁰⁰ cells or EVs/mL or more.

In some embodiments, the amount or effective amount, particularly where an infective particle is being delivered (e.g., a virus particle carrying an active agent), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome or EV. In these embodiments, primary active ingredient(s), and/or optional secondary active ingredient(s), and/or pharmaceutically acceptable salt thereof where appropriate are incorporated into a liposome or EV. In embodiments where the dosage form is a liposome or EV, the pharmaceutical formulation is thus a liposomal or EV formulation. The liposomal or EV formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulation. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

In some embodiments, the dosage form is adapted for targeted delivery to a peripheral nerve, such as using any of the approaches discussed in e.g., Langert and Brey (Front. Neurosci. 2018 https://doi.org/10.3389/fnins.2018.00887), which is incorporated by reference herein.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, a polynucleotide, a polypeptide, a nutrient (e.g., lipid, amino acid, carbohydrate, peptide, protein, sugar, vitamin, mineral, and/or the like), genetic modifying system or component thereof, antibody or fragment thereof, aptamer, ribozymes, affibody, small molecule chemical agent (e.g., a therapeutic and/or prevention), an immunomodulator, a hormone, an antipyretic, an anxiolytic, an antipsychotic, an analgesic, an antispasmodic, an anti-inflammatory agent, an anti-epileptic agent, an anti-histamine, an anti-infective, a growth factor, a radiation sensitizer, a chemotherapeutic, a neurotransmitter agonist, a neurotransmitter antagonist, or any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Devices

In some embodiments, one or more delta protocadherins (e.g., Pcdh1, Pcdh7, Pcdh8, Pcdh9, Pcdh10, Pcdh 11, Pcdh17, Pcdh18, Pcdh19, Pcdh20, or any combination thereof) and/or EVs (e.g., any of the OSN derived EVs of the present disclosure) are stamped and patterned on the surface of a device. The device can be implanted in a subject, such as where nerve regrowth is desired. In some embodiments, the one or more delta protocadherins (e.g., Pcdh1, Pcdh7, Pcdh8, Pcdh9, Pcdh10, Pcdh 11, Pcdh17, Pcdh18, Pcdh19, Pcdh20, or any combination thereof) and/or EVs (e.g., any of the OSN derived EVs of the present disclosure) are dried on the surface of the implant in the desired pattern. In some embodiments, the one or more delta protocadherins (e.g., Pcdh1, Pcdh7, Pcdh8, Pcdh9, Pcdh10, Pcdh 11, Pcdh17, Pcdh18, Pcdh19, Pcdh20, or any combination thereof) and/or EVs (e.g., any of the OSN derived EVs of the present disclosure) are patterned along with one or more polymers, such as a biocompatible polymer. In some embodiments, the polymer is a polyornithine. In some embodiments, the patterns on the device can be designed such that they form correct or desired nerve tracts. Without being bound by theory, when the device is implanted into a subject the one or more delta protocadherins (e.g., Pcdh1, Pcdh7, Pcdh8, Pcdh9, Pcdh10, Pcdh 11, Pcdh17, Pcdh18, Pcdh19, Pcdh20, or any combination thereof) and/or EVs (e.g., any of the OSN derived EVs of the present disclosure) on the device can stimulate nerve (e.g., axon) growth, synapse formation, and/or connectivity along the pattern where the one or more delta protocadherins (e.g., Pcdh1, Pcdh7, Pcdh8, Pcdh9, Pcdh10, Pcdh 11, Pcdh17, Pcdh18, Pcdh19, Pcdh20, or any combination thereof) and/or EVs (e.g., any of the OSN derived EVs of the present disclosure) are present. Without being bound by theory, nerve growth can be controlled or directed by how the one or more delta protocadherins (e.g., Pcdh1, Pcdh7, Pcdh8, Pcdh9, Pcdh10, Pcdh 11, Pcdh17, Pcdh18, Pcdh19, Pcdh20, or any combination thereof) and/or EVs (e.g., any of the OSN derived EVs of the present disclosure) are patterned on the device. In some embodiments, the devices can be used in a method of treating subject and/or promoting nerve growth by implanting them in a subject in need thereof. In some embodiments, the devices are implanted at or in proximity to a damaged or otherwise injured or dysfunctional nerve.

In some embodiments, the device is made of one or more flexible materials. In some embodiments, the device is or includes one or more meshes, stents, tubes, planar surfaces, curved members. In some embodiments, the device is or includes one or more fibrous substrates. In some embodiments, the device comprises one or more biocompatible polymers.

In some embodiments, the device is suitable for in vivo use. In some embodiments, the device is configured for cell culture use.

### Kits

Any of the compounds, compositions, formulations, particles, cells, and/or devices described herein or a combination thereof can be presented as a combination kit. As used herein, the terms “combination kit” or “kit of parts” refers to the compounds, compositions, formulations, particles, cells, and/or devices and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, and/or devices described herein or a combination thereof (e.g., agents) contained in the kit are administered simultaneously, the combination kit can contain the active agents in a single formulation, such as a pharmaceutical formulation, (e.g., a tablet, solution, suspension, powder, and/or the like described elsewhere herein) or in separate formulations. When the compounds, compositions, formulations, particles, cells, and/or devices described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, cells, and/or devices described herein or a combination thereof to a subject in need thereof.

In some embodiments, the subject in need thereof has, has had, and/or is suspected of having a nerve injury, nerve death, aberrant neuron connectivity, aberrant neuron activity, a neuropathy, or any combination thereof. In some embodiments, the subject in need thereof has, has had, and/or is suspected of having a neurodegenerative disease, disorder, and/or condition. In some embodiments, the subject in need thereof has, has had, and/or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, and/or is suspected of having a CNS neuron/nerve and/or a peripheral neuron/nerve injury, disease, disorder, and/or condition.

In some embodiments, the subject in need thereof has, has had, and/or is suspected of having an epilepsy, a seizure disease, disorder or condition, or a disease, disorder, or condition in which seizures are a symptom or result of the disease, disorder, or condition, including but not limited to non-epileptic seizures. In some embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In some embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, or is suspected of having a CNS neuron/nerve and/or a peripheral neuron/nerve injury, disease, disorder, and/or condition. In some embodiments, the disease, disorder, and/or condition is a genetic disease, disorder, and/or condition. In some embodiments, the disease, disorder, and/or condition is not a genetic disease, disorder, and/or condition.

### Methods of Using Delta Protocadherin Compositions

The delta protocadherin compositions described in the several exemplary embodiments herein and formulations thereof can be used to treat a disease, disorder, condition, and/or injury. In some embodiments, a method of treating a disease, disorder, condition, and/or injury includes administering to a subject in need thereof a population and/or an amount of the delta protocadherin compositions or formulations thereof described in greater detail elsewhere herein to the subject in need thereof.

The delta protocadherin compositions or formulations thereof described in greater detail elsewhere herein can be used to regenerate and/or enhance regeneration, growth and/or development of neurons and/or nerves in the CNS and/or periphery. In some embodiments, the delta protocadherin compositions or formulations thereof can be used to increase and/or enhance growth and/or regeneration of a neuron or nerve in the CNS and/or periphery. In some embodiments, a method of regenerating neurons and/or nerves includes administering to a subject in need thereof a population and/or an amount of one or more delta protocadherin compositions or formulations thereof described in greater detail elsewhere herein to the subject in need thereof.

In some embodiments, the delta protocadherin compositions or formulations thereof can be used to increase growth rate and/or regeneration rate of a neuron or nerve in the CNS and/or periphery. In some embodiments, delta protocadherin compositions or formulations thereof herein and formulations thereof can be used to increase growth rate and/or regeneration rate of an axon in the CNS and/or periphery. In some embodiments, a method of increasing the growth rate and/or regeneration rate of neurons and/or nerves includes administering to a subject in need thereof an amount of the delta protocadherin compositions or formulations thereof described elsewhere herein to the subject in need thereof.

The delta protocadherin compositions or formulations thereof described in the several exemplary embodiments herein and formulations thereof can be used to increase or enhance correct axon connectivity during growth and/or regeneration. This refers to the axon connecting to the appropriate target neuron or neurons during regeneration so as to be more similar to and/or like a pre-disease or pre-injured/damaged state. In some embodiments, a method of increasing and/or enhancing the correct connectivity of neurons and/or nerves, particularly the axon thereof, particularly during growth and/or regeneration, includes administering to a subject in need thereof a population and/or an amount of the delta protocadherin compositions or formulations thereof described in greater detail elsewhere herein to the subject in need thereof.

The delta protocadherin compositions or formulations thereof described in the several exemplary embodiments herein and formulations thereof can be used to improve neuron and/or axon structure and organization during growth and/or regeneration. This refers to the overall alignment, spacing, and/or positioning of the regenerating neurons so as to be more similar to and/or like a pre-disease or pre-injured/damaged state. In some embodiments, a method of improving neuron/nerve and/or axon structure and organization, synapse formation, connectivity, or any combination thereof particularly during growth and/or regeneration, includes administering to a subject in need thereof a population and/or an amount of the delta protocadherin compositions or formulations thereof described in greater detail elsewhere herein to the subject in need thereof.

In some embodiments, the subject in need thereof has, has had, and/or is suspected of having a nerve injury, nerve death, aberrant neuron connectivity, aberrant neuron activity, a neuropathy, or any combination thereof. In some embodiments, the subject in need thereof has, has had, and/or is suspected of having a neurodegenerative disease, disorder, and/or condition. In some embodiments, the subject in need thereof has, has had, and/or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, and/or is suspected of having a CNS neuron/nerve and/or a peripheral neuron/nerve injury, disease, disorder, and/or condition.

In some embodiments, the subject in need thereof has, has had, and/or is suspected of having an epilepsy, a seizure disease, disorder or condition, or a disease, disorder, or condition in which seizures are a symptom or result of the disease, disorder, or condition, including but not limited to non-epileptic seizures. In some embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In some embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, or is suspected of having a CNS neuron/nerve and/or a peripheral neuron/nerve injury, disease, disorder, and/or condition. In some embodiments, the disease, disorder, and/or condition is a genetic disease, disorder, and/or condition. In some embodiments, the disease, disorder, and/or condition is not a genetic disease, disorder, and/or condition.

Administration can be hourly, daily, weekly, monthly, or yearly. Administration can be one or more times an hour, one or more times a day, one or more times a week, one or more times a month, or one or more times a year. Administration can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more times as appropriate per hour, day, week, month and/or, year.

In some embodiments, the composition containing the delta protocadherin compositions or formulations thereof described elsewhere herein can be administered long with a co-therapy. The co-therapy can be administered in the same formulation as the delta protocadherin composition, at substantially the same time, or sequentially to the composition and/or formulation containing the OSN EVs. In some embodiments, the agent(s) in addition to the delta protocadherin compositions or formulations thereof in the co-therapy can treat and or prevent a disease or symptom thereof. In some embodiments the co-therapy is optionally a polynucleotide, a polypeptide, a nutrient (e.g., lipid, amino acid, carbohydrate, peptide, protein, sugar, vitamin, mineral, and/or the like), genetic modifying system or component thereof, antibody or fragment thereof, aptamer, affibody, small molecule chemical agent (e.g., a therapeutic and/or prevention), an immunomodulator, a hormone, an antipyretic, an anxiolytic, an antipsychotic, an analgesic, an antispasmodic, an anti-inflammatory agent, an anti-epileptic agent, an anti-histamine, a growth factor, an anti-infective, a radiation sensitizer, a chemotherapeutic, a neurotransmitter antagonist, a neurotransmitter agonist, or any combination thereof.

Administration of the delta protocadherin compositions or formulations thereof and/or any co-therapy can be by any suitable administration routes and/or methods. Exemplary suitable administration routes and/or methods are described in greater detail elsewhere herein.

In some embodiments, the delta protocadherin compositions or formulations thereof of the present disclosure are used in vitro, in vivo, or ex vivo to promote stem cell division. In some embodiments, the stem cells are pluripotent stem cells. In some embodiments, the stem cells are totipotent stem cells. In some embodiments, the stem cells are multi-potent stem cells. In some embodiments, the stem cells are olfactory epithelium stem cells. In some embodiments, the stem cells are olfactory neuron stem cells. In some embodiments, the stem cells are epithelial stem cells.

In some embodiments, the delta protocadherin compositions or formulations thereof of the present disclosure can be used in vitro, in vivo, or ex vivo to promote cell reprogramming. In some embodiments, the delta protocadherin compositions or formulations thereof of the present disclosure can be used to reprogram cells to produce induced pluripotent stem cells. In some embodiments, differentiated or somatic cells are cultured in the presence of olfactory neuron derived EVs and/or delta protocadherins of the present disclosure.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Olfactory Sensory Neuron Derived EVs

Peripheral neuropathy involves damage to neurons in the peripheral nervous system (PNS), and has numerous causes, including: traumatic injury, infection, metabolic disorder, exposure to environmental toxins, genetic causes, and others. For example, injury can be caused by damage during surgery, vehicular collision, or prolonged immobility. Recovery time depends on the extent of damage, and regeneration can be as slow as 1-2 mm per day. Critically, there is no treatment to accelerate this regrowth (1,2).

Current approaches to repair neuronal damage include microsurgery, glues to promote regrowth, and the development of conduits to bridge the proximal and distal stump (3,4). Another approach is to use cell-based therapies. In particular, the use of Schwann cells (a form of glia) is the most commonly studied such therapy (5). Schwann cells enwrap axons, providing neurotrophic factors to support axon regrowth, myelin to protect and insulate axons, and modulate the immune response. However, they proliferate slowly and are difficult to obtain. Alternatives have included stem cells isolated from various sources, which are then differentiated into Schwann-cell like cells.

More recently, efforts aimed at identifying the factors produced by Schwann cells to promote regrowth suggest that extracellular vesicles (EVs) are important for this process. EVs are secreted by all cell-types, and contain RNAs, DNA, and protein. They have recently been identified as an important form of cell-cell communication. Uptake of EVs by cells can lead to significant changes in cellular homeostasis.

Schwann cell derived EVs were shown to promote the regrowth of peripheral neurons of neurons grown in culture and in rodents with sciatic nerve injury (6). Other groups have also shown that EVs derived from olfactory ensheathing cells (another cell-type similar to Schwann cells and are also glial-like) also can promote regrowth of neurons in culture and in surgical models (7). These results show glial culture-derived EVs are an important mechanism for promoting axonal regrowth both in culture and in vivo.

As noted above, the role of Schwann cells in promoting axonal regrowth has been extensively studied, and EVs derived from Schwann cells promote regrowth. In contrast, the role of neuron culture-derived EVs in axon growth has only been studied in a single paper (8). In this study, cortical neuron culture-derived EVs inhibited growth of neurons grown in culture, and did not enhance regrowth. Further, as far as we are aware, there are no papers on neuronal-derived EVs on regeneration in vivo.

A unique feature of this disclosure is the use of olfactory neuron culture-derived EVs to promote axon growth. As noted above, almost all efforts on promoting axonal growth have focused on glia, and glial-derived EVs. This is based on decades of study showing the importance of glia in promoting neuronal survival and function. However, a major area of research that surprisingly has not been studied is the role of the neurons themselves in this process. In particular, we focused on olfactory neurons because they are well-known to regenerate constantly throughout the life of the animal.

Applicant theorized that this regrowth confers unusual properties to olfactory neurons as compared with cortical neurons (which do not regenerate). Applicant therefore isolated EVs from cultured olfactory neurons. The initial results show these neuronal culture-derived EVs, in contrast to the prior study on cortical neurons, do in fact promote neuronal regrowth. Applicant demonstrated this using two model systems, applying olfactory neuron culture-derived EVs to cultures of olfactory neurons (to assess the effects of adding additional EVs to olfactory neurons), and also to cultures of dorsal root ganglia (DRG, a commonly studied peripheral neuronal type but clearly distinct from olfactory neurons). For example, in FIG. 1A-1B, Applicant quantitated neurite length of DRGs grown in culture with and without the addition of olfactory neuron culture-derived EVs. The EVs clearly increase the number of DRGs with increased neurite length, demonstrating an effect on DRG growth. Thus, olfactory neuron culture-derived EVs can enhance the growth not only of olfactory neurons, but also of a distinct neuronal cell-type.

### Purification of Olfactory EVs

Purification of EVs followed published procedures. Briefly, EV purification involved dissecting olfactory epithelia from mice. Mice were of varying age, but typically were young postnatal animals (postnatal day 6 to 10). Epithelia recovered were enzymatically digested and then plated on coverslips coated with an appropriate adhesive, such as poly ornithine. An appropriate neuronal culture media (typically BrainPhys supplemented with SM1 and G418) was then added and neurons are cultured for about 2 days. The supernatant from multiple samples is collected and pooled. EVs are isolated by using a well-established centrifugation protocol involving sequential low-speed spins followed by a high speed (100,000×g) to pellet EVs. EVs were resuspended in PBS and frozen prior to use.

The EVs can be isolated from olfactory neurons isolated from different ages of the subject's life. Without being bound by theory, it is believed embryonic EVs may be better than adult EVs at promoting growth, or adult EVs might be better because they are derived from regenerating neurons.

The olfactory neuron culture-derived EVs can be used to help enhance peripheral neuron regrowth in both humans and animals. They can be incorporated into hydrogels, used during surgery to promote repair, injected systemically to enhance growth, and could be inhaled as well. It is also possible, because olfactory neurons cross into the central nervous system, that they may also enhance regeneration of central nervous system neurons. Thus, they could also promote regeneration of neurons affected by degenerative diseases such as Alzheimer's and Parkinson's. Finally, because EVs are potential vehicles for drug delivery, studies have been performed showing EVs derived from human HEK293 cells do not appear to induce an immune response when injected into mice. Thus, it is possible that EVs from olfactory neurons in mice (which can be generated rapidly) can be applied to other species.

### REFERENCES RELATED TO EXAMPLE 1

1. Pfister B J, Gordon T, Loverde J R, et al. Biomedical engineering strategies for peripheral nerve repair: Surgical applications, state of the art, and future challenges. Critical Reviews in Biomedical Engineering. 2011; 39(2):81-124.
2. Takahashi K., Yamanaka S. Induction of pluripotent stem cells from mouse embryonic and adult fibroblast cultures by defined factors. Cell. 2006; 126:663-676.
3. Xue, W., Shi, W., Kong, Y., Kuss, M., and Duan, B. Anisotropic scaffolds for peripheral nerve and spinal cord regeneration. Bioact. Mater., 2021, 6(11): 4141-60.
4. https://www.intechopen.com/chapters/55127.
5. Hood, B., Levene, H. B., Levi, A. D. Transplantation of autologous Schwann cells for the repair of segmental peripheral nerve defects, Neurosurg. Focus, 2009 26:1-9.
6. Lopez-Verilli, M. A., Picou, F., Court, F. A. Schwann cell-derived exosomes enhance axonal regeneration in the peripheral nervous system Glia, 2013. 61(11):1795-806.
7. Xia, B, Gao, J. Li, S. et al. Extracellular Vesicles Derived From Olfactory Ensheathing Cells Promote Peripheral Nerve Regeneration in Rats. Front. Cell. eurosci., 2019, 13, Article 548.
8. Gong, J., Korner, R., Gaitanos, L., Klein, R. Exosomes mediate cell contact-independent ephrin-Eph signaling during axon guidance. Journal of Cell Biol., 2016, 214(1): 35-44.

### Example 2—Effects of Extracellular Vesicles on Neuronal Regeneration in the Olfactory System

Canine spinal cord injuries are 2% of cases presented to veterinarians. Acute spinal cord injury has a poor prognosis and surgery must occur within 24 hours of injury but is highly invasive and variably successful. A less invasive method that has proven successful is the injection of olfactory ensheathing cells into the injured spines of dogs. How olfactory ensheathing cells, a type of glial cell, produce this effect is not currently understood. The olfactory nervous system is the most exposed group of neurons to the exterior environment leading to the most frequent damage and the necessity of regeneration. While adult neurons in the CNS and parts of the PNS have limited capability to regenerate post damage, olfactory sensory neurons (OSN) regenerate every ˜30 days. Further, past studies have preliminarily found that Schwann cell derived extracellular vesicles (EVs) injected in vivo improved the axonal regrowth of mechanically damaged dorsal root ganglia. EVs, which are responsible for intercellular communication, are created by exocytosis from the lipid bilayer and can contain cytosolic proteins, membrane proteins, mRNAs, noncoding RNAs, and even DNA. Within the olfactory system, EVs are produced by OSN and surrounding glial cells, keeping the two cell types in communication. We hypothesized that OSN EVs would have an effect on the rate and accuracy of neuronal regeneration in damaged olfactory systems in vivo. OSN were destroyed using methimazole (IP), a compound that induces the degeneration of the OSN in the epithelia while maintaining the integrity of the lamina propria and cribriform plate. EV injections were tested at 2, 4, and 8 μg dosages, and mice OSN regrowth was examined at days 0, 14, 28, and 42.

### Example 3—Determining the Effect of Neuron-Derived Extracellular Vesicles on Axonal Regeneration of Dorsal Root Ganglia

Peripheral nerve diseases such as traumatic neuropathies and sensory neuropathies have been found in dogs and cats. A problem in this field is finding treatments for nerves that can slow the progress of these diseases. Nerve growth factor has been shown to promote the initial sprouting of axons, but other factors that help guide axons to their appropriate targets are still poorly characterized. Studies with Schwann- and olfactory ensheathing cell-derived extracellular vesicles (EVs) have been shown to promote axonal regeneration after nerve damage. This study tested whether other neuron-derived EVs also promote the growth of dorsal root ganglia (DRG) axons. This was accomplished by isolating individual DRG neurons from mice and culturing them with or without neuron-derived EVs. Additionally, whole DRGs were plated and treated with or without EVs. The DRG neurons and whole DRGs were stained with beta-tubulin antibody to detect neurite growth, and then analyzed by comparing axonal length between EV treated and control conditions. The application of neuron-derived EVs may provide a starting point for developing a treatment that can delay the progression of peripheral nerve diseases.

### Example 4—Regeneration in the Mouse Peripheral Nervous System

There are approximately 12,500 new cases of spinal cord injuries per year. 38 percent are caused by vehicular crashes and about 30 percent are due to falls. Approximately 340,000 currently living with a spinal cord injury. The average lifetime costs associated directly or indirectly with a spinal cord injury range from about $428,000 to $1.4 million, based on severity.

Olfactory neurons regenerate constantly through life with neurons being born approximately every 30 days. Newly born olfactory neurons must find their way back to their proper targets in the olfactory bulb in the brain. Granger et al., 2012 demonstrated spinal cord regeneration in dogs in a randomized controlled trial at Cambridge Veterinary Hospital. To do this they removed a special type of cell called the olfactory ensheathing cell (OEC), a type of glial cell, from the nasal passageways of the dogs, grew them in culture until a sufficient number had been produced, and then transplanted them at the site of injury. Tabakow et al. (2014, Cell Transplant., 23, 1631-1655) demonstrated functional regeneration of supraspinal connections in a patient with a transected spinal cord following transplantation of bulbar olfactory ensheathing cells. Applicant assessed whether the regenerative properties of the olfactory system neurons can be used to repair damage in the CNS and/or PNS.

Extracellular vesicles, including exosomes and microvesicles, are small, nano-to-micrometer vesicles that are released from cells.

Paolicelli et al. (Neuroscience, 405(1) (2019) summarized cell-to-cell communication by extracellular vesicles between neurons, astrocytes, and microglia within the central nervous system. See e.g., Paolicelli et al. at FIG. 1. However, there is no understanding to Applicants of effect of olfactory neuron derived EVs in the peripheral nervous system.

Without being bound by theory, Applicant tested two hypotheses: (1) OEG-derived EVs promote OSN regeneration; and (2) OSN-derived EVs do not promote OSN regeneration. Briefly, “control” OSN derived EVs were generated by culturing primary olfactory neurons (FIG. 2A). Neuronal EVs were then purified (FIG. 2B). Purified olfactory neuron EVs were then applied to cultured OSNs. Total outgrowth, longest branch length, and neurites were evaluated (FIG. 2C-2G). Based on the hypotheses made, the results were unexpected. It was observed that OSN-derived EVs do promote OSN regeneration.

To examine the effect of OSN-derived EVs on in vivo regeneration Applicant destroyed the olfactory sensory neuron layer in mice via methimazole on Day 0. Ablation by methimazole forces regeneration of olfactory cells. On days 5, 12, 19, 26, 33, and/or 40 post day 0, OSN-derived EVs were delivered to the mice intranasally at varying amounts. Saline was and methimazole (MZ or MI) in saline were delivered as controls. The amount of EVs delivered in a saline carrier were 2 μg, 4 μg, or 8 μg. As shown in FIG. 3, OSNs form a layer within the olfactory epithelium. Sustentacular cells provide various support functions for the olfactory epithelium and neurons.

Applicant evaluated neuronal regeneration for correct and incorrect regeneration to the olfactory bulbs. See FIG. 4A-4B. Mombaerts, Cell. 87(4):675-686 (1996) provided an olfactory sensory map demonstrating correct neural pathways from the nasal epithelium to the olfactory bulb. See e.g., Momaerts. 1996. at e.g., FIG. 2A-2B.

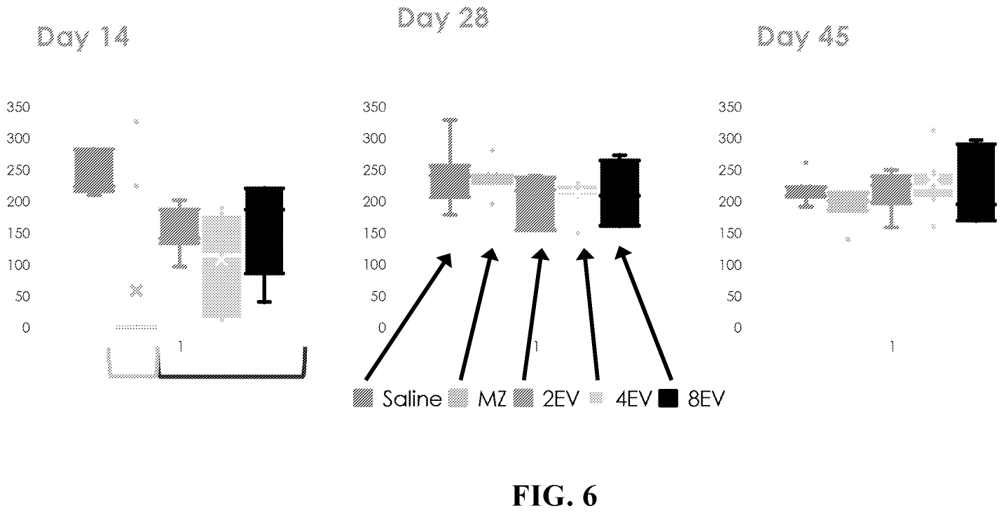

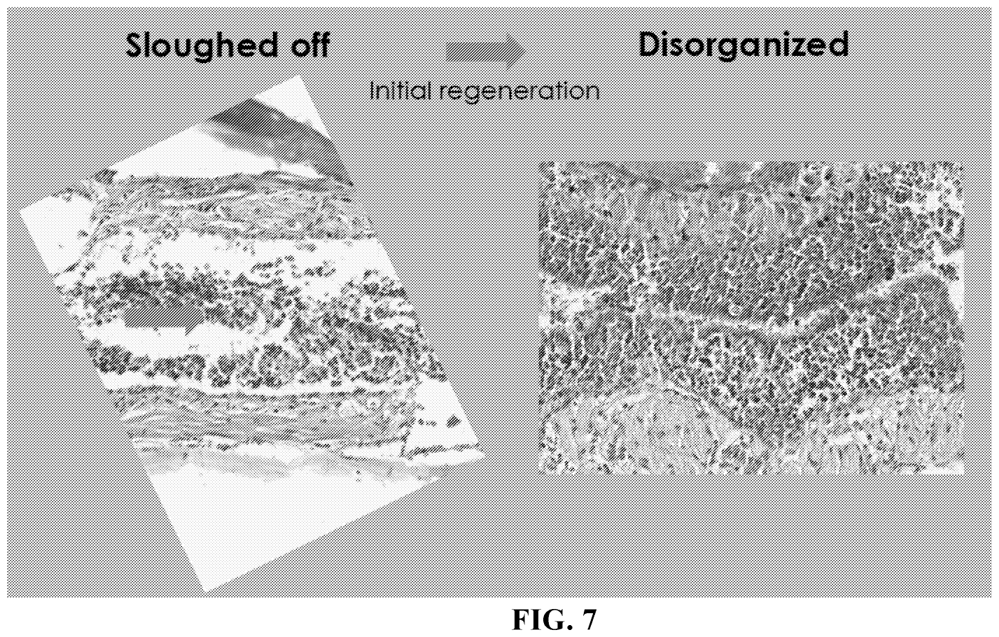

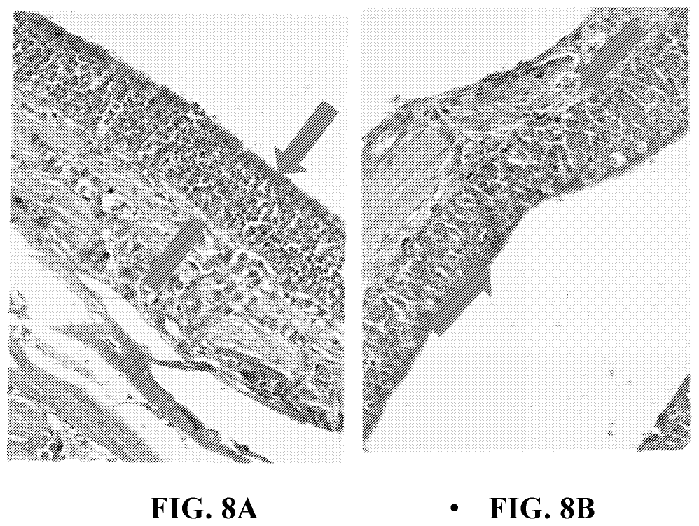

FIG. 5A-5B show fluorescent and stained microscopic images in saline (FIG. 5A) and methimazole ablated (FIG. 5B) olfactory epithelium. As shown in FIG. 6, OSN derived EVs promoted epithelial regeneration. As shown in FIG. 7, methimazole caused sloughing and the initial regeneration was disorganized. FIG. 8 shows the regeneration of the olfactory epithelium and neurons in saline control and EV treated mice.

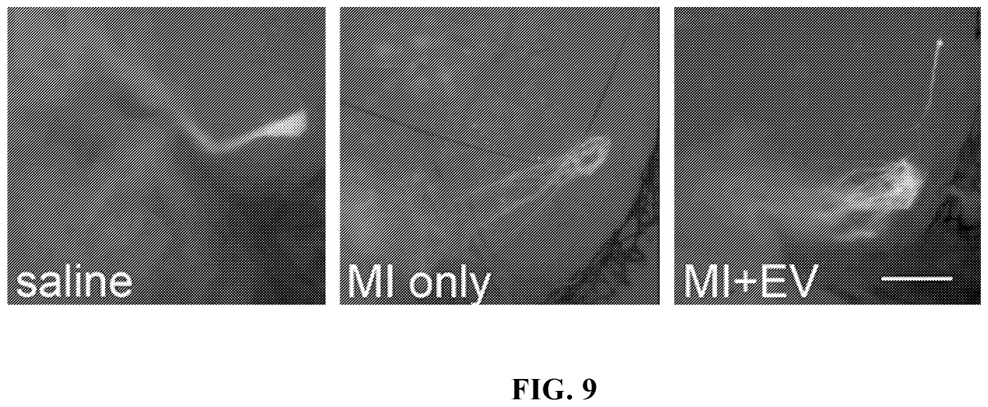

FIG. 9 shows glomeruli in saline control, MI only control, and EV treated groups at day 21 after ablation.

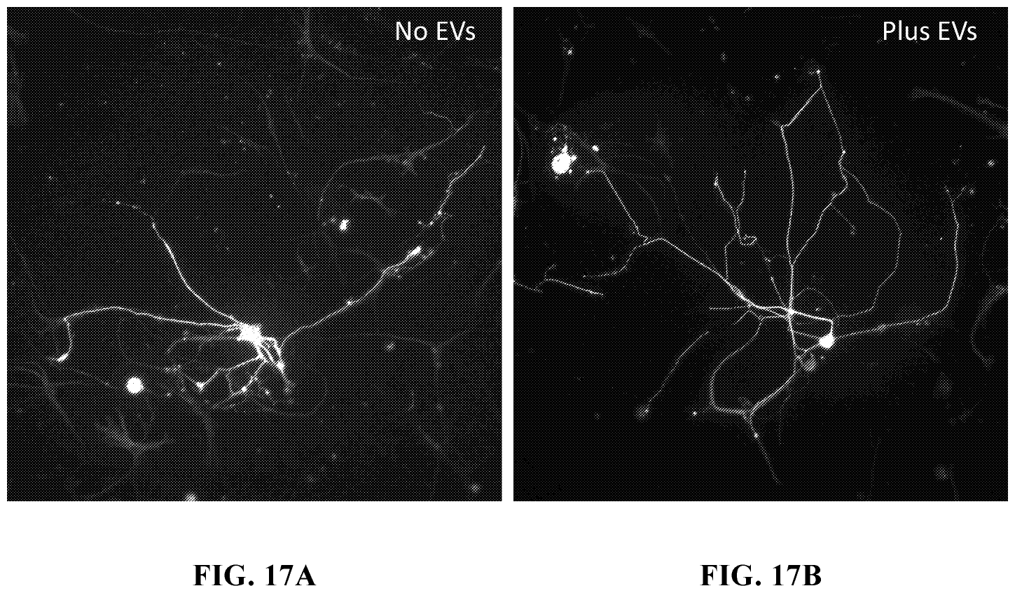

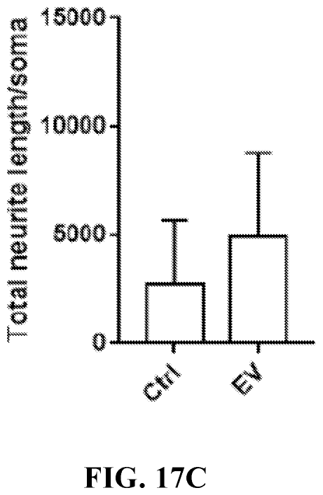

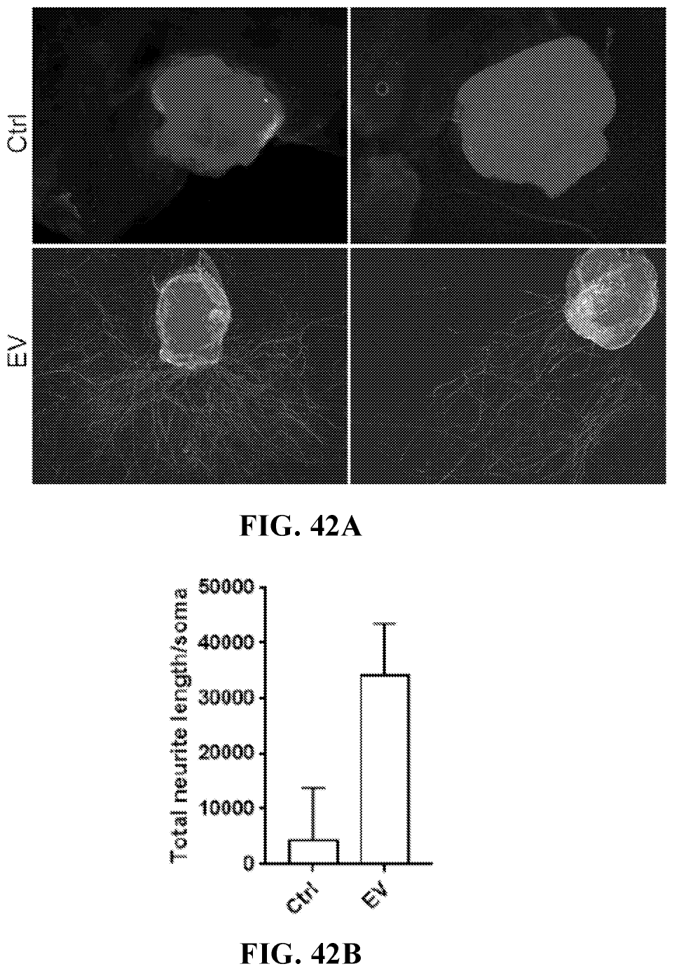

As shown in FIG. 17A-17C EVs from cultured OSNs can increase the total area of neurites in regenerating dorsal root ganglion (DRG) neurons. Further, in cultured DRGs, treatment with OSN derived EVs increased neurite length (FIG. 42A-42B).

In general, in the epithelial cells at Day 14 post MZ, EV treated group was more improved than the MZ only group. Variable morphologies were observed. Glomeruli were undefined in all groups at Day 14. At Day 28 all conditions were healthy. At day 28 the group treated with 8 μg EVs had the healthiest glomeruli. At Day 45 all conditions were healthy. At day 45 the group all glomeruli were healthy, with the 4 μg and 8 μg EV treated groups having the most defined glomeruli.

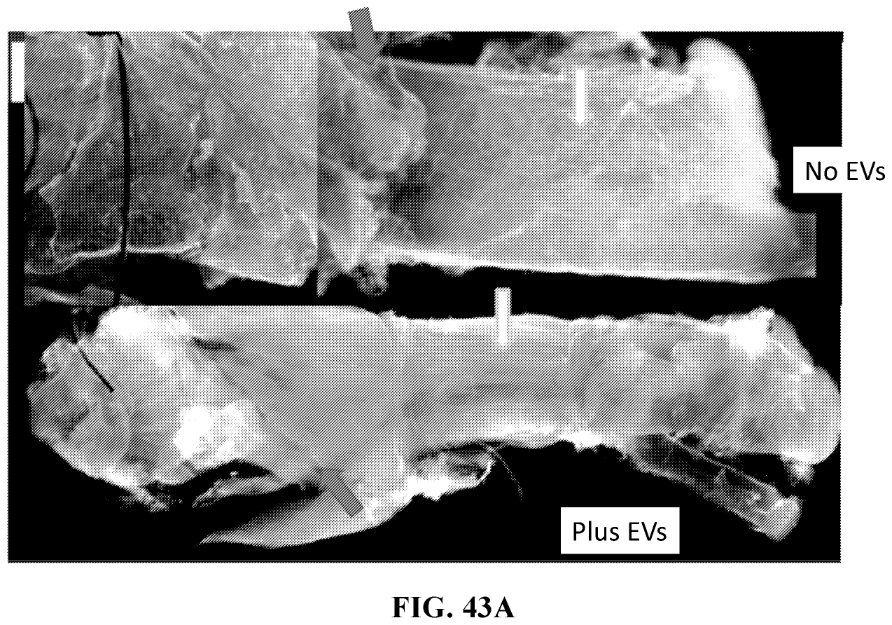

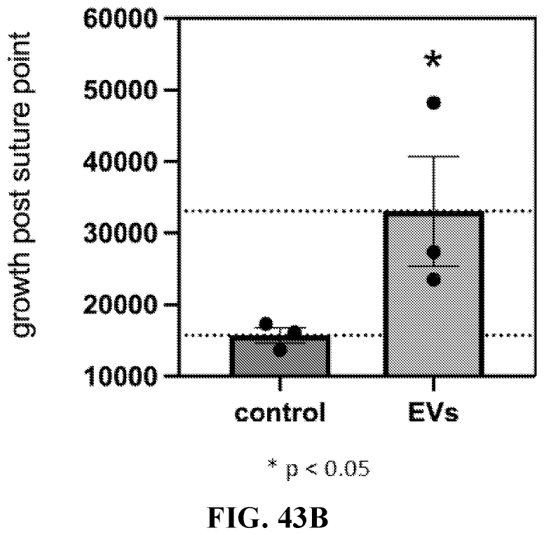

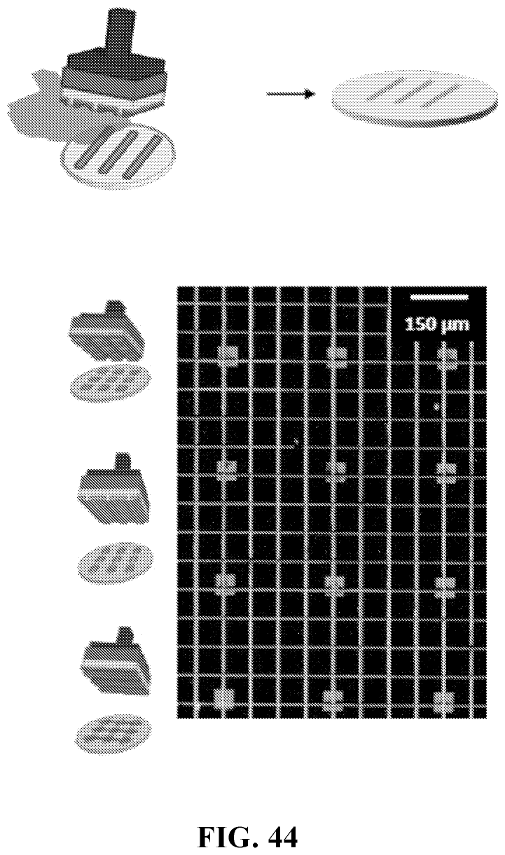

EVs from cultured OSNs were examined for their ability to improve peripheral nerve regrowth after injury. Applicant injured the sciatic nerve according to a protocol described in Niemi et al., 2020. Methods In Molecular Biology Protocols: Axon Degeneration. DOI: 10.1007/978-1-0716-0585-1_16. OSN EVs were delivered to the point of injury and as shown in FIGS. 18A-18B and 43A, treatment OSN EVs improved the organization of the axons, which is indicative of improved neuron regeneration of a peripheral nerve. Further, treatment with EVs increased neuron growth post suture point as compared to the control (FIG. 43B).

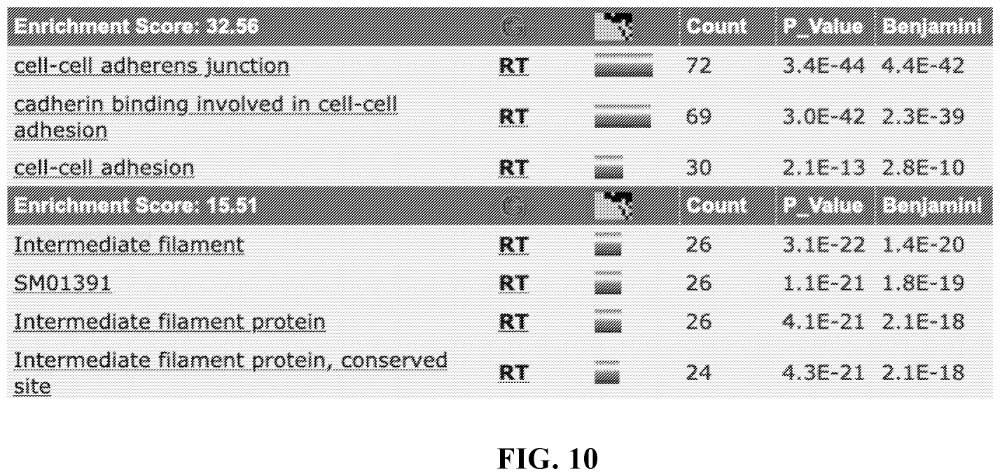

Applicant investigated molecules that promoted OSN regeneration. As shown in FIG. 10, Nano LC MS/MS returned 559 proteins that appeared involved. LC MS/MS results also demonstrated that the signal transduction pathway for delta protocadherins was also over-represented.

Spatial patterns of gene expression in the olfactory bulb have been examined. See e.g., Lin et al., PNAS. 2004. 101 (34) 12718-12723. Williams et al., Front. Neuroscience 2007. Evaluated expression of protocadherins and Bisogni et al., eLife. 2018. evaluated delta protocadherins expression in EVs.

Protocadherin (Pcdh19) is unusual in that it is not a channel protein and that it is X-linked, but only heterozygous females are generally affected. A mutation in Pcdh19 is the cause of female limited epilepsy. There are also comorbidities of hyperactivity. Autism, and obsessive compulsive disorder. Delta protocadherins mediate the promotion of regeneration by OSN-derived EVs.

A CRISPR mouse mutant recreation of known nonsense mutations in Pcdh19 was generated. MRI in humans showed axon tract defects. However, none were identified in mouse models. To date, although learning defects have been observed in mice, no epileptic phenotypes have been observed in mice.

Applicant isolated EVs as previously described (e.g, FIGS. 2A-2B) and purified olfactory neuron EVs were then applied to cultured OSNs. Average neurite length was the same between control and protocadherin 19^{-/-} treated mice.

### Example 5—Effects of Extracellular Vesicles (EVs) on Neuronal Regeneration in the Olfactory System

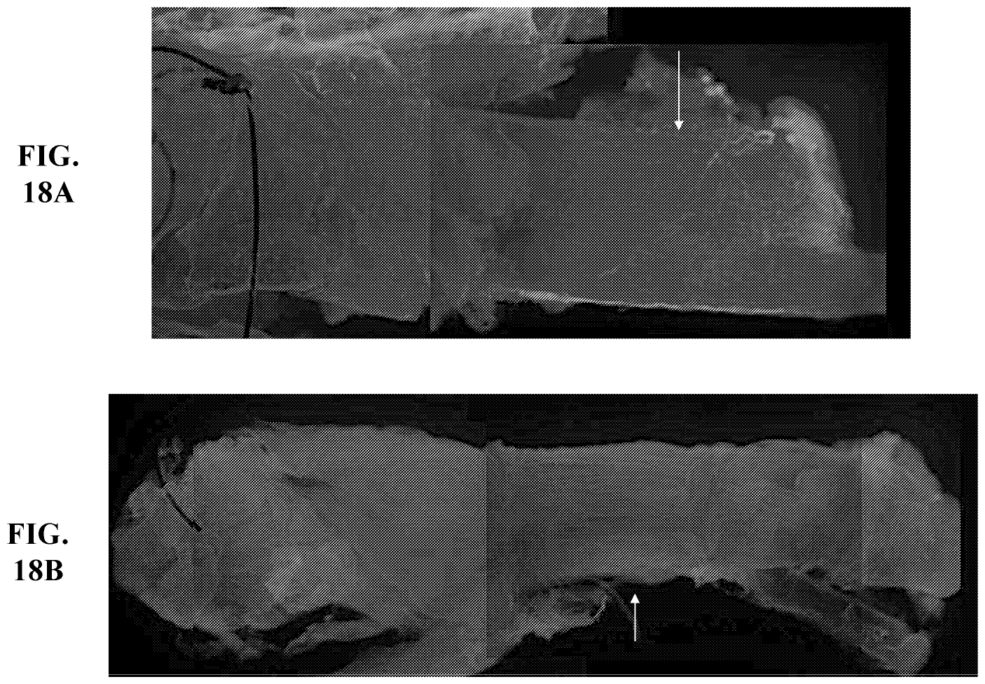

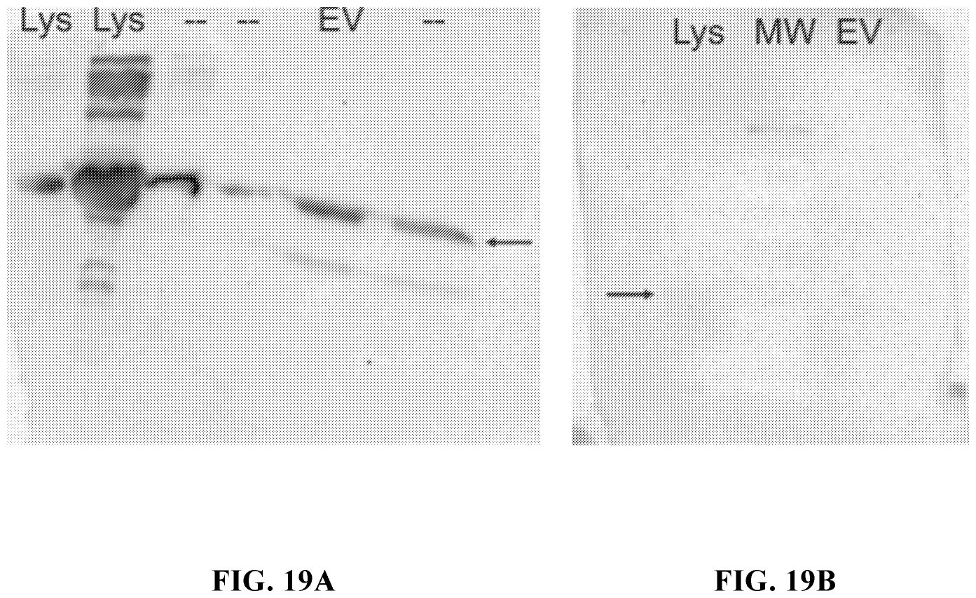

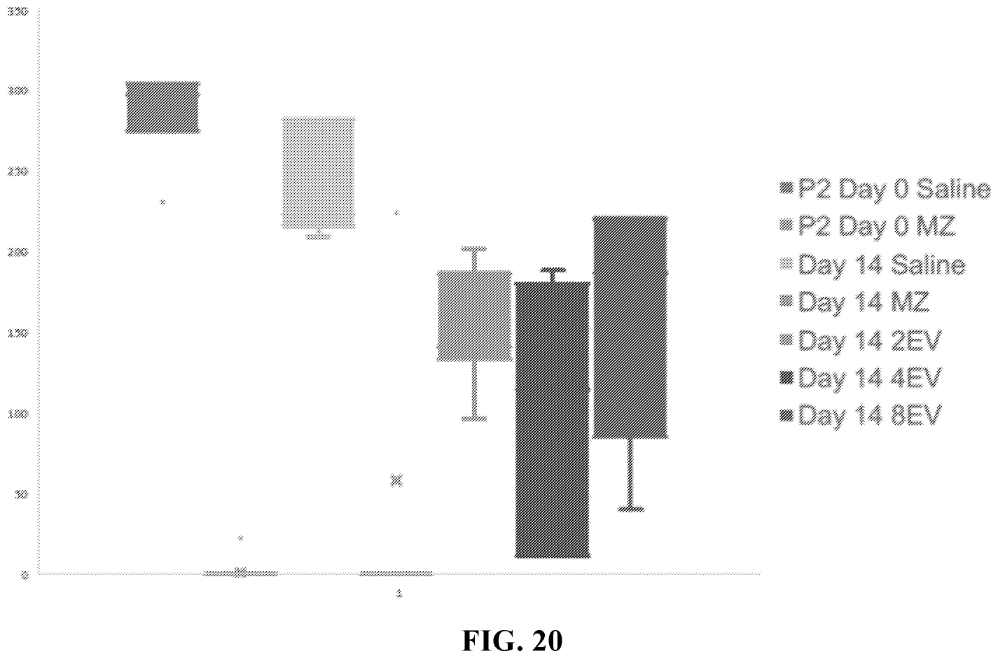

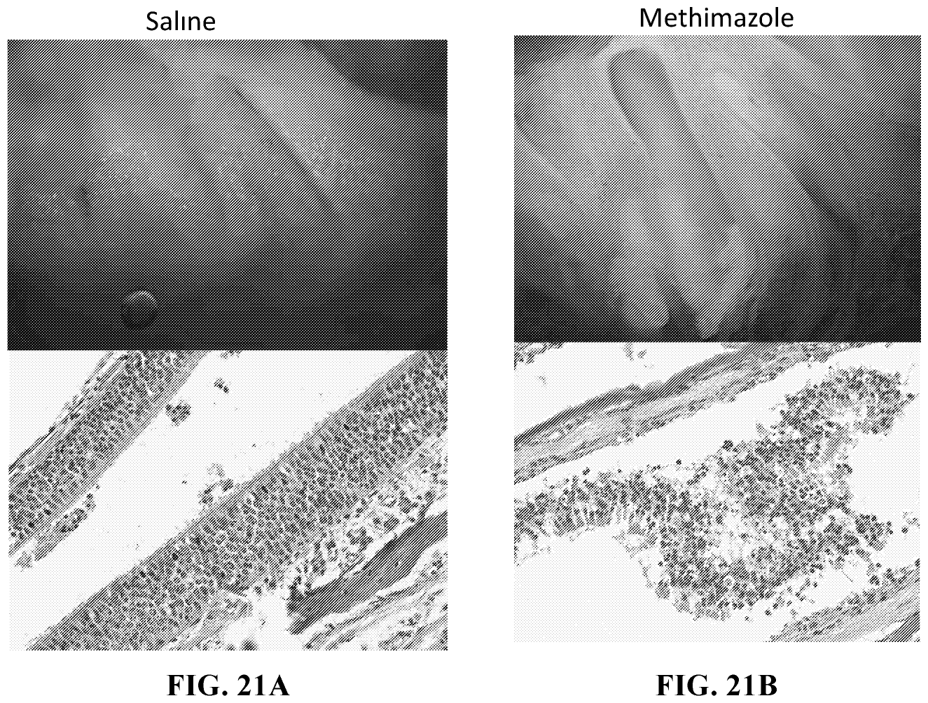

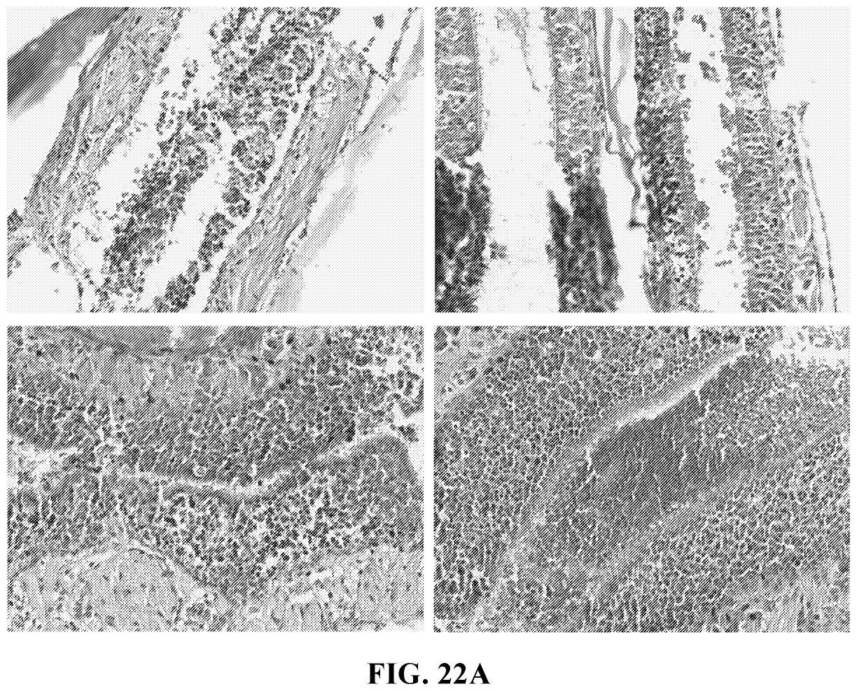

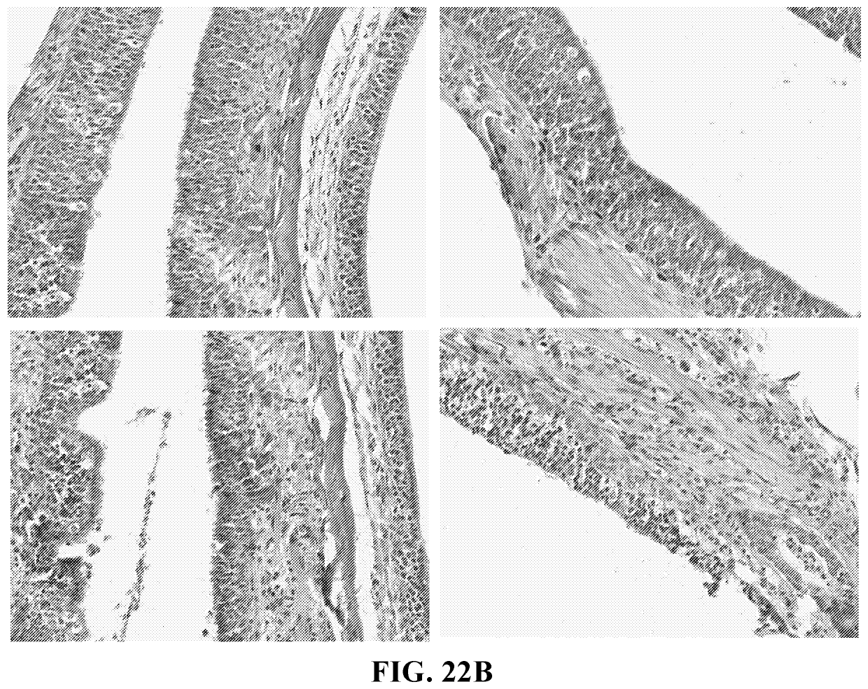

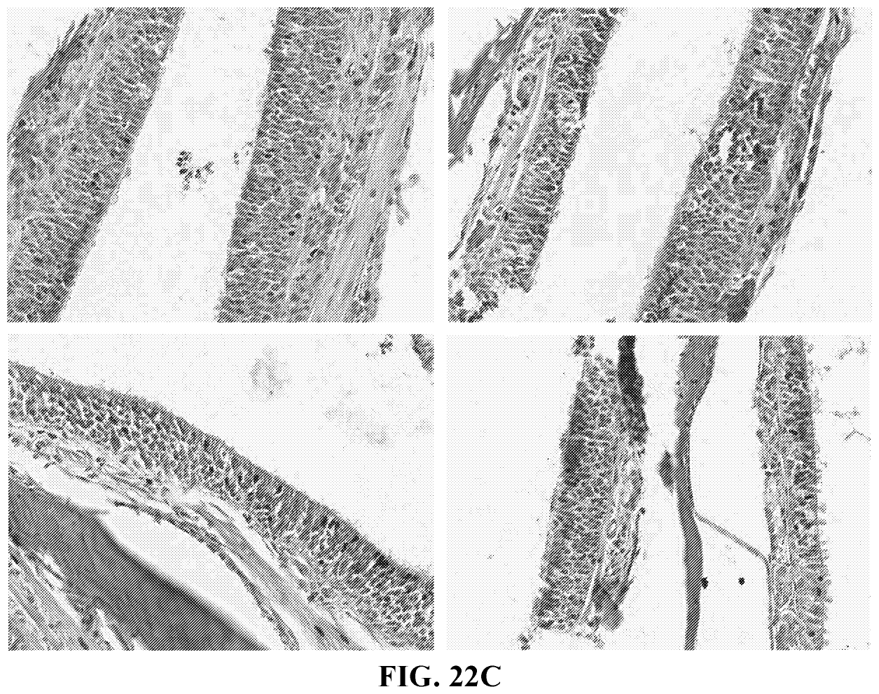

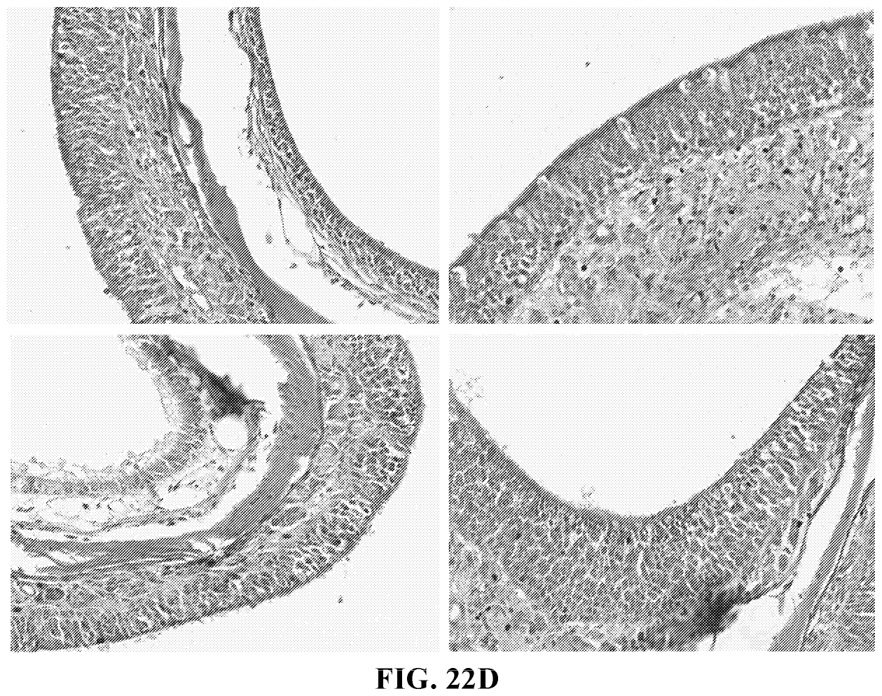

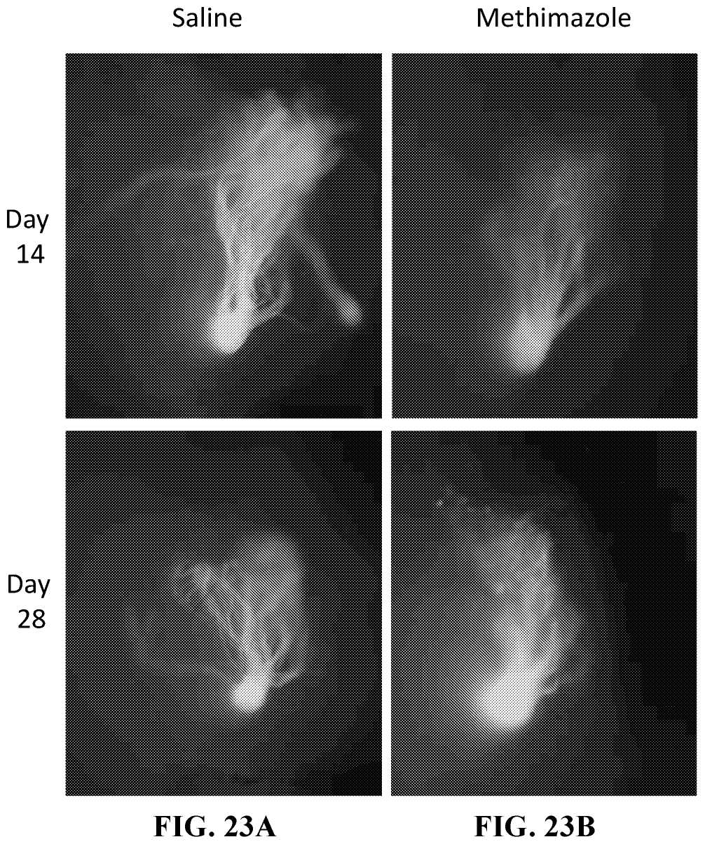

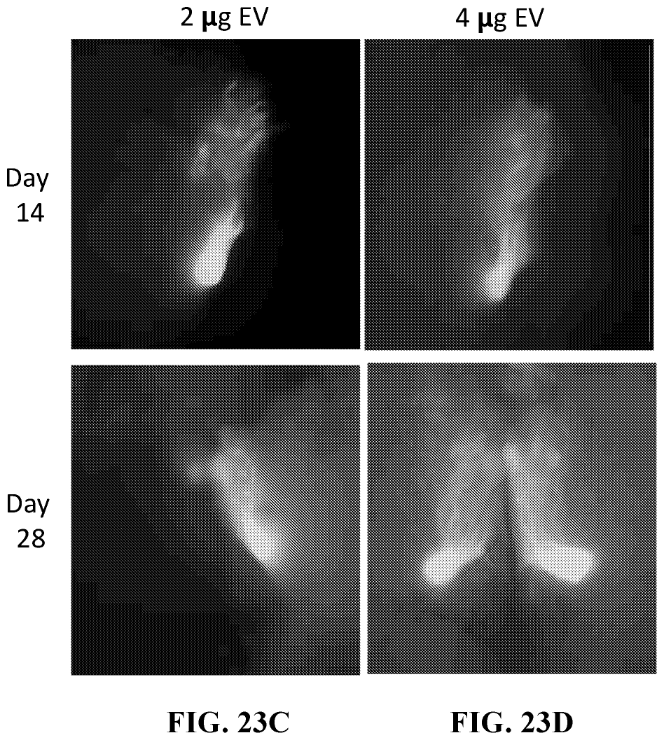

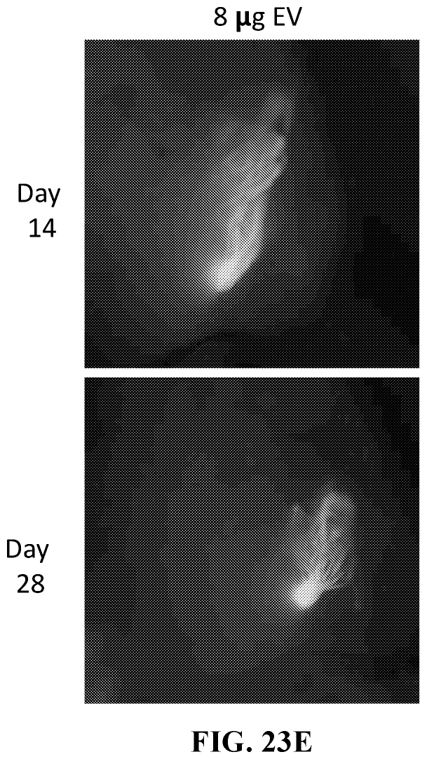

EV stock collected from olfactory sensory neurons was successfully purified as shown in FIG. 19A-19B. The OSN layer was ablated using MZ and EVs were administered to ablated tissue as previously described in Example 4. The OSN layer for each condition's epithelium was measured at five randomized locations and compared. See FIG. 20. FIG. 21A-21B shows fluorescent and stained microscopic images of the olfactory epithelium at Day 0. FIG. 22A-22D show hematoxylin and eosin staining of the olfactory epithelium of the different treatment groups at Day 14. FIG. 23A-23E shows GFP imaging of OSN axons at Day 14 and Day 28 across the treatment groups.

### Example 6—Role of Extracellular Vesicles in Regeneration

Neurodegenerative diseases and conditions are subjects of considerable research due to the difficulties that exist in achieving functional recovery. During embryonic neurogenesis, the developing neuron can form interconnected and strong networks; however, in adulthood, central nervous system (CNS) neurons lose their ability to self-repair and regenerate. The elucidation of novel proteins and mRNA responsible for neuroregeneration is crucial for developing treatments that target neurodegenerative diseases. The peripheral nervous system (PNS) repairs and regenerates throughout life, and thus is a key model for investigating this. Olfactory sensory neurons (OSNs), especially, represent a promising avenue for studying regeneration due to their remarkable regenerative capabilities, possessing the ability to not only extend growth and development but also to continuously regenerate and reestablish connections with the olfactory bulb regardless of injury. These results in the OSNs of mice suggest that OSN-derived extracellular vesicles (EVs) promote neuronal growth. Moreover, Applicant characterized the effects on regeneration from different subtypes of EVs. Finally, Applicant validated a method for manipulating EV content and identified specific proteins that may be important for growth and regeneration. The discovery of important regenerative-inducing factors in the brain will ultimately open new therapeutic avenues for the intervention of neurodegenerative diseases.

### Methods

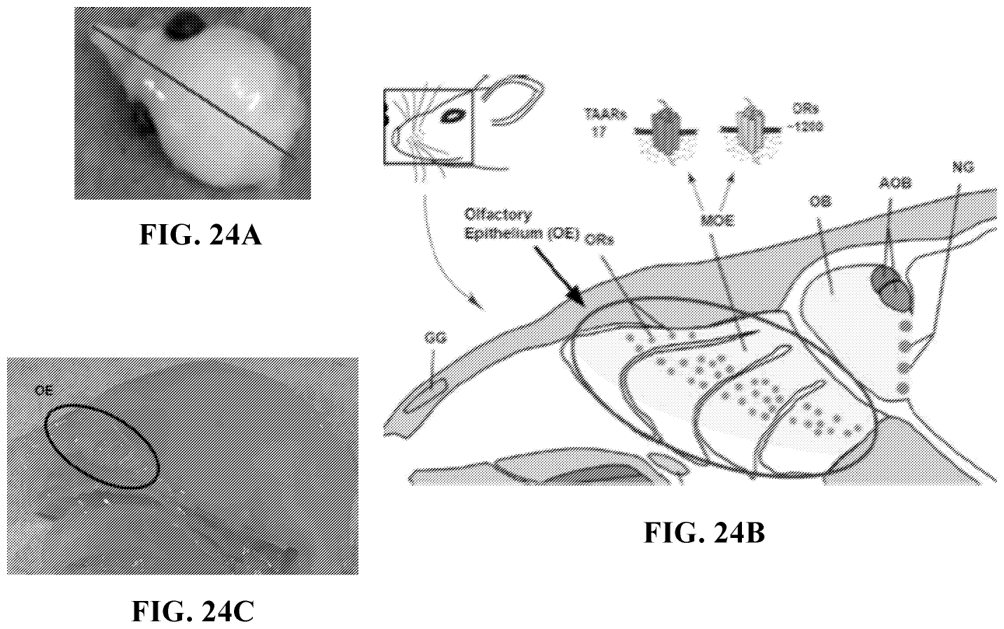

As shown in FIG. 24A-24C, the olfactory epithelium in mice was dissected and isolated. FIG. 24A shows a dorsal view of a neonatal mouse head; line depicts the intended cut through the midsagittal plane. FIG. 24B shows a schematic representation of the mouse olfactory system. FIG. 24C shows a sagittal view of an opened nasal cavity.

FIG. 25A-25C shows the data analysis apparatus of immunostained sample images of Neurons and ImageJ software for automated analysis of neurons. Applicant analyzed immunostained images (FIG. 25A) via ImageJ software which automatically traces neurons based on interpretation of where cell bodies and neurites are located. (FIG. 25B) shows the input and (FIG. 25C) shows the output, which ultimately converts into an excel sheet for statistical analysis.

### Results

OSN-Derived EVs were Successfully Purified and Validated

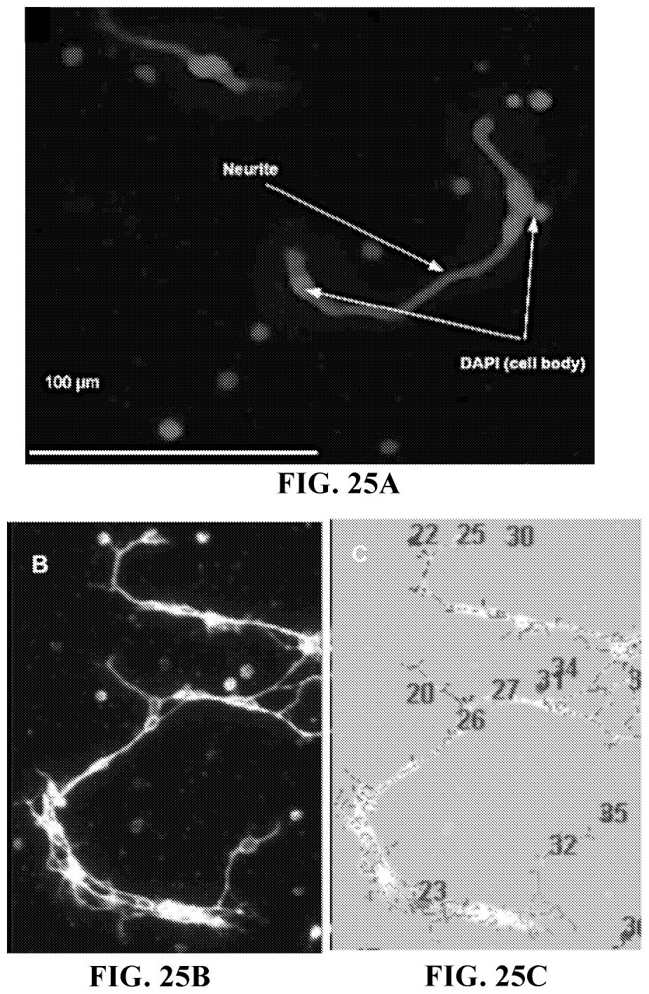

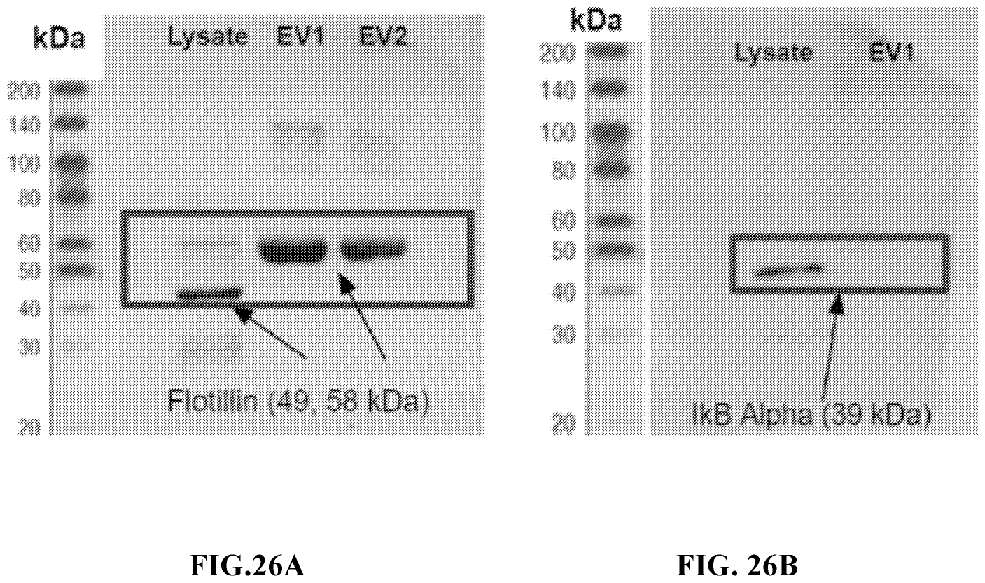

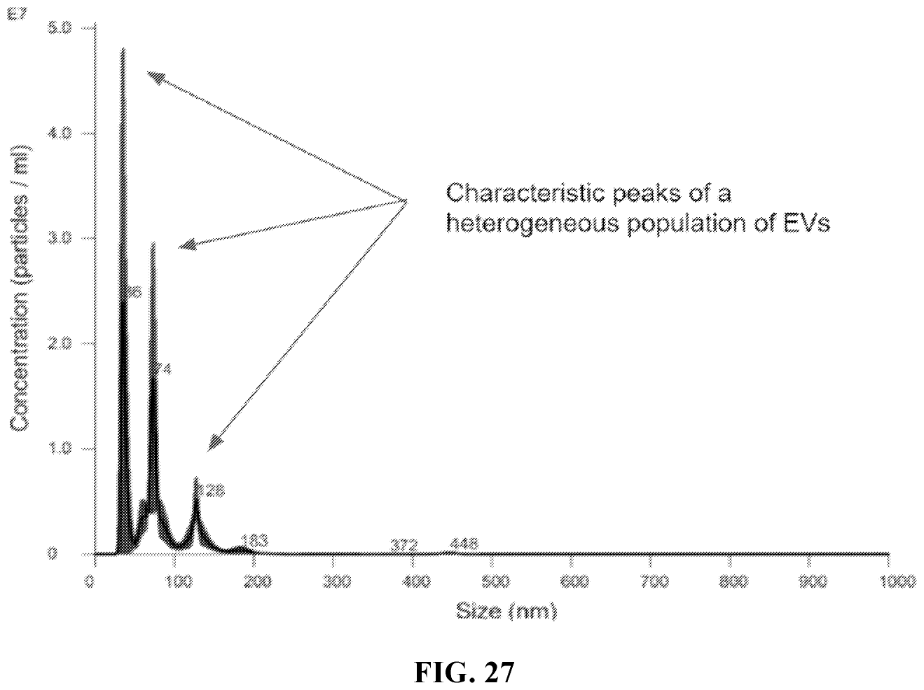

FIG. 26A-FIG. 26B shows a representative western blot for verification of EV Purification. FIG. 26A shows a western blot that demonstrates exposure of lysate and two EV samples to flotillin-2 primary antibody. FIG. 26B shows a western blot that shows exposure of lysate and one EV sample to IkB alpha primary antibody. FIG. 27 shows Nanosight NS300 nanoparticle analysis for secondary verification of EV purification. There are characteristic peaks (36 nm, 74 nm, and 128 nm) as well as smaller ones (183 nm, 372 nm, and 448 nm) that are all within expected ranges in a heterogenous population of EVs (both exosomes and microvesicles).

### Overexpression of OSN-Derived EVs Significantly Promote Growth in OSN Cultures In Vitro

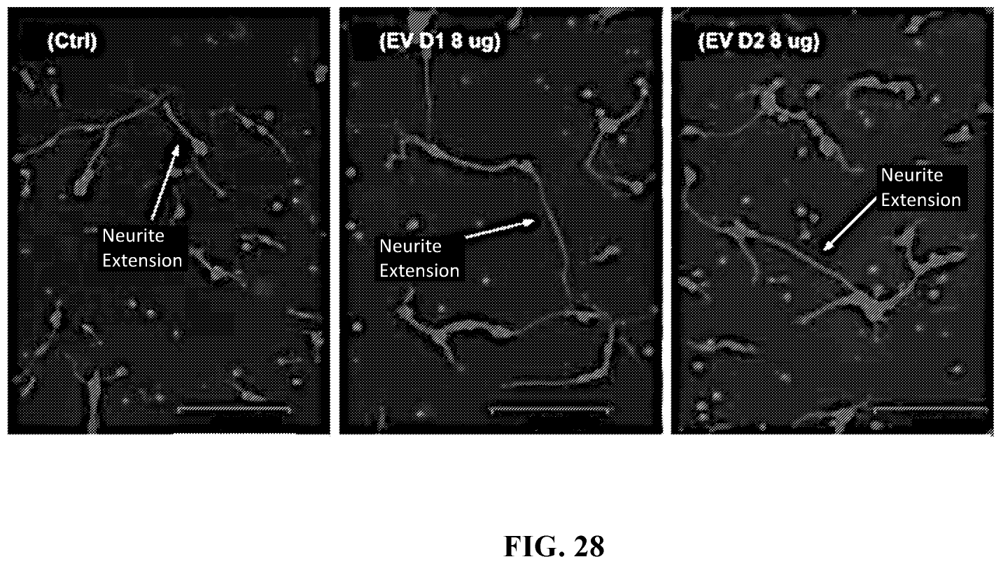

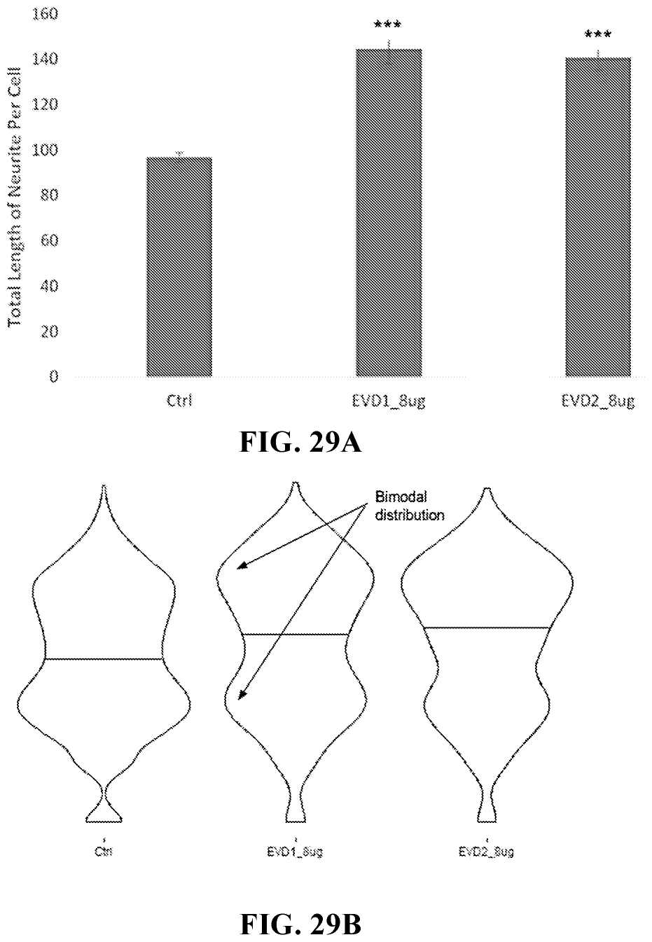

FIG. 28 shows immunostained images of single concentration (8 μg) comparing EVD1 and EVD2 with Ctrl. Neurites are traced in red (as represented in greyscale) and cell bodies are stained with DAPI (blue, as represented as greyscale). Differences (arrows pointing out neurite extensions) can be observed between the two EV types with control with apparent greater growth of neurite lengths. FIG. 29A-29B shows a graphical representation of growth distribution as a measure of total length of neurite per cell. At a selected concentration of 8 μg, both EVD1 and EVD2 showed significantly greater average total length of neurite compared to control (p<<<0.001); no significant differences were observed between the two EV populations (p=0.57). To better visualize the distribution of data, a violin plot (log of average total length of neurite) was made and a bimodal distribution was observed, albeit more prominent in the EV populations than the control. The line represents the mean.

### Isolation of Microvesicle-Only EVs Show Differences in Contributions to Growth

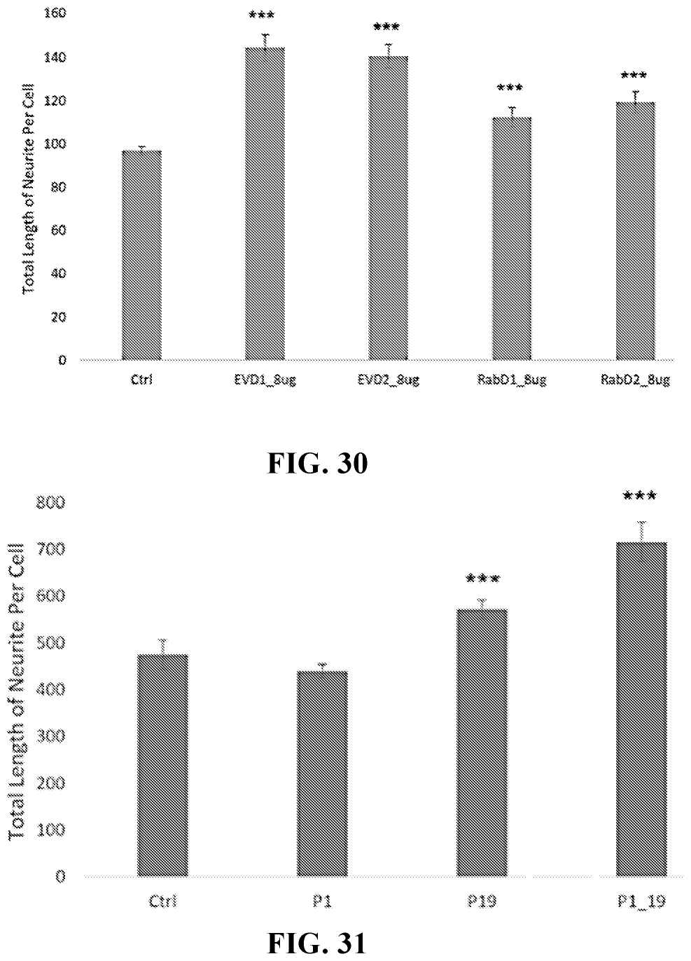

FIG. 30 shows a comparison of Rab27DKO (Exosome-Deficient EVs) with normal EVs. All EV samples (normal and Rab27DKO) were significantly different than control for average total length of neurite (p<<<0.001). EVD1 and EVD2 were also significantly different than RabD1 and RabD1 (p<<0.01). There are no significant differences between EVD1 and EVD2 (p=0.56); similarly, no significant differences were noted between RabD1 and RabD2 (p=0.26).

### Genetic Manipulation of Protocadherin Proteins in EVs Increases Growth

FIG. 31 shows the effects of electroporated Pcdh on Growth in OSNs. Note: ‘P’ was used as a shorthanded abbreviation for Pcdh and P1_19 is the combined Pcdh1 and Pcdh19. There were significant differences in average neurite length compared to control for electroporated Pcdh19 and electroporated Pcdh1+19. No statistically significant differences, however, were observed for Pcdh1 compared to control (p=0.32).

### Discussion

There was a growth-promoting role conveyed by EVs expressed in neuronal cultures. The effect seems to be standardized for EVs extracted 1-day post-culture and 2-days post-culture. This is the first-time data from OSN-EVs has ever been seen in the neuroregenerative field and indicates a clear need to further investigate the roles for neuronal-EVs in growth. Microvesicles produced part of the growth phenotype observed in vitro. Applicant successfully manipulated EV content via electroporation. A key consideration should be made for protein-protein interaction for regeneration.

### Example 7—Pcdh19 Mediates Olfactory Sensory Neuron Coalescence During Development and Regeneration

The mouse olfactory system regenerates constantly throughout life. A long-standing question is whether similar or distinct mechanisms guide olfactory sensory neurons (OSNs) during development as opposed to regeneration. A key breakthrough came with the discovery of a transient population of navigator neurons that disappear after postnatal stages. Ablation of navigator OSNs rendered later born OSNs unable to coalesce and form glomeruli, demonstrating that at least some guidance mechanisms differ between development and regeneration. To date, however, no molecules involved in OSN regeneration have been identified. Here, Applicant at least demonstrates that Pcdh19, a cell adhesion molecule and a member of the cadherin superfamily, is a mediator of OSN coalescence. Applicant compared the impact of mutating Pcdh19 on coalescence during development and during chemically-induced regeneration. At both stages, mutations in Pcdh19 led to differences in coalescence by OSNs expressing the MOR28 odorant receptor. Unexpectedly, lateral glomeruli were more affected, and males in particular showed a more severe phenotype. The impact of Pcdh19 on coalescence was significantly more pronounced during regeneration relative to development, with MOR28 OSNs displaying a much more diffuse projection pattern. To better understand why mutations in Pcdh19 differentially affected glomerular position in males and females, Applicant performed single cell analyses. Interestingly, MOR28-GFP OSNs could be subdivided into at least two major clusters. Moreover, females expressed a slightly different complement of genes from males. These features may explain the differential effects of Pcdh19 on medial and lateral glomeruli in males and females.

### Introduction

A remarkable feature of the vertebrate olfactory system is its ability to regenerate throughout life (Dorrego-Rivas & Grubb, 2022; Yu & Wu, 2017). During development, each olfactory sensory neuron (OSN) will extend an axon from the nasal epithelium into the olfactory bulb. OSNs expressing a common odorant receptor will coalesce to form medial and lateral glomeruli situated at relatively stereotyped locations within the bulb (Mombaerts et al., 1996; Ressler et al., 1994; Vassar et al., 1994). The remarkable fidelity with which this glomerular map is formed in rodents has led to intensive efforts to identify genes responsible for this process, and an increasing number of axon guidance cues have been identified that play key roles in coalescence during development (Cho et al., 2009; Lodovichi, 2021; Mori & Sakano, 2011).

The guidance mechanisms involved during development, however, appear to differ from those used during regeneration. Genetic analyses identified a critical period needed to establish the initial glomerular map in rodents (Ma et al., 2014; Tsai & Barnea, 2014; Wu et al., 2018). Neurons born prior to the ˜first week of life are essential for stabilizing the glomerular map. In the absence of these early, ‘navigator’ neurons, later born neurons can reach the approximate area where glomeruli should form. However, these OSNs will fail to coalesce, and instead project to multiple glomerular positions. As a result, navigator neurons are thought to possess distinct guidance mechanisms from later born follower neurons.

What mechanisms are used by neurons during development and regeneration, and how might they differ? In one model, later born neurons utilize adhesion or other guidance molecules as a means of following navigator neurons to their appropriate glomerular location (Ma et al., 2014; Tsai & Barnea, 2014; Wu et al., 2018). The initial set of follower neurons would presumably be used in turn during regeneration to maintain the fidelity of the map. To date however, no molecules have been identified as playing a role in OSN coalescence during regeneration, and it is unknown if any such molecule serves similar or distinct purposes during development.

To begin addressing this question, Applicant examined the role of Pcdh19, a cell adhesion molecule, for its function in OSN coalescence during development and during regeneration. Pcdh19 is one of nine members of the delta protocadherin subfamily. It was showed individual OSNs express between zero and seven family members (Bisogni et al., 2018), indicating broad expression of delta protocadherins during development. It was also demonstrated mis- and over-expression of Pcdh10 led to a transient effect on the glomerular position of OSNs expressing the SR1 (Williams et al., 2011). More recently, optogenetic stimulation demonstrated Pcdh10 expression is regulated downstream of the odorant receptor itself (Nakashima et al., 2019). Delta Pcdh17 and Pcdh20 are also known to be present within axons and glomeruli during development (Ihara et al., 2016; Lee et al., 2008).

Among the delta family members, Pcdh19 is particularly unusual in that it has been identified as the causative mutation behind a form of epilepsy known as Pcdh19-Related Epilepsy (Dibbens et al., 2008). More than 140 mutations have been identified (Kolc et al., 2019), leading it to be called the second most important gene in epilepsy. Because the disorder predominantly affects infants, Pcdh19 has been studied primarily for its role in development.

Applicant generated a mutant mouse that recapitulates a known nonsense mutation of Pcdh19 found in humans. Applicant compared the impact of this mutation on OSN coalescence during development and regeneration. During development, Applicant identified an increase in the number of glomeruli formed, consistent with a role for Pcdh19 in the terminal stages of coalescence. During regeneration, gross defects in coalescence were seen. Interestingly, these effects differ in severity between medial and lateral glomeruli, and between male and female mice. Single cell analysis provides a potential basis for explaining these differences.

### Results

Pcdh19 Expression is Grossly Maintained within the Epithelium

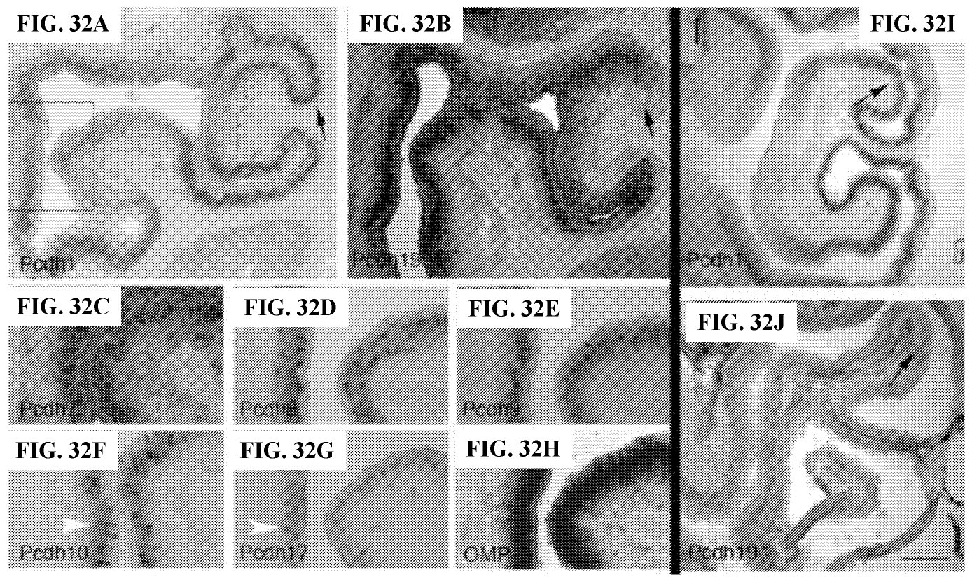

To ask if Pcdh19 may serve similar or different roles during development and regeneration, Applicant first examined its expression pattern at different time points. At embryonic day 17 (E17), Pcdh19 was expressed in a punctate pattern within the nerve layer, consistent with expression in OSNs. Interestingly, this expression pattern was not uniform, but instead was enriched in specific regions (FIG. 32B). This pattern of expression was grossly maintained through adulthood (FIG. 32B vs. FIG. 32J). Applicant tested other delta family members as well (FIG. 32C-32G), and all were expressed in a similar punctate pattern, consistent with neuronal expression (FIG. 32H). With the exception of Pcdh10 (whose expression was previously showed is modified by olfactory activity as the animal ages (Williams et al., 2011)), the remaining members were also maintained in grossly similar patterns throughout adulthood (e.g., Pcdh1, FIG. 32A vs FIG. 32I).

### Generation of Pcdh9^{E48X} Mutant Mice

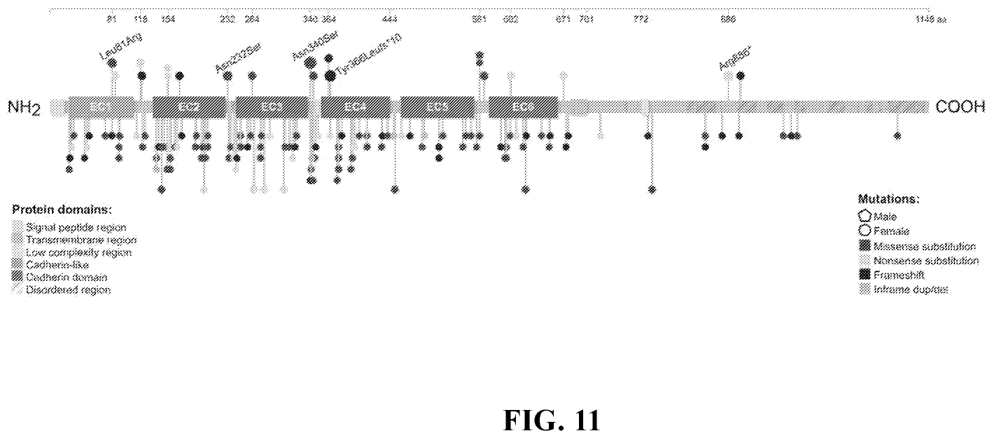

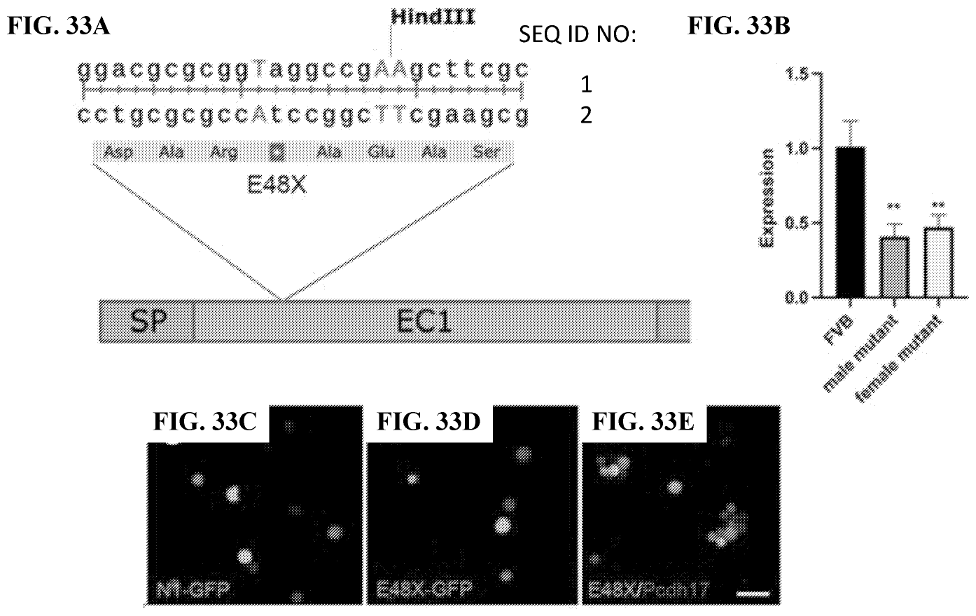

FIG. 11 shows a map of Pcdh19 CRISPR mouse mutants of known nonsense mutations. To ask how Pcdh19 influences OSN coalescence, Applicant generated a mouse mutant that recreates a known nonsense mutation (pcdh19^{E48X}) identified in humans (FIG. 33A). This mutant differs from three previously generated Pcdh19 mouse mutants, in which exon 1 (containing the entire extracellular domain) was deleted or replaced (Hayashi et al., 2017a; Hoshina et al., 2021; Pederick et al., 2016). A particularly difficult feature of studying delta protocadherins is the paucity of reliable antibodies. As a result, loss of protein expression was not confirmed in one of the three previously generated mutants, and a second used a custom antibody. Applicant tested four different commercial antibodies but were unable to find one specific to Pcdh19. Two attempts to generate Applicant's own antibody were similarly unsuccessful.

Applicant therefore assayed their mouse for anticipated effects on Pcdh19 expression and function associated with introducing a nonsense mutation. Applicant found using qRT-PCR that the mice displayed reduced overall transcription of Pcdh19, consistent with nonsense mediated decay of the mRNA (FIG. 33B). Applicant also transfected the mutant construct into K562 cells and demonstrated the pcdh19^{E48X} mutation functionally reduces Pcdh19 adhesivity (FIG. 33C-33E). As Pcdh19 is on the X chromosome, Applicant compared hemizygous mutant males and homozygous mutant females against control male samples.

### Pcdh19^{E48X} Mutants Possess Increased Glomeruli on the Lateral Surface During Development

Applicant first asked whether or not the pcdh19^{E48X} mutant affected OSN coalescence during development. Applicant crossed the mutant mouse with the MOR28-GFP marker strain, which labels OSNs expressing the MOR28 odorant receptor with GFP. Applicant examined mice for an impact on MOR28 OSN coalescence at postnatal day 14, 21, and at 6 weeks. Applicant then counted how many glomeruli were present at the medial or lateral surface. Applicant separately assessed male controls, hemizygous males, heterozygous females, and homozygous female littermates.

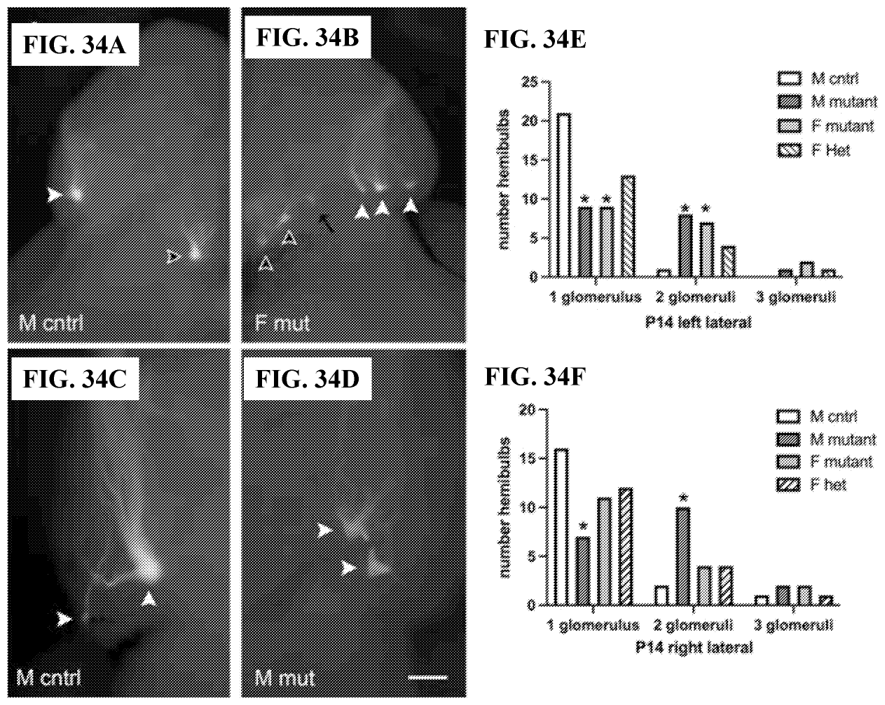

Unexpectedly, Applicant found an unusual distribution of effects that were location, age, and sex dependent. First, Applicant found that only lateral glomeruli were significantly affected (FIG. 34A). In P14 control animals, Applicant observed only one glomerulus most of the time (75-95%) at the lateral glomerular position. On rare occasions, controls displayed two lateral projections (FIG. 34C) but the second projection was noticeably smaller than the primary projection. In contrast, Applicant observed 40-50% of mutant mice (males and females) possessed two, or sometimes three, lateral glomeruli, and often these were of roughly equal size (FIGS. 34B and 34D). An intermediate phenotype was seen in heterozygous mice. Although the effect did not reach significance, the phenotype in heterozygotes mirrors that seen in mutant mice (FIG. 34E). Although Applicant also observed a trend towards an increase in glomerular number at the medial glomerulus for mutants, this effect was not statistically significant (data not shown). Applicant also tested the impact of pcdh19^{E48X} on M71-lacZ expressing OSNs at P14, P21, and six weeks. Applicant found no change in glomerular number at any stage (data not shown).

Applicant observed that this phenotype in MOR 28 expressing OSNs appeared to have a sex dependent effect. Although both male and female mice had increased numbers of left lateral glomeruli relative to male controls, only male mice showed statistically significant differences for the right lateral glomerulus (FIG. 34F).

Applicant then asked whether or not these effects on the lateral glomeruli resolved or persisted at later stages. Ectopic glomeruli formed at birth gradually disappear over time (Royal & Key, 1999). At P21, one week after the initial observations, Applicant found no differences in glomerular number among any genotype, sex, or glomerular location. Similarly, an examination of six-week-old mutant mice showed no significant differences relative to control (n=12-14 per sex and/or genotype). These results are consistent with the interpretation that Pcdh19 is important for proper targeting of MOR28 OSNs during development. OSNs are able to reach their correct location within the bulb and coalescence into glomeruli. However, differences in the number of lateral glomeruli occur in pcdh19^{E48X} mice, a phenotype that is evident particularly in males.

Pcdh19^{E48X} Mice have Defects in Coalescence During Re Generation

Applicant next examined whether Pcdh19 plays a role during regeneration to compare the effects (if any) against those seen during development. Applicant treated two to three month old mutant and control littermates with methimazole, a drug that induces widespread ablation of OSNs and subsequent replacement by stem cells (Håglin et al., 2021). Regeneration of MOR28-GFP positive OSNs was assessed at four weeks post ablation. Applicant again distinguished animals by sex and genotype, and separately assessed medial and lateral glomeruli. In control male animals, Applicant observed greater numbers of glomeruli at both surfaces, as previously reported following chemical ablation (Blanco-Hernández et al., 2012, Holbrook et al., 2014). Similarly, Applicant also saw more stray projections which had not coalesced with the main glomeruli in the target region. In mutant animals, however, this phenotype was greatly exacerbated. Applicant saw greater numbers of glomeruli of varying size, as well as a broad profusion of projections. Applicant was unable, however, to clearly resolve the patterns with standard epifluorescence.

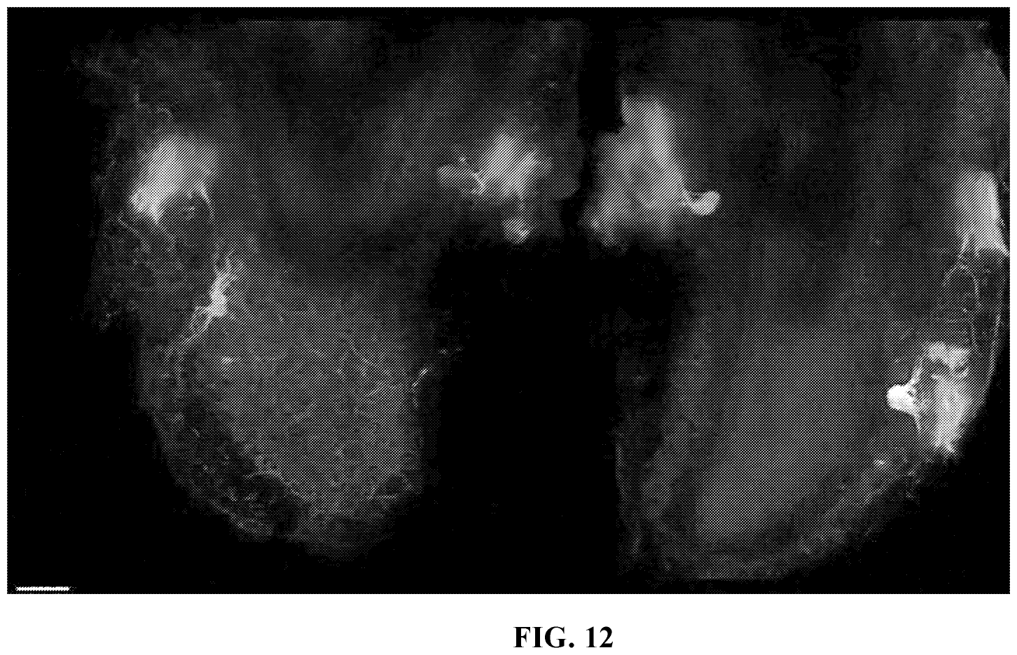

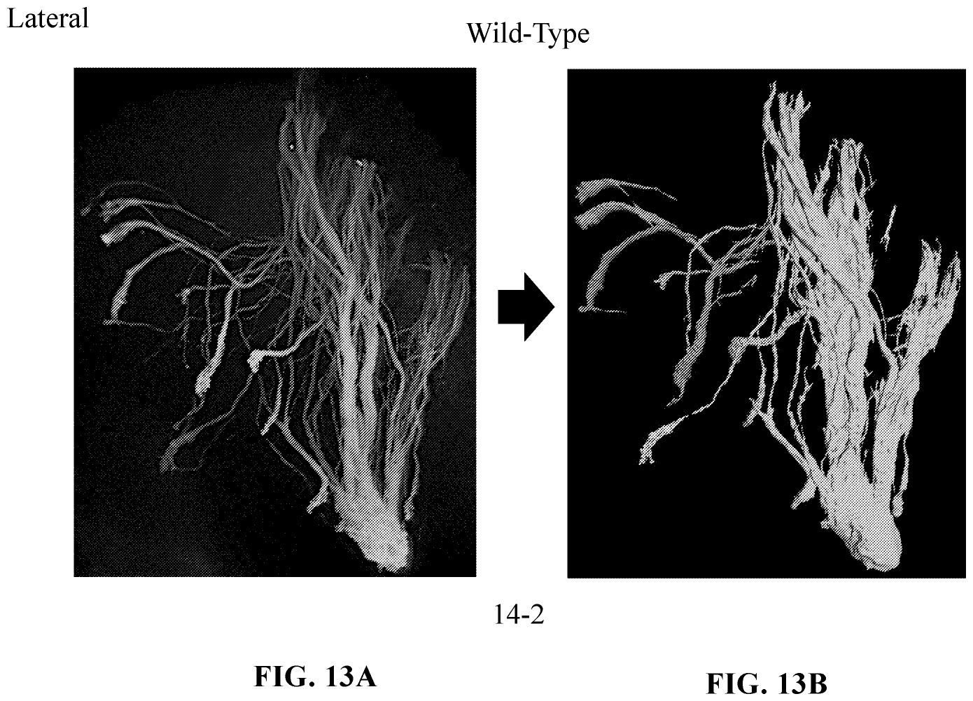

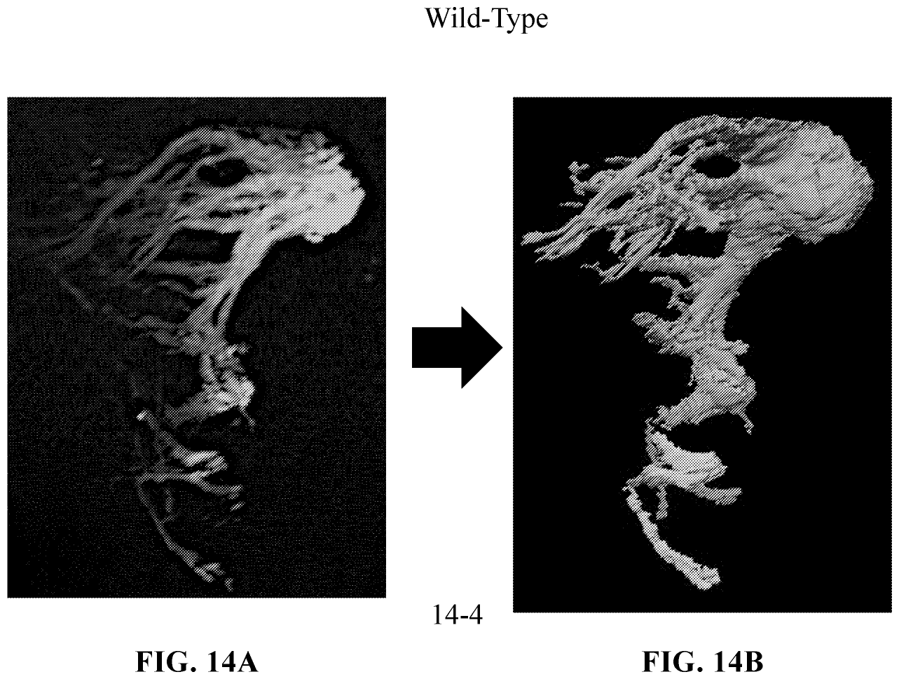

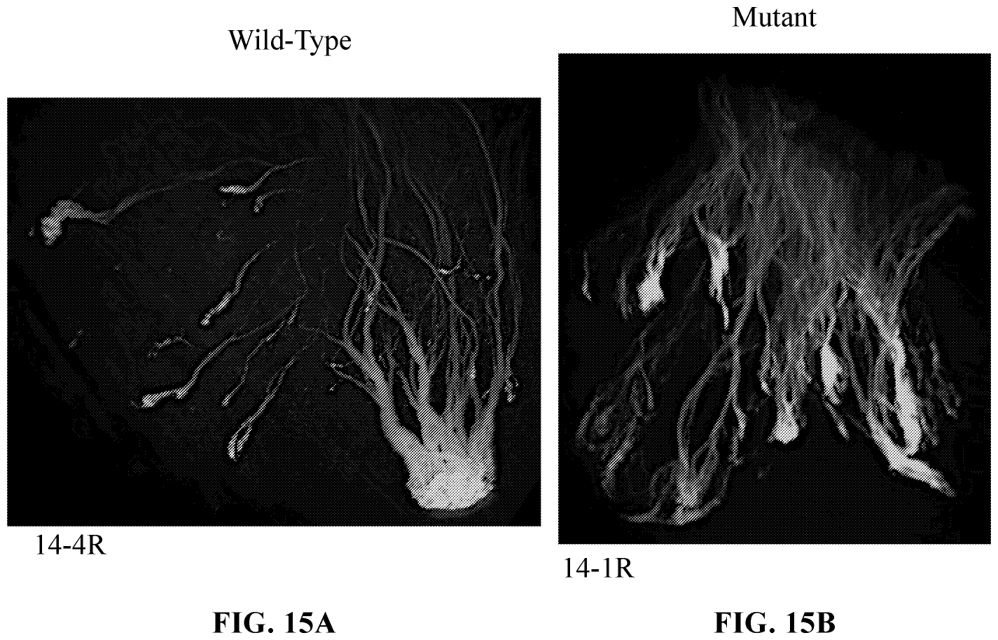

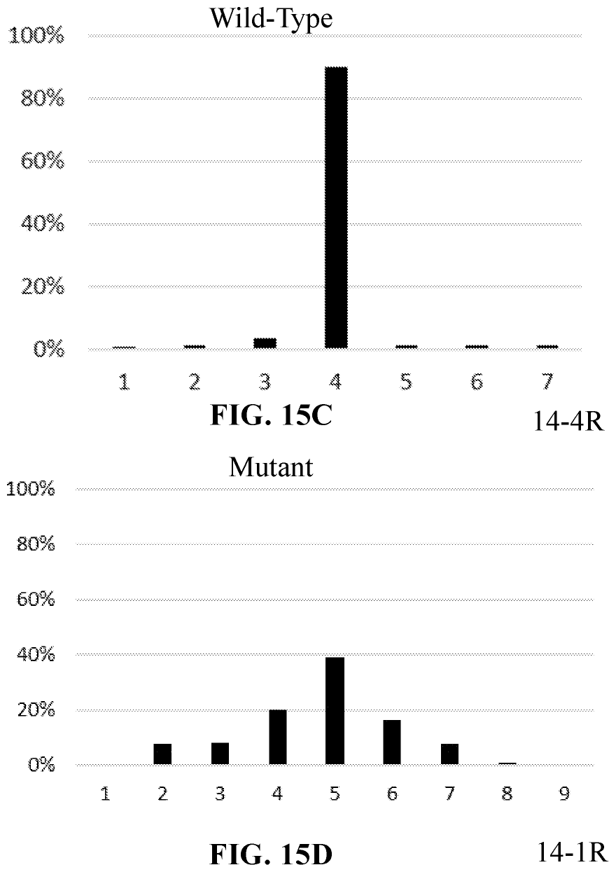

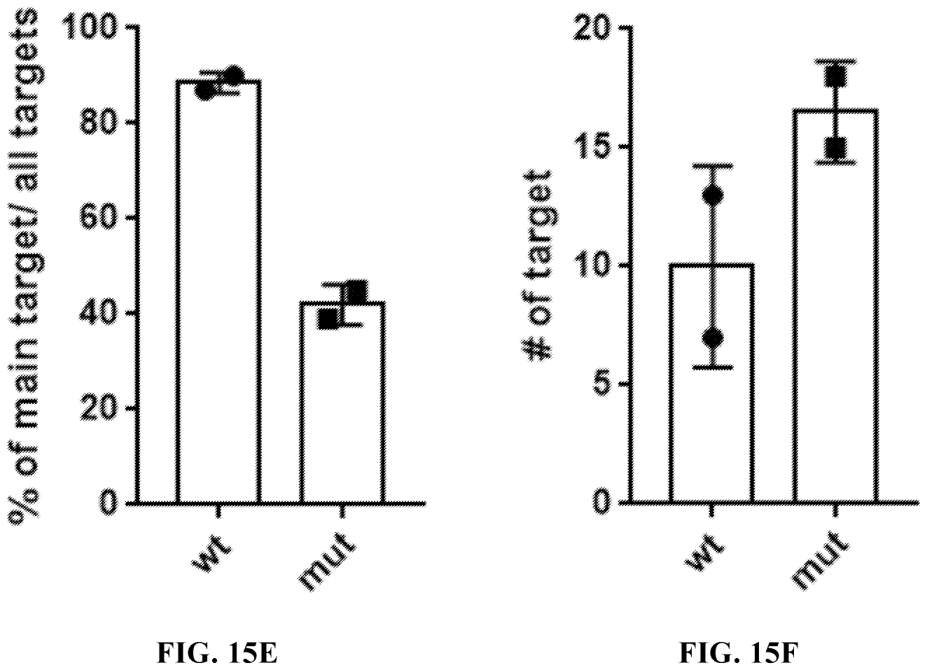

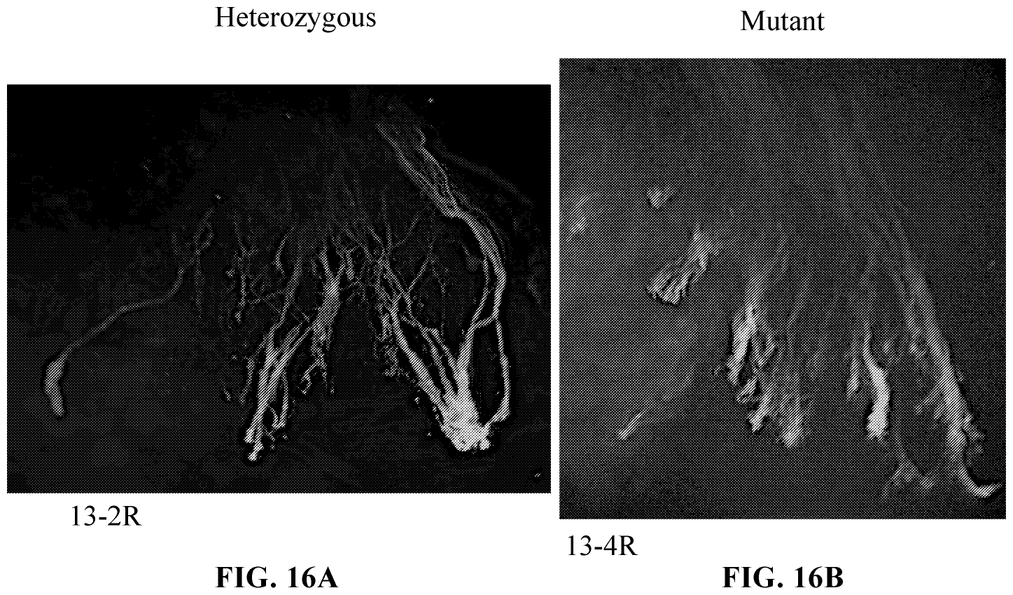

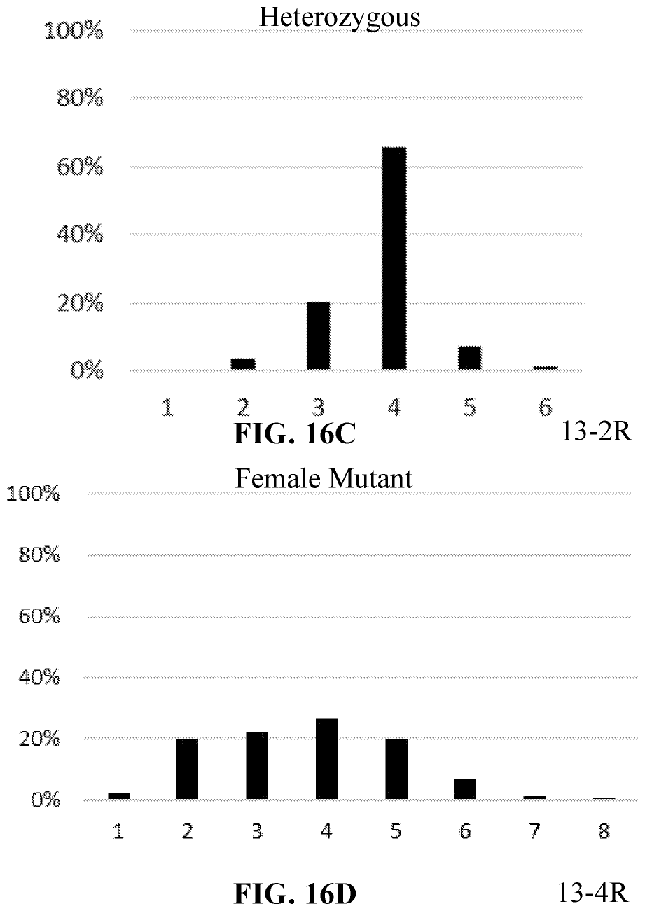

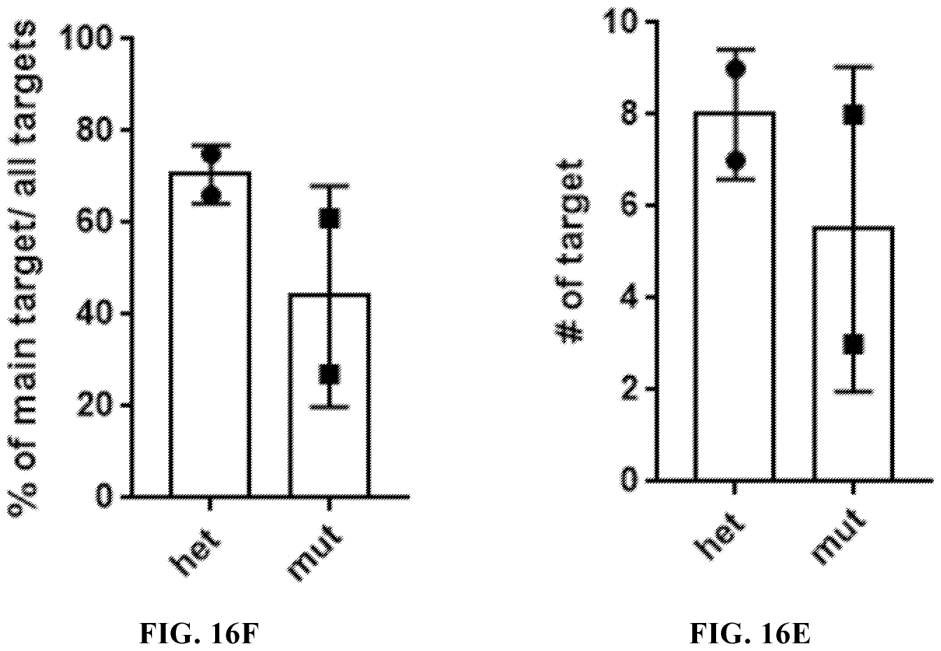

Applicant therefore utilized CUBIC-L protocols to clear the tissue and preserve GFP fluorescence (Tainaka et al., 2018), followed by light sheet microscopy in both males and female mutants and controls (see FIGS. 12, 13A-13B, 14A-14B, 15A-15F, and 16A-16F). Applicant assessed the phenotype using three different metrics. In the first, Applicant determined the spread of the signal. In the second, Applicant assessed the number of projections present. And in the third, Applicant quantified the proportion of OSNs within each projection based on GFP signal.

During development, OSNs are thought to project to a ‘neighborhood’ within the bulb, and then coalesce within this neighborhood to form glomeruli (Potter et al., 2001; Zapiec & Mombaerts, 2015). Applicant therefore asked whether or not the diffuse pattern of projections observed in mutant mice indicated a larger ‘neighborhood’ than that of control mice. Applicant performed two calculations to determine the volume of the projection pattern. Applicant first calculated the size of a three-dimensional bounding box needed to encompass the spread of projections within each animal. Applicant found no differences between mutant, heterozygous, and control mice in terms of the space taken up by the projections. Next, Applicant measured the average distance from each projection to the largest projection (based on GFP intensity). This would provide an alternative approach to assess the spread of signals surrounding the primary projection. Applicant again found no obvious differences using this metric, supporting the conclusion that the overall size of the neighborhood is unchanged in mutant animals (data not shown).

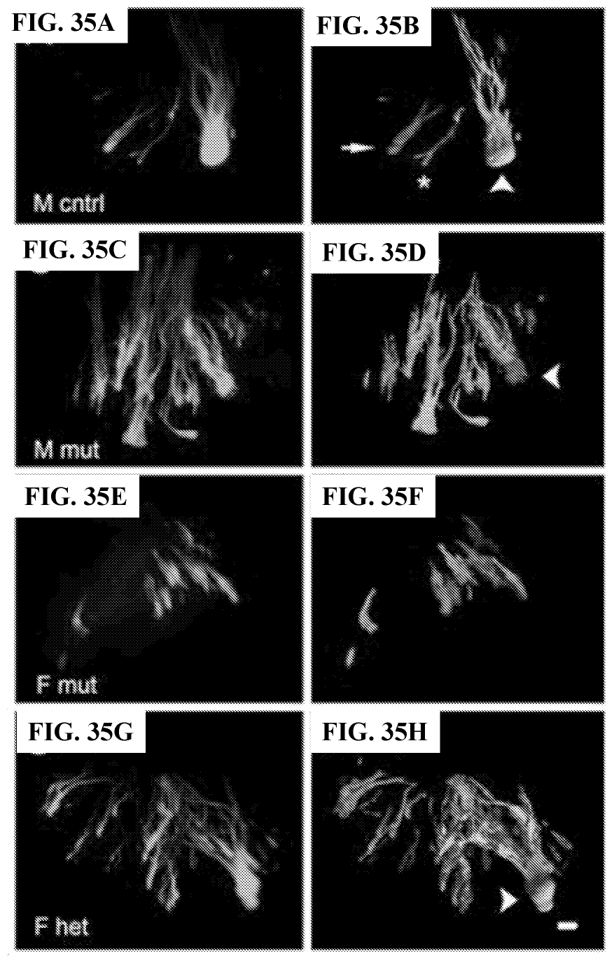

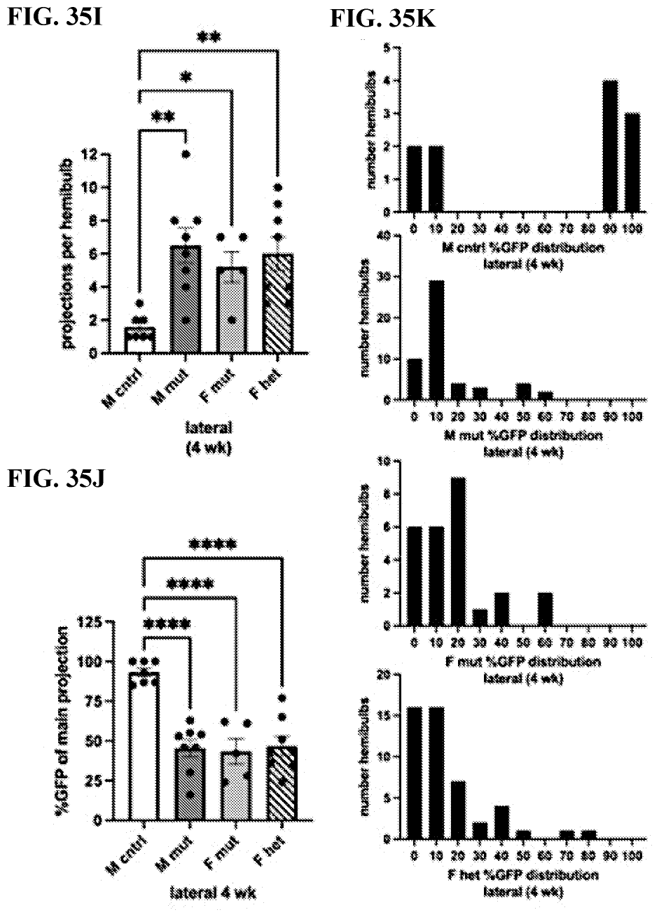

Second, Applicant asked whether or not the number of projections within the neighborhood had changed. For this analysis, Applicant counted the number of projections that comprised 3% or more of overall GFP expression within each image stack. Applicant chose 3% as a floor because weakly expressing projections were difficult to consistently detect and assess. OSNs from control mice coalesced to form approximately 1.6 projections on the lateral surface (FIG. 35I). In contrast, mutant mice projected to multiple targets. Mutant males had the most projections on average (′x=6.5) as compared to female mutants (′x=5.2) and female heterozygotes (′x=6), but all genotypes and sexes were significantly different in projection number from control males (FIG. 35I).

Third, Applicant examined the distribution of GFP within the various projections. The projection bearing the largest percentage of GFP was called the primary projection. In control mice, the majority of OSNs projected to a common glomerular location, with relatively few stray projections. Thus, on average 93% of all observed GFP was present in the main projection in control mice. In contrast, in male and female mutants, as well in heterozygous females, the proportion of GFP signal was distributed among more targets. On average, the primary projection in these mice possessed 43-47% of total GFP expression (FIG. 35J). In some cases (e.g., FIG. 35F), there was no obvious ‘primary’ projection.

The strikingly varied distribution of GFP expression between control animals and mutants is particularly evident in the histograms (FIG. 35K). In control mice, GFP signal was distributed at the extreme ends of the scale. That is, projections either contained 90%+ or 3-10% of the total GFP present, indicating most of the OSNs are contained within the primary projection. In contrast, in male mutants, most of the projections were on the far left of the spectrum, with most projections ranging between 10-50% of total GFP. This underscores the breadth and profusion of projections. Similar patterns were observed for female mutants and heterozygotes.

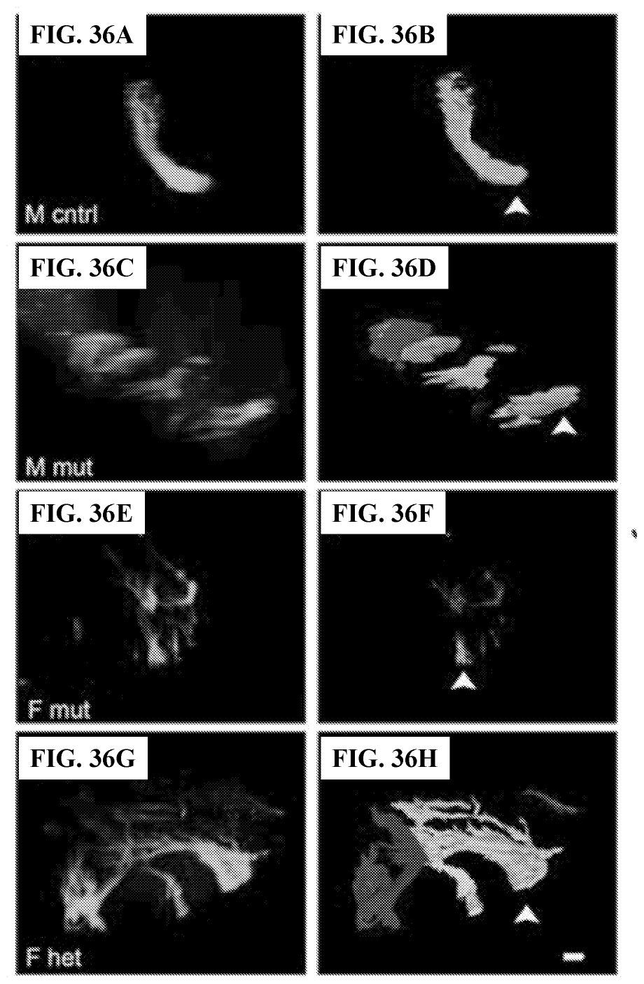

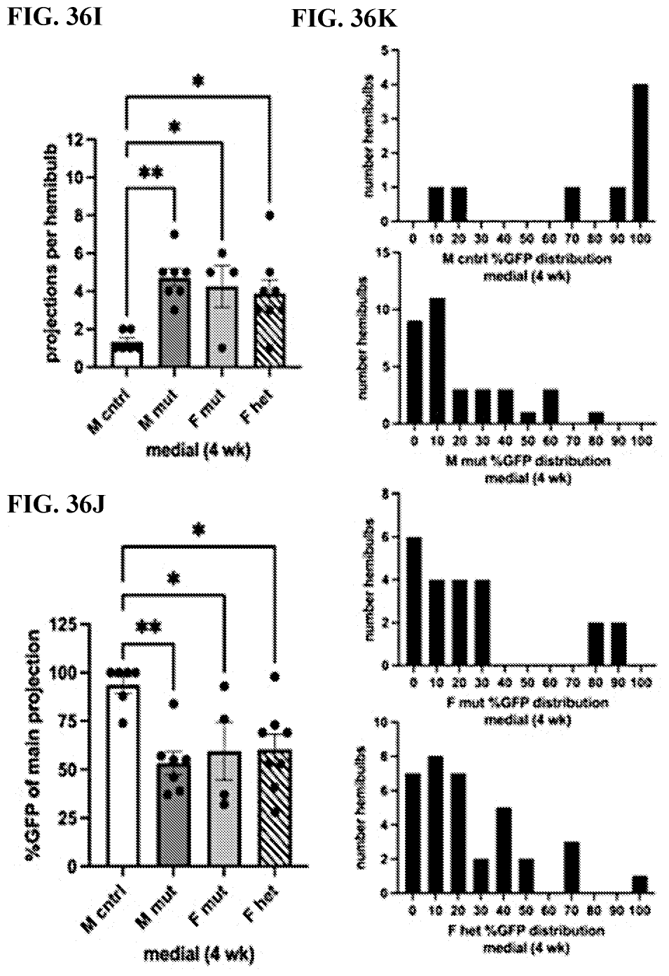

Applicant also found that, unlike during development, medial glomeruli were affected. Applicant performed the same analysis as described for the lateral glomerulus in FIG. 35A-35J. While all genotypes and sexes were affected, the degree of difference between mutants and controls on the medial surface was less than that seen for the lateral surface (FIG. 36A-36J). Interestingly, males were more affected by the pcdh19E42X mutation both in the number of projections (FIG. 36I) and in the distribution of GFP (FIG. 36J-36K).

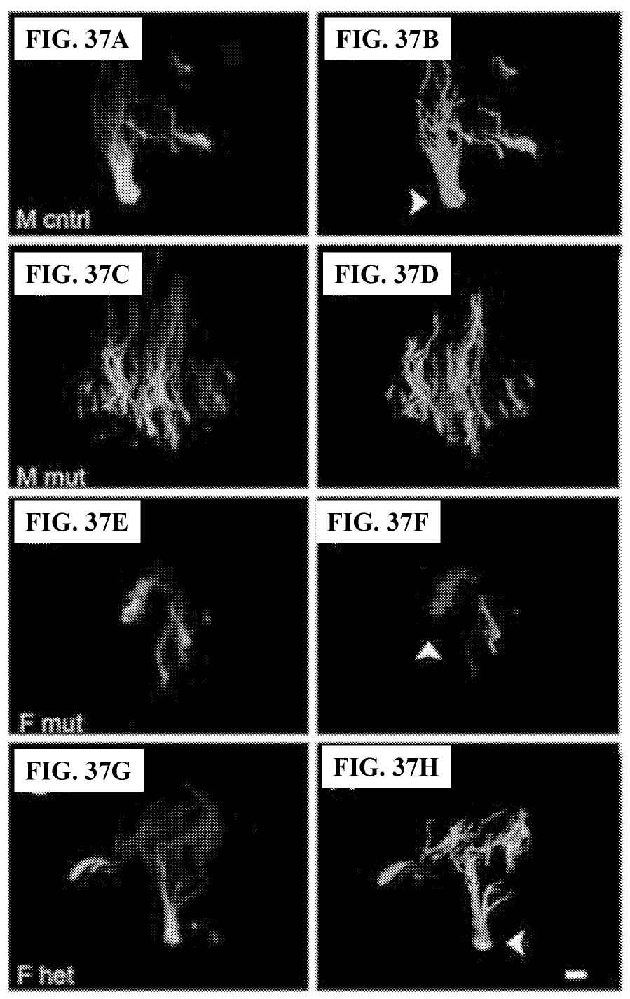

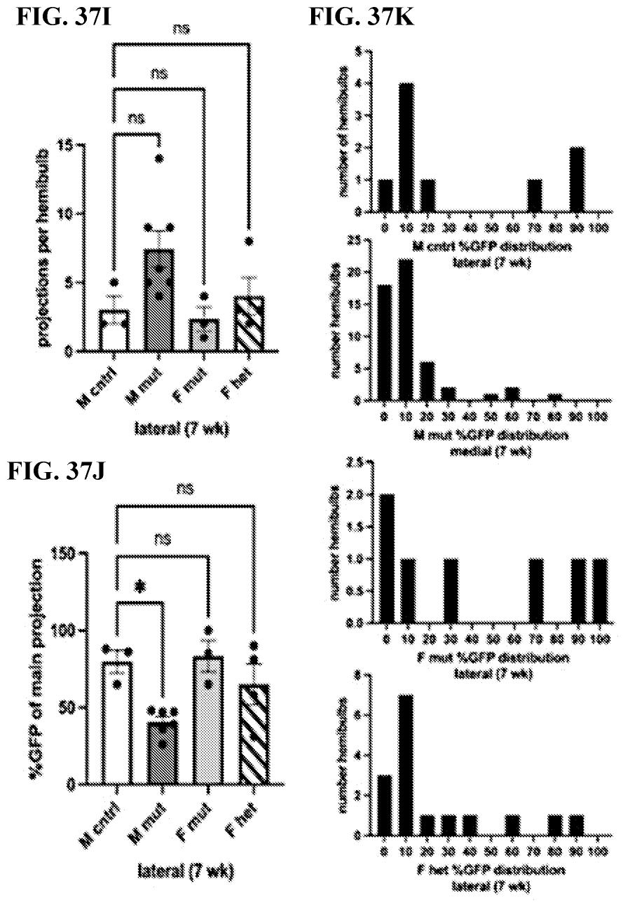

During development, the phenotype observed in lateral glomerular number at P14 was corrected by P21. Applicant therefore asked whether or not the phenotype observed at four weeks post methimazole would also recover. Applicant repeated the experiment and allowed mice to recover for seven weeks. At this time point, only the lateral glomerulus showed any effects (FIG. 37A-37J). The medial glomerulus in mutants was not significantly different from control. Furthermore, the number of projections did not differ significantly between mutants and control (FIG. 37I). Interestingly, however, the proportion of GFP present in the main projection still differed between male mutants and male controls (FIG. 37B, 37D, 37J). This is evident in the histogram distribution (FIG. 37K), which shows the GFP distribution in male mutants is still skewed towards one end of the scale.

Individual MOR28-GFP OSNs can be Subdivided Further into Two Major Clusters that Also Differ Between Males and Females

Applicant had previously shown that randomly isolated OSNs express 0-7 delta protocadherins, with no obvious pattern of expression among neurons (Bisogni et al., 2018). But given the expression pattern of delta protocadherins appear grossly similar between embryonic and adult stages (FIG. 32A-32J), Applicant asked if OSNs that express the same odorant receptor (MOR28) also express a common subset of delta family members. Applicant assayed single MOR28 OSNs isolated from male and female pups using the Nanostring platform. In addition to the delta protocadherins, Applicant included multiple representative examples of other cadherin subfamily members, including the clustered protocadherins. Applicant also included genes known to be important for OSN guidance during development.

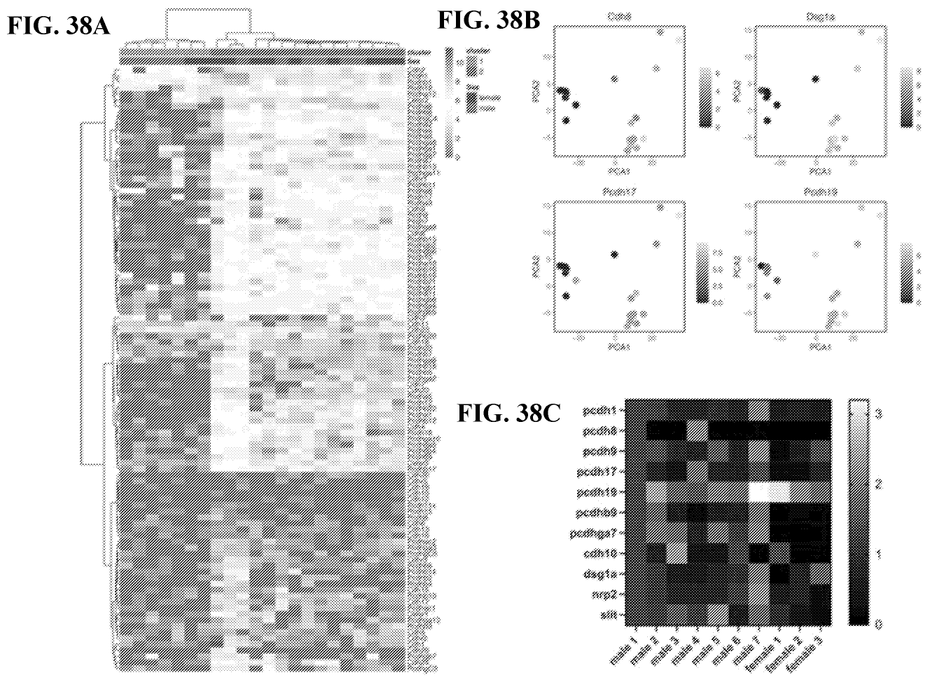

MOR28 OSNs did not express an identifiable, stereotyped pattern of delta protocadherin family members. Despite this, several unexpected features were also revealed. Strikingly, cluster analysis showed MOR28 OSNs could be subdivided into two large clusters (FIG. 38A). FIG. 38B shows an example of a subset of genes with this expression pattern. Cdh8, for example, is highly expressed in some OSNs (green dots) and weakly if at all expressed in others (purple dots). Others, such as Pcdh19, are less clearly divided into two groups, but differences in expression are still apparent within MOR28 expressing OSNs. This degree of transcriptional separation was unexpected, as all of the assayed OSNs express MOR28, and therefore were predicted a priori to be highly similar to one another. Instead, these clusters indicate a number of cadherin superfamily members and guidance genes could be used to distinguish MOR28 OSNs.

Even within a cluster, Applicant noted that the expression pattern of individual delta family members varied. While Pcdh19 was typically expressed in many MOR28 OSNs (FIG. 38B), its expression level was not constant among neurons. Thus, there appears to be smaller subdivisions within each of the two larger clusters based on expression level.

Applicant used single cell qRT-PCR employing a completely different set of primers from that used in the Nanostring analysis to validate these results (FIG. 38C). Genes identified as being differentially expressed among the two clusters (e.g., Pcdh17) were confirmed to be expressed in some MOR28 OSNs and not in others, consistent with the existence of the two clusters.

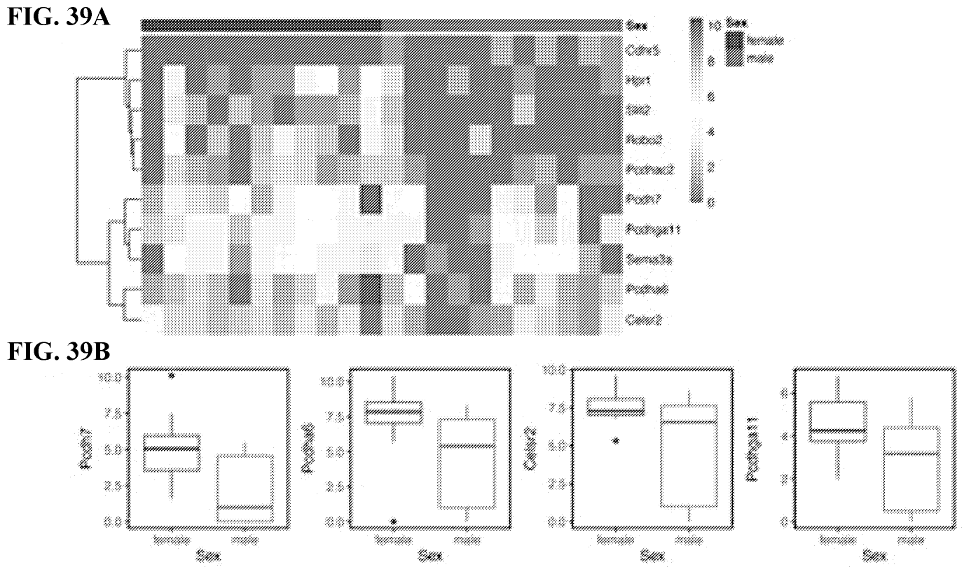

Furthermore, Applicant found males and females can also be distinguished based on the Nanostring data. Although the number of individual cells assayed was relatively small, the cluster analysis showed a subset of genes were differentially expressed in females relative to males (FIG. 39A). For example, Pcdh7 is more highly expressed in at least some female OSNs compared to male OSNs (FIG. 39B). It is important to note the variation in expression of any given gene within OSNs, as is evident from the heat map. Applicant confirmed these results using single cell qRT-PCR on male and female OSNs.

### Discussion

Here Applicant compares and contrasts phenotypes associated with mutations in Pcdh19 during development and during regeneration. During development, pcdh19^{E48X} mutants display an increase in the number of lateral glomeruli. Male mutants are particularly affected. During regeneration, MOR28 OSNs project normally to the appropriate ‘neighborhood’ within the bulb, but the number of projections is grossly increased in mutants. Moreover, the proportion of GFP distributed among these projections is different from control littermates. Both lateral and medial glomeruli are affected in male and female mutants and heterozygous females. Seven weeks after methimazole induced regeneration, however, only lateral glomeruli in male mutants are affected. Finally, single cell analysis indicates that MOR28 OSNs can be subdivided into two large clusters, and MOR28 OSNs in females possess a slightly different complement of cadherins and other genes than males.

### Similarities and Differences in Pcdh19 Function During Development and Regeneration

Given the grossly similar expression pattern for Pcdh19 between embryonic and adult stages (FIG. 32A-32J), it appeared a priori that Pcdh19 may serve a relatively constant function in the epithelium. During both development and methimazole-induced regeneration, Pcdh19 has an impact on OSN coalescence (FIGS. 34A-34F, 35A-35J, 36A-36J, and 37A-37J). Lateral glomeruli appear more affected at both stages, and males, unexpectedly, were also more affected (FIGS. 34A-34F and 37A-37J).

During regeneration, however, the impact of the pcdh19^{E48X} mutation was significantly more pronounced (FIGS. 35A-35J and 36A-36J). Unlike during development, Applicant found defects in heterozygous females, mutant females, and hemizygous males. Also unlike during development, Applicant showed an effect on both medial and lateral glomeruli at four weeks post methimazole ablation. The more widespread nature of these results underscore prior studies showing different mechanisms guide later born OSNs to their glomerular position. It's unclear, however, whether Pcdh19 function has been co-opted during regeneration to serve a new and more essential role. Alternatively, other guidance cues used during development may no longer be expressed or are co-opted, revealing an underlying, broader but constant role for Pcdh19 in coalescence. It is interesting to note that the other delta family members (with the exception of Pcdh10

(Williams et al., 2011)) also appear grossly similar in expression from embryonic to adult stages (FIG. 32A-32J). Mutant analysis may reveal phenotypes similar to that seen with Pcdh19.

The Pcdh19^{E48X} Mutant Phenotype During Development is Consistent with that Seen with Other Adhesion Molecules

The phenotype seen in pcdh19^{E48X} mutants during development is highly reminiscent of that seen with other adhesion molecules. Adhesion molecules have long been studied for their potential role in OSN coalescence (Miller et al., 2010). During development, adhesion molecules (including BIG-2, Kirrel2/3 and the clustered protocadherins) have all been demonstrated to be important for coalescence and OSN sorting (Hasegawa et al., 2008; Kaneko-Goto et al., 2008; Mountoufaris et al., 2017; Serizawa et al., 2006; Vaddadi et al., 2019).

Other cadherin superfamily members have also been examined for their expression patterns during OSN coalescence (Akins et al., 2007; Akins & Greer, 2006). Finally, delta family member Pcdh10 is known to be regulated by the odorant receptor (Nakashima et al., 2019), and can affect glomerular position when mis- and over-expressed (Williams et al., 2011).

Mutations in BIG-2 (cntn4) led to an increase in the number of ectopic glomeruli formed by MOR28 OSNs (Kaneko-Goto et al., 2008). The number of ectopic glomeruli seen with BIG-2 mutants is comparable in scale to that seen with the pcdh19^{E48X} mutant (1.8 vs 1.6 glomeruli/hemibulb, respectively). Also noteworthy is the observation that BIG-2 expression, like Pcdh19 (FIG. 38A-38C), is not uniform within MOR28 OSNs.

Similarly, deleting Kirrel2 led to an increase in ectopic glomeruli during development. The number of glomeruli increased from two to ˜three glomeruli per bulb, on par with the phenotype observed here with Pcdh19 (Vaddadi et al., 2019). In contrast to BIG-2 and Pcdh19, the expression level of Kirrel 2/3 is relatively uniform within OSNs expressing the same odorant receptor (Serizawa et al., 2006). No mention was made in the BIG-2 or Kirrel2/3 studies as to whether medial or lateral glomeruli may have been more affected.

Deleting the entire Pcdh-alpha subfamily (comprising 14 different family members) led to an increase (˜5 fold) in the number of glomeruli formed at the lateral aspect by M72 positive OSNs (Hasegawa et al., 2008). A lesser effect (˜3 fold) was observed for medial glomeruli, paralleling the results showing a more significant impact on lateral glomeruli in pcdh19^{E48X} mutants. When all clustered protocadherins (comprising 58 members in mice) were deleted, OSNs broadly failed to coalesce (Mountoufaris et al., 2017). No indication was given as to whether or not males or females were especially affected by loss of the clustered protocadherins.

Applicant also note that other mutations in Pcdh19 have been previously generated (Hayashi et al., 2017b; Hoshina et al., 2021; Pederick et al., 2016). These mutants showed subtle defects in cortical neuron composition (Galindo-Riera et al., 2021), but no obvious changes in axon tract formation in the brain. The effects Applicant saw during development on OSN coalescence may have been possible because of the exquisite resolution afforded by the MOR28-GFP marker. On the other hand, it is also possible that delta family members serve different purposes in different systems.

Finally, it is important to note that mutations in Pcdh19 are unlikely to affect all OSN populations. Given the large number of guidance cues and cadherin superfamily members expressed by MOR28 OSNs (FIGS. 38A-38C and 39A-39B), the punctate pattern of expression (FIG. 32A-32J), and the prior results indicating randomly selected OSNs express between 0-7 delta family members (Bisogni et al., 2018), OSNs expressing different receptors likely will all display different effects as a result of mutating Pcdh19. Consistent with this, Applicant found no effect on glomerular number by OSNs expressing M71 in the presence of the pcdh19^{E48X} mutation (data not shown).

Pcdh19^{E48X} Affects Lateral Glomeruli More than Medial Glomeruli During Development and Regeneration.

While numerous adhesion molecules and guidance cues have been identified for their role in OSN coalescence during development, Pcdh19 is the first molecule, adhesion or otherwise, to be examined for its function during OSN regeneration. The impact on coalescence during methimazole-induced regeneration was noticeably more dramatic than that observed during development (FIGS. 35A-35J, 36A-36J, and 37A-37J). Applicant found a greater impact on lateral glomeruli than on medial glomeruli. While both medial and lateral glomeruli were affected at four weeks post methimazole, only lateral glomeruli were still affected after seven weeks. Why are lateral glomeruli more strongly affected than medial glomeruli? Applicant postulated the variable expression of Pcdh19 within MOR28 OSNs contributes to this effect. Just as BIG-2 is variably expressed among MOR28 OSNs during development, the mosaic expression of Pcdh19 can potentially lead to a differential impact on OSNs that target the medial or lateral surface. This is consistent with morphological examination of P2-expressing OSNs showing differential numbers of glomeruli forming at the medial and lateral surfaces during development (Royal & Key, 1999). While IGF2 appears to globally influence medial or lateral OSN projection (Scolnick et al., 2008), data from the pcdh19^{E48X} mutant, as well as that of the alpha protocadherins (Hasegawa et al., 2008), indicates the presence of further fine-tuning mechanisms within OSNs as they approach their terminal position within the medial and lateral surface.

The single cell Nanostring data suggests that there may be a larger number of cues yet to be identified which regulate medial or lateral glomeruli coalescence. While Applicant initially speculated MOR28 OSNs express a stereotyped pattern of delta protocadherin family members, Applicant did not find this to be the case. Instead, Applicant discovered MOR28 OSNs could be subdivided into two large clusters based on the expression of other cadherin superfamily members and other genes. Applicant further showed using single cell qRT-PCR that genes were expressed in patterns that reflects the cluster analysis. That is, some genes were highly expressed in some MOR28 OSNs and lowly expressed or absent in others.

It is tempting to speculate that these two clusters reflect MOR28 OSNs that will project to the medial or lateral surface. If this is the case, it would indicate distinct complements of genes interact with one another to establish the olfactory map on either aspect of the bulb. An important caveat is that this single cell analysis was performed at early stages of postnatal development. It is therefore not clear how this complement may change over time. Other single studies comparing navigator and older OSNs also did not clearly identify factors that could mediate differential guidance at differing stages, suggesting the need for more targeted or higher resolution assays (Wu et al., 2018).

Pcdh19^{E48X} Appears to Affect Males More than Females During Development and Regeneration.

Perhaps surprisingly, Applicant also found differences in the impact of pcdh19^{E48X} mutant on coalescence in males and females. Males were more strongly affected both during development and during regeneration (FIGS. 3, 5 and 6). Sex in general has long been considered an important component of olfaction, and human females have consistently been found to possess better olfactory capabilities than males (Sorokowski et al., 2019).

While the basis for sex differences in olfactory acuity are still under study, the single cell studies suggest a potential rationale for the sex specific differences Applicant observed during regeneration. Although the number of individual cells used for the Nanostring analysis is relatively small, Applicant found subtle differences in expression of adhesion molecules between male and female mice (FIG. 39A-39A). These differences are not absolute (e.g., male vs. female) but rather female OSNs appear to be more likely to express these particular genes within MOR28 OSNs. While a great deal more work is needed to validate these initial observations, they may explain why females are more resistant to the effects of the mutation because they express additional genes that can compensate for mutations in Pcdh19. Applicant notes that a delta protocadherin family member (Pcdh20) was shown to have differences in male and female glomerular expression in adult animals (Lee et al., 2008), consistent with the Nanostring results.

In conclusion, Applicant have identified Pcdh19 as a mediator of OSN coalescence during development and regeneration. Applicant uncovered differential effects on medial and lateral glomeruli, and on male and female mutant mice.

### Methods

### In Situ Hybridization

This was performed as previously described (Williams et al., 2007).

### Animal Care and Handling

All animal protocols were reviewed and approved by Cornell's Institutional Animal Care and Use Committee. Pcdh19^{E48X} mice were outbred six generations to FVB mice to reduce potential off-target effects. MOR28-GFP mice were a generous gift of Gilad Barnea, and are maintained in a mixed B6:129 background. Female mutants were crossed with MOR28-GFP males to generate hemizygous males and heterozygous females, and F1 animals were intercrossed. Because Pcdh19 is on the X chromosome, only male control (non-mutant) animals were obtained for comparison. Animals were maintained on a standard chow diet and fed ad libitum.

### Generation of Pcdh9^{E48X} Mice

A guide RNA was generated by creating a template comprised of primers surrounding the PAM site. mRNA was generated and sent to the Cornell Transgenic Core for injection into F1 B6/FVB mice.

### Methimazole Ablation

Two to three month old littermates were weighed and injected intraperitoneally with methimazole (100 mg/kg). Applicant found, as previously described (Håglin et al., 2021) more consistent ablation with a higher dose of methimazole. Mice were allowed to recover for four or seven weeks prior to sacrifice.

### Whole Mount GFP Visualization

Whole mount olfactory bulbs still attached to the remainder of the brain were dissected from freshly euthanized animals, and visualized with a Leica MZFLIII microscope fitted with a GFP dichroic filter. Images were captured with an Olympus DP70 camera.

### K562 Cell Transfection

K562 cells were transfected as described (Bisogni et al., 2018) and imaged on a Leica LSM 510 confocal microscope.

### Lightsheet Microscopy

Protocols for lightsheet microscopy are based on those described by (Tainaka et al., 2018) with the following modifications. Mice were anesthetized with avertin and then transcardially perfused with 20 mls of chilled phosphate buffered saline (PBS) followed by eight mls of phosphate buffered 4% paraformaldehyde (PFA) and 10 mls of PBS. Brains with attached olfactory bulbs were dissected out and fixed overnight with 4% PFA. After several washes with PBS to remove fixative, brains and bulbs were placed in 50% CUBIC-L at 37 degrees C. overnight. Samples were changed to 100% CUBIC-L after 24 hours and left at 37 degrees for an additional four days. CUBIC-L solution was changed at days one and three during the four day incubation. After washing with PBS, samples were placed in 50% CUBIC-RA for 24 hours at room temperature, and then 100% CUBIC-RA for at least 24 hours prior to embedding. Brains were bisected laterally and half was embedded in CUBIC-RA containing 1.6% agarose in PMMA cuvettes. Samples were imaged with the LaVision Bio Tec Lightsheet Ultramicroscope II. Bulbar structure was illuminated using the 640 nm laser, and GFP signal visualized at 480 nm. All images were taken at the same magnification with a Z-axis step size of 10 um. Because samples had one or two GFP markers present, laser power for each channel was adjusted accordingly for each sample to maximize intensity distribution.

### Lightsheet Image Analysis

Images were uploaded to Arivis 4D 3.5. Using the GFP signal detected by the 488-laser, an intensity threshold was set as determined by the intensity of the GFP signal to be able to segment the projections. Thresholds ranged from 5000 to 7000 for each medial and lateral sample. Segmentation was confirmed in 2D prior to object identification in 3D. Once projections were segmented, the sum-intensity of each projection was calculated based on GFP signal intensity. Objects that were inaccurately segmented together were split using an automated filter set at 50% sensitivity.

### Statistical Analysis

Prism (9.0) was used for all statistical analysis.

### Single Cell Isolation and Nanostring Analysis

Applicant followed established protocols (Bisogni et al., 2018) to isolate individual OSNs from P7-P9 male or female pups from MOR28 GFP. After visually separating the two sexes, epithelia were dissected out in 1× Modified Eagles Medium with Temin's modification. The remainder of the protocol followed previously published procedures.

### Nanostring Analysis

### REFERENCES RELATED TO EXAMPLE 7

Akins, M. R., Benson, D. L., & Greer, C. A. (2007). Cadherin expression in the developing mouse olfactory system. The Journal of Comparative Neurology, 501(4), 483-497. https://doi.org/10.1002/cne.21270
Akins, M. R., & Greer, C. A. (2006). Axon behavior in the olfactory nerve reflects the involvement of catenin-cadherin mediated adhesion. The Journal of Comparative Neurology, 499(6), 979-989. https://doi.org/10.1002/cne.21147
Bisogni, A. J., Ghazanfar, S., Williams, E. O., Marsh, H. M., Yang, J. Y., & Lin, D. M. (2018). Tuning of delta-protocadherin adhesion through combinatorial diversity. ELife, 7, 400.
Blanco-Hernández, E., Valle-Leija, P., Zomosa-Signoret, V., Drucker-Colín, R., & Vidaltamayo, R. (2012). Odor memory stability after reinnervation of the olfactory bulb. PloS One, 7(10), e46338.
Cho, J. H., Prince, J. E. A., & Cloutier, J.-F. (2009). Axon guidance events in the wiring of the mammalian olfactory system. Molecular Neurobiology, 39(1), 1-9. https://doi.org/10.1007/s12035-008-8047-7
Dibbens, L. M., Tarpey, P. S., Hynes, K., Bayly, M. A., Scheffer, I. E., Smith, R., Bomar, J., Sutton, E., Vandeleur, L., Shoubridge, C., Edkins, S., Turner, S. J., Stevens, C., O'Meara, S., Tofts, C., Barthorpe, S., Buck, G., Cole, J., Halliday, K., . . . Gécz, J. (2008). X-linked protocadherin 19 mutations cause female-limited epilepsy and cognitive impairment. Nature Genetics, 40(6), 776-781.
Dorrego-Rivas, A., & Grubb, M. S. (2022). Developing and maintaining a nose-to-brain map of odorant identity. Open Biology, 12(6), 220053. https://doi.org/10.1098/rsob.220053
Galindo-Riera, N., Newbold, S. A., Sledziowska, M., Llinares-Benadero, C., Griffiths, J., Mire, E., & Martinez-Garay, I. (2021). Cellular and Behavioral Characterization of Pcdh19 Mutant Mice: Subtle Molecular Changes, Increased Exploratory Behavior and an Impact of Social Environment. ENeuro, 8(4), ENEURO.0510-20.2021. https://doi.org/10.1523/ENEURO.0510-20.2021.
Håglin, S., Bohm, S., & Berghard, A. (2021). Single or Repeated Ablation of Mouse Olfactory Epithelium by Methimazole. Bio-Protocol, 11(8), e3983. https://doi.org/10.21769/BioProtoc.3983
Hasegawa, S., Hamada, S., Kumode, Y., Esumi, S., Katori, S., Fukuda, E., Uchiyama, Y., Hirabayashi, T., Mombaerts, P., & Yagi, T. (2008). The protocadherin-alpha family is involved in axonal coalescence of olfactory sensory neurons into glomeruli of the olfactory bulb in mouse. Molecular and Cellular Neurosciences, 38(1), 66-79.
Hayashi, S., Inoue, Y., Hattori, S., Kaneko, M., Shioi, G., Miyakawa, T., & Takeichi, M. (2017a). Loss of X-linked Protocadherin-19 differentially affects the behavior of heterozygous female and hemizygous male mice. Scientific Reports, 7(1), 5801-5815.
Hayashi, S., Inoue, Y., Hattori, S., Kaneko, M., Shioi, G., Miyakawa, T., & Takeichi, M. (2017b). Loss of X-linked Protocadherin-19 differentially affects the behavior of heterozygous female and hemizygous male mice. Scientific Reports, 7(1), 5801-5815.
Holbrook, E. H., Iwema, C. L., Peluso, C. E., & Schwob, J. E. (2014). The Regeneration of P2 Olfactory Sensory Neurons Is Selectively Impaired Following Methyl Bromide Lesion. Chemical Senses, 39(7), 601-616. https://doi.org/10.1093/chemse/bju033
Hoshina, N., Johnson-Venkatesh, E. M., Hoshina, M., & Umemori, H. (2021). Female-specific synaptic dysfunction and cognitive impairment in a mouse model of PCDH19 disorder. Science (New York, N.Y.), 372(6539), eaaz3893. https://doi.org/10.1126/science.aaz3893 Ihara, N., Nakashima, A., Hoshina, N., Ikegaya, Y., & Takeuchi, H. (2016). Differential expression of axon-sorting molecules in\textasciitildemouse olfactory sensory neurons. The European Journal of Neuroscience, 44(3), 1998-2003.
Kaneko-Goto, T., Yoshihara, S.-I., Miyazaki, H., & Yoshihara, Y. (2008). BIG-2 mediates olfactory axon convergence to target glomeruli. Neuron, 57(6), 834-846. https://doi.org/10.1016/j.neuron.2008.01.023
Kolc, K. L., Sadleir, L. G., Scheffer, I. E., Ivancevic, A., Roberts, R., Pham, D. H., & Gécz, J. (2019). A systematic review and meta-analysis of 271 PCDH19-variant individuals identifies psychiatric comorbidities, and association of seizure onset and disease severity. Molecular Psychiatry, 24(2), 241-251.
Lee, W., Cheng, T.-W., & Gong, Q. (2008). Olfactory sensory neuron-specific and sexually dimorphic expression of protocadherin 20. The Journal of Comparative Neurology, 507(1), 1076-1086. https://doi.org/10.1002/cne.21569.
Lodovichi, C. (2021). Topographic organization in the olfactory bulb. Cell and Tissue Research, 383(1), 457-472. https://doi.org/10.1007/s00441-020-03348-w.
Ma, L., Wu, Y., Qiu, Q., Scheerer, H., Moran, A., & Yu, C. R. (2014). A developmental switch of axon targeting in the continuously regenerating mouse olfactory system. Science (New York, N.Y.), 344(6180), 194-197.
Miller, A. M., Maurer, L. R., Zou, D.-J., Firestein, S., & Greer, C. A. (2010). Axon fasciculation in the developing olfactory nerve. Neural Development, 5, 20. https://doi.org/10.1186/1749-8104-5-20.
Mombaerts, P., Wang, F., Dulac, C., Chao, S. K., Nemes, A., Mendelsohn, M., Edmondson, J., & Axel, R. (1996). Visualizing an olfactory sensory map. Cell, 87(4), 675-686.
Mori, K., & Sakano, H. (2011). How is the olfactory map formed and interpreted in the mammalian brain? Annual Review of Neuroscience, 34(1), 467-499.
Mountoufaris, G., Chen, W. V., Hirabayashi, Y., O'Keeffe, S., Chevee, M., Nwakeze, C. L., Polleux, F., & Maniatis, T. (2017). Multicluster Pcdh diversity is required for mouse olfactory neural circuit assembly. Science (New York, N.Y.), 356(6336), 411-414.
Nakashima, A., Ihara, N., Shigeta, M., Kiyonari, H., Ikegaya, Y., & Takeuchi, H. (2019). Structured spike series specify gene expression patterns for olfactory circuit formation. Science (New York, N.Y.), 365(6448), eaaw5030. https://doi.org/10.1126/science.aaw5030.
Pederick, D. T., Homan, C. C., Jaehne, E. J., Piltz, S. G., Haines, B. P., Baune, B. T., Jolly, L. A., Hughes, J. N., Gécz, J., & Thomas, P. Q. (2016). Pcdh19 Loss-of-Function Increases Neuronal Migration In Vitro but is Dispensable for Brain Development in Mice. Scientific Reports, 1-10.
Potter, S. M., Zheng, C., Koos, D. S., Feinstein, P., Fraser, S. E., & Mombaerts, P. (2001). Structure and emergence of specific olfactory glomeruli in the mouse. The Journal of Neuroscience: The Official Journal of the Society for Neuroscience, 21(24), 9713-9723.
Ressler, K. J., Sullivan, S. L., & Buck, L. B. (1994). Information coding in the olfactory system: Evidence for a stereotyped and highly organized epitope map in the olfactory bulb. Cell, 79(7), 1245-1255.
Royal, S. J., & Key, B. (1999). Development of P2 olfactory glomeruli in P2-internal ribosome entry site-tau-LacZ transgenic mice. The Journal of Neuroscience: The Official Journal of the Society for Neuroscience, 19(22), 9856-9864.
Scolnick, J. A., Cui, K., Duggan, C. D., Xuan, S., Yuan, X.-B., Efstratiadis, A., & Ngai, J. (2008). Role of IGF signaling in olfactory sensory map formation and axon guidance. Neuron, 57(6), 847-857. https://doi.org/10.1016/j.neuron.2008.01.027.
Serizawa, S., Miyamichi, K., Takeuchi, H., Yamagishi, Y., Suzuki, M., & Sakano, H. (2006). A neuronal identity code for the odorant receptor-specific and activity-dependent axon sorting. Cell, 127(5), 1057-1069.
Sorokowski, P., Karwowski, M., Misiak, M., Marczak, M. K., Dziekan, M., Hummel, T., & Sorokowska, A. (2019). Sex Differences in Human Olfaction: A Meta-Analysis. Frontiers in Psychology, 10. https://www.frontiersin.org/articles/10.3389/fpsyg.2019.00242.
Tainaka, K., Murakami, T. C., Susaki, E. A., Shimizu, C., Saito, R., Takahashi, K., Hayashi-Takagi, A., Sekiya, H., Arima, Y., Nojima, S., Ikemura, M., Ushiku, T., Shimizu, Y., Murakami, M., Tanaka, K. F., Iino, M., Kasai, H., Sasaoka, T., Kobayashi, K., . . . Ueda, H. R. (2018). Chemical Landscape for Tissue Clearing Based on Hydrophilic Reagents. Cell Reports, 24(8), 2196-2210.e9.
Tsai, L., & Barnea, G. (2014). A critical period defined by axon-targeting mechanisms in the murine olfactory bulb. Science (New York, N.Y.), 344(6180), 197-200.
Vaddadi, N., Iversen, K., Raja, R., Phen, A., Brignall, A., Dumontier, E., & Cloutier, J.-F. (2019). Kirrel2 is differentially required in populations of olfactory sensory neurons for the targeting of axons in the olfactory bulb. Development (Cambridge, England), 146(11), dev173310.
Vassar, R., Chao, S. K., Sitcheran, R., Nunez, J. M., Vosshall, L. B., & Axel, R. (1994). Topographic organization of sensory projections to the olfactory bulb. Cell, 79(6), 981-991.
Walz, A., Mombaerts, P., Greer, C. A., & Treloar, H. B. (2006). Disrupted compartmental organization of axons and dendrites within olfactory glomeruli of mice deficient in the olfactory cell adhesion molecule, OCAM. Molecular and Cellular Neurosciences, 32(1-2), 1-14. https://doi.org/10.1016/j.mcn.2006.01.013.
Williams, E. O., Sickles, H. M., Dooley, A. L., Palumbos, S., Bisogni, A. J., & Lin, D. M. (2011). Delta Protocadherin 10 is Regulated by Activity in the Mouse Main Olfactory System. Frontiers in Neural Circuits, 5, 9.
Williams, E. O., Xiao, Y., Sickles, H. M., Shafer, P., Yona, G., Yang, J. Y. H., & Lin, D. M. (2007). Novel subdomains of the mouse olfactory bulb defined by molecular heterogeneity in the nascent external plexiform and glomerular layers. BMC Developmental Biology, 7(1), 48.
Wu, Y., Ma, L., Duyck, K., Long, C. C., Moran, A., Scheerer, H., Blanck, J., Peak, A., Box, A., Perera, A., & Yu, C. R. (2018). A Population of Navigator Neurons Is Essential for Olfactory Map Formation during the Critical Period. Neuron, 100(5), 1066-1082.e6.
Yu, C. R., & Wu, Y. (2017). Regeneration and rewiring of rodent olfactory sensory neurons. Experimental Neurology, 287, 395-408.
Zapiec, B., & Mombaerts, P. (2015). Multiplex assessment of the positions of odorant receptor-specific glomeruli in the mouse olfactory bulb by serial two-photon tomography. Proceedings of the National Academy of Sciences of the United States of America, 112(43), E5873-82.

### Example 8—Pcdh19 Promotes Growth of Dorsal Root Ganglion Neurons In Vitro and Transected Peripheral Nerves

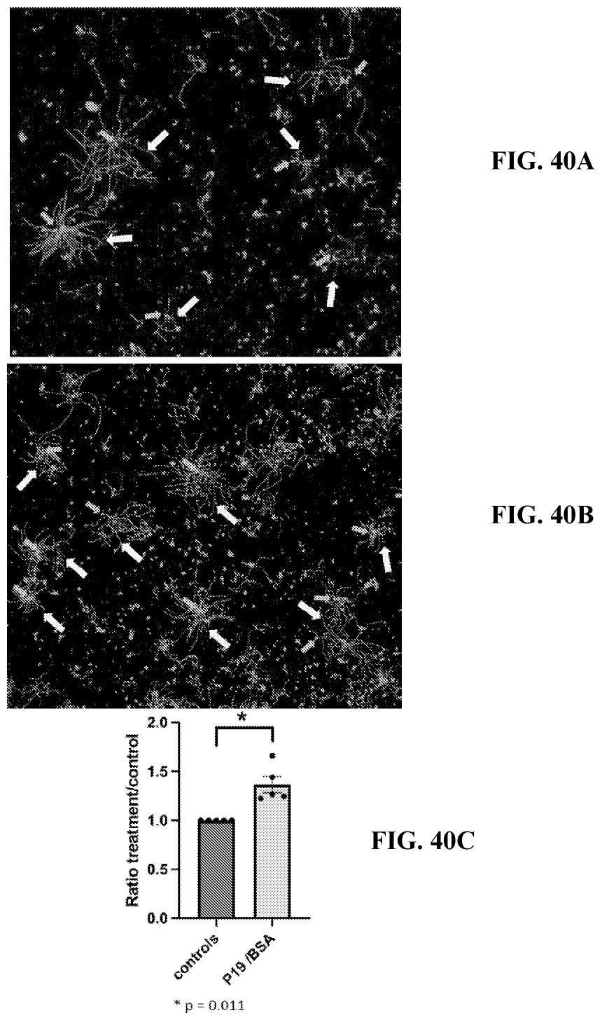

As shown in FIG. 40A-40C, treatment of cultured dorsal root ganglion neurons with protocadherin 19 promoted growth of DRG neurons.

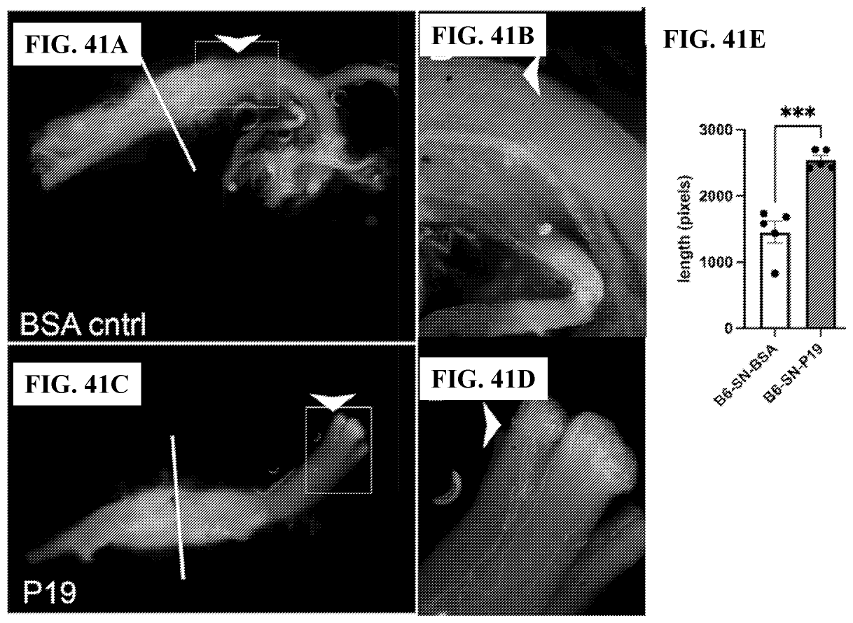

As shown in FIG. 41A-41E, treatment of a resected sciatic nerve with Pcdh19 promoted increased (longer) nerve growth as compared to control (bovine serum albumin (BSA).

### Example 9—EV Stamped Devices

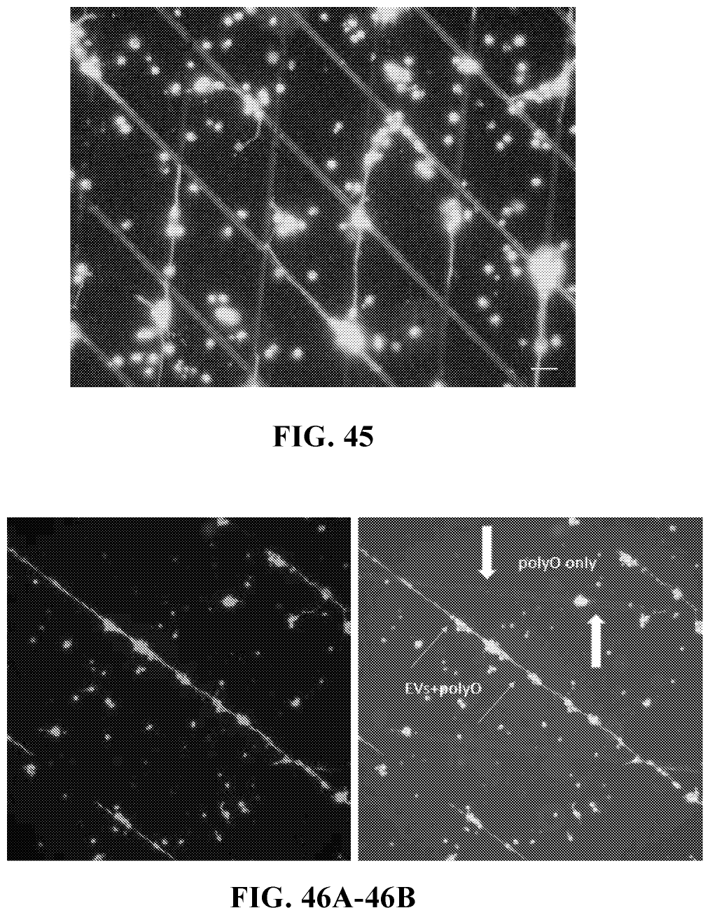

EVs can be stamped into patterns on surfaces (See e.g., FIG. 45). EVs with polyornithine (PolyO) were stamped into patterns onto a surface if a microslips and dried on the surface of the microslips. PolyO was patterned as a control. As shown in FIG. 46A-46B OSN derived EVs that were stamped into patterns and dried on a microslip were able to promote migration and extension of neurons along the patterned track. Neuron extension only occurred on EV containing patterned stripes.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A composition comprising: a delta protocadherin gene or gene product, a delta protocadherin modifier, or both.

2. The composition of aspect 1, wherein the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof.

3. The composition of any one of aspects 1-2, wherein the delta protocadherin modifier is effective to modify a delta protocadherin gene or gene product, optionally where the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof.

4. The composition of aspect 3, wherein the delta protocadherin modifier is effective to increase or decrease expression and/or activity of the one or more delta protocadherin genes or gene products.

5. The composition of any one of aspects 3-4, wherein the delta protocadherin modifier is effective to modify the gene or gene product polynucleotide and/or polypeptide sequence.

6. The composition of any one of aspects 1-5, wherein the delta protocadherin modifier is effective to cause insertions and/or deletions in the delta protocadherin gene.

7. The composition of any one of aspects 1-6, wherein the delta protocadherin modifier comprises a genetic modification system, an RNA modification system, an antibody or fragment thereof, an aptamer, or any combination thereof.

8. The composition of any one of aspects 1-7, wherein the delta protocadherin gene or gene product comprises a delta protocadherin encoding polynucleotide or a fragment thereof, a delta protocadherin polypeptide or a functional fragment thereof, or any combination thereof.

9. The composition of any one of aspects 1-8, wherein the composition comprises a delta protocadherin gene or gene product that is a functional delta protocadherin gene or gene product and a delta protocadherin modifier that inhibits a non-functional or aberrant delta protocadherin gene or gene product.

10. The composition of any one of aspects 1-9 wherein the delta protocadherin gene or gene product, the delta protocadherin modifier, or both are contained in a vesicle, optionally an exosome or microvesicle.

11. The composition of aspect 10, wherein the extracellular vesicles are olfactory derived extracellular vesicles, optionally olfactory sensory neuron derived extracellular vesicles.

12. The composition any one of aspects 10-12, wherein the delta protocadherin gene or gene product, the delta protocadherin modifier, or both are native to the extracellular vesicle or exogenous to the extracellular vesicle.

13. The composition of any one of aspects 10-12, further comprising a cargo, wherein the cargo is optionally a polynucleotide, a polypeptide, a nutrient, genetic modifying system or component thereof, an antibody or fragment thereof, an aptamer, an affibody, a small molecule chemical agent, an immunomodulator, a hormone, an antipyretic, an anxiolytic, an antipsychotic, an analgesic, an antispasmodic, an anti-inflammatory agent, an anti-epileptic, an anti-histamine, an anti-infective, a radiation sensitizer, a chemotherapeutic, or any combination thereof.

14. The composition of any one of aspects 1-13, wherein the composition comprises one or more targeting moieties coupled to and/or otherwise associated with the delta protocadherin gene or gene product, the delta protocadherin modifier, or both, wherein the targeting moiety is optionally a peptide, polypeptide, polynucleotide, sugar, a chemical molecule, a polymer, a lipid, a glycan, a peptidoglycan, or any combination or complex thereof.

15. The composition of any one of aspects 1-14, wherein the composition is frozen, dehydrated, lyophilized, or otherwise modified for storage.

16. The composition of any one of aspects 1-15, wherein the composition is effective to stimulate axonal growth and/or increase the rate of axonal growth in a peripheral neuron, a central nervous system neuron, or both.

17. The composition of any one of aspects 1-16, wherein the composition is effective to increase correct axonal connectivity during neuron regeneration.

18. A formulation comprising the composition of any one of aspects 1-17; and a pharmaceutically acceptable carrier or excipient.

19. The formulation of aspect 18, wherein the formulation is adapted for oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, buccal, conjunctival, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradiscal, intraductal, intraduodenal, intradural, intraepidermal, intraesophageal, intragastric, intragingival, intraileal, intralesional, intraluminal, intralymphatic, intramedullary, intrameningeal, intraovarian, intrapericardial, intrapleural, intraprostatic, intrapulmonary, intrasinal, intraspinal, intrasynovial, intratendinous, intratesticular, subarachnoid, subconjunctival, subcutaneous, sublingual, submucosal, topical, transdermal, transmucosal, transplacental, transtracheal, transtympanic, or any combination thereof administration.

20. A method of treating a disease, disorder, and/or condition in a subject in need thereof, the method comprising administering a composition or a formulation as in any one of aspects 1-19 to the subject in need thereof.

21. A method of increasing axonal growth and/or the rate of axonal growth during neuron development and/or regeneration, the method comprising administering a composition or a formulation as in any one of the preceding claims to the subject in need thereof.

22. The method of any one of aspects 20-21, wherein the subject in need thereof has a nerve injury, nerve death, aberrant neuron connectivity, aberrant neuron activity, a neuropathy, or any combination thereof.

23. The method of any one of aspects 20-22, wherein the subject in need thereof has or is suspected of having a neurodegenerative disease, disorder, and/or condition.

24. The method of any one of aspects 20-23, wherein the subject in need thereof has, has had, or is suspected of having an epilepsy, a seizure disease, disorder or condition, or a disease, disorder, or condition in which seizures are a symptom or result of the disease, disorder, or condition, optionally non-epileptic seizures.

25. The method of aspect 24, wherein the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

26. The method of any one of aspects 20-25, wherein the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof.

27. The method of any one of aspects 20-26, wherein the subject in need thereof has, has had, or is suspected of having a CNS neuron/nerve and/or a peripheral neuron/nerve injury, disease, disorder, and/or condition.

28. The method of any one of aspects 20-27, wherein the disease or disorder is a genetic disease, disorder, and/or condition.

29. The method of any one of aspects 20-28, wherein the disease or disorder is not a genetic disease, disorder, and/or condition.

30. A method of promoting stem cell division or differentiation and/or cell reprogramming, comprising: administering a composition or a formulation as in any one of the preceding claims to a stem cell or epithelial cell or population thereof.

31. The method of aspect 31, wherein the cell is a differentiated cell.

32. The method of any one of aspects 30-31, wherein the cell is an epithelial cell.

33. The method of any one of aspects 30-31, wherein the cell is a neuron cell.

34. The method of any one of claim 30, wherein the stem cell is an induced pluripotent stem cell.

35. A method of increasing neuron synapse formation, connectivity, or both during neuron development and/or regeneration, the method comprising: administering a composition of or a formulation of any one of aspects 1-19 to the subject in need thereof.

36. The method of aspect 35, wherein the subject in need thereof has a nerve injury, nerve death, aberrant neuron connectivity, aberrant neuron activity, a neuropathy, or any combination thereof.

37. The method of any one of aspects 35-36, wherein the subject in need thereof has or is suspected of having a neurodegenerative disease, disorder, and/or condition.

38. The method of any one of aspects 35-37, wherein the subject in need thereof has, has had, or is suspected of having an epilepsy, a seizure disease, disorder or condition, or a disease, disorder, or condition in which seizures are a symptom or result of the disease, disorder, or condition, including but not limited to non-epileptic seizures.

39. The method of aspect 39, wherein the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

40. The method of any one of aspects 35-39, wherein the subject in need thereof has, has had, or is suspected of having a dementia, a stroke, Alzheimer's disease, Motor neuron disease, Huntington's disease, Parkinson's disease, a Parkinsonism atrophy, corticobasal degeneration, diffuse Lewy body disease, spinal muscular atrophy, Friedreich ataxia, amyotrophic lateral sclerosis, or any combination thereof.

41. The method of any one of aspects 35-40, wherein the subject in need thereof has, has had, or is suspected of having a CNS neuron/nerve and/or a peripheral neuron/nerve injury, disease, disorder, and/or condition.

42. The method of any one of aspects 35-41, wherein the disease or disorder is a genetic disease, disorder, and/or condition.

43. The method of any one of aspects 35-41, wherein the disease or disorder is not a genetic disease, disorder, and/or condition.

44. A device comprising: a composition or a formulation as in any one of aspects 1-19, wherein the composition is fixed in a pattern on one or more surfaces on the device.

45. The device of aspect 45, wherein the composition is dried.

46. The device of any one of aspects 44-45, wherein the pattern is configured to direct correct neuron growth.

47. The device of any one of aspects 44-46, wherein the device is an implantable device.

48. A method of treating a nerve or neurodegenerative injury, disease, disorder, and/or condition in a subject in need thereof, comprising: implanting the device of any one of aspects 44-47 into the subject in need thereof.

49. A method of directing, increasing/enhancing axonal growth, the rate of axonal growth, synapse formation, connectivity, or any combination thereof during neuron development and/or regeneration, the method comprising: implanting the device of any one of aspects 44-47 into the subject in need thereof.

What is claimed is:

1. A composition comprising:
a delta protocadherin gene or gene product, a delta protocadherin modifier, or both.

2. The composition of claim 1, wherein the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof.

3. The composition of claim 1, wherein the delta protocadherin modifier is effective to modify a delta protocadherin gene or gene product, optionally where the delta protocadherin gene or gene product is Pcdh1, Pcdh7, Pcdh8, Pcdh9, Pcdh10, Pcdh 11, Pcdh17, Pcdh18, Pcdh19, Pcdh20 or any combination thereof.

4. The composition of claim 3, wherein the delta protocadherin modifier is effective to increase or decrease expression and/or activity of the one or more delta protocadherin genes or gene products.

5. The composition of claim 3, wherein the delta protocadherin modifier is effective to modify the gene or gene product polynucleotide and/or polypeptide sequence.

6. The composition of claim 3, wherein the delta protocadherin modifier is effective to cause insertions and/or deletions in the delta protocadherin gene.

7. The composition of claim 1, wherein the delta protocadherin modifier comprises a genetic modification system, an RNA modification system, an antibody or fragment thereof, an aptamer, or any combination thereof.

8. The composition of claim 1, wherein the delta protocadherin gene or gene product comprises a delta protocadherin encoding polynucleotide or fragment thereof, a delta protocadherin polypeptide or functional fragment thereof, or any combination thereof.

9. The composition of claim 1, wherein the composition comprises a delta protocadherin gene or gene product that is a functional delta protocadherin gene or gene product and a delta protocadherin modifier that inhibits a non-functional or aberrant delta protocadherin gene or gene product.

10. The composition of claim 1, wherein the delta protocadherin gene or gene product, the delta protocadherin modifier, or both are contained in a vesicle, optionally an exosome or microvesicle.

11. The composition of claim 10, wherein the extracellular vesicles are olfactory derived extracellular vesicles, optionally olfactory sensory neuron derived extracellular vesicles.

12. The composition of claim 10, wherein the delta protocadherin gene or gene product, the delta protocadherin modifier, or both are native to the extracellular vesicle or exogenous to the extracellular vesicle.

13. The composition of claim 10, further comprising a cargo, wherein the cargo is optionally a polynucleotide, a polypeptide, a nutrient, genetic modifying system or component thereof, antibody or fragment thereof, aptamer, affibody, small molecule chemical agent, an immunomodulator, a hormone, an antipyretic, an anxiolytic, an antipsychotic, an analgesic, an antispasmodic, an anti-inflammatory agent, an anti-epileptic, an anti-histamine, an anti-infective, a radiation sensitizer, a chemotherapeutic, or any combination thereof.

14. The composition of claim 1, wherein the composition comprises one or more targeting moieties coupled to and/or otherwise associated with the delta protocadherin gene or gene product, the delta protocadherin modifier, or both, wherein the targeting moiety is optionally a peptide, polypeptide, polynucleotide, sugar, a chemical molecule, a polymer, a lipid, a glycan, a peptidoglycan, or any combination or complex thereof.

15. The composition of claim 1 wherein the composition is frozen, dehydrated, lyophilized, or otherwise modified for storage.

16. The composition of claim 1, wherein the composition is effective to stimulate axonal growth and/or increase the rate of axonal growth in a peripheral neuron, a central nervous system neuron, or both.

17. The composition of claim 1, wherein the composition is effective to increase correct axonal connectivity during neuron regeneration.

18. A formulation comprising the composition of claim 1; and
a pharmaceutically acceptable carrier or excipient.

19. The formulation of claim 18, wherein the formulation is adapted for oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, buccal, conjunctival, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradiscal, intraductal, intraduodenal, intradural, intraepidermal, intraesophageal, intragastric, intragingival, intraileal, intralesional, intraluminal, intralymphatic, intramedullary, intrameningeal, intraovarian, intrapericardial, intrapleural, intraprostatic, intrapulmonary, intrasinal, intraspinal, intrasynovial, intratendinous, intratesticular, subarachnoid, subconjunctival, subcutaneous, sublingual, submucosal, topical, transdermal, transmucosal, transplacental, transtracheal, transtympanic, or any combination thereof administration.

20. A method of treating a disease, disorder, and/or condition in a subject in need thereof, the method comprising:
administering a composition of claim 1 or a formulation thereof to the subject in need thereof.

21. The method of claim 20, wherein the subject in need thereof has a nerve injury, nerve death, aberrant neuron connectivity, aberrant neuron activity, a neuropathy, or any combination thereof.

22. The method of claim 20, wherein the subject in need thereof has or is suspected of having a neurodegenerative disease, disorder, and/or condition.

23. The method of claim 20, wherein the subject in need thereof has, has had, or is suspected of having an epilepsy, a seizure disease, disorder or condition, or a disease, disorder, or condition in which seizures are a symptom or result of the disease, disorder, or condition, including but not limited to non-epileptic seizures.

24. The method of claim 23, wherein the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, abdominal epilepsy, and/or any combinations thereof.

25. The method of claim 20, wherein the subject in need thereof has, has had, or is suspected of having a dementia, a stroke, Alzheimer's disease, Motor neuron disease, Huntington's disease, Parkinson's disease, a Parkinsonism atrophy, corticobasal degeneration, diffuse Lewy body disease, spinal muscular atrophy, Friedreich ataxia, amyotrophic lateral sclerosis, or any combination thereof.

26. The method of claim 20, wherein the subject in need thereof has, has had, or is suspected of having a CNS neuron/nerve and/or a peripheral neuron/nerve injury, disease, disorder, and/or condition.

27. The method of any one of claims 20-26, wherein the disease or disorder is a genetic disease, disorder, and/or condition.

28. The method of any one of claims 20-26, wherein the disease or disorder is not a genetic disease, disorder, and/or condition.

29. A method of increasing axonal growth and/or the rate of axonal growth during neuron development and/or regeneration, the method comprising:
administering a composition of claim 1 or a formulation thereof to the subject in need thereof.

30. The method of claim 29, wherein the subject in need thereof has a nerve injury, nerve death, aberrant neuron connectivity, aberrant neuron activity, a neuropathy, or any combination thereof.

31. The method of claim 29, wherein the subject in need thereof has or is suspected of having a neurodegenerative disease, disorder, and/or condition.

32. The method of claim 29, wherein the subject in need thereof has, has had, or is suspected of having an epilepsy, a seizure disease, disorder or condition, or a disease, disorder, or condition in which seizures are a symptom or result of the disease, disorder, or condition, including but not limited to non-epileptic seizures.

33. The method of claim 32, wherein the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

34. The method of claim 29, wherein the subject in need thereof has, has had, or is suspected of having a dementia, a stroke, Alzheimer's disease, Motor neuron disease, Huntington's disease, Parkinson's disease, a Parkinsonism atrophy, corticobasal degeneration, diffuse Lewy body disease, spinal muscular atrophy, Friedreich ataxia, amyotrophic lateral sclerosis, or any combination thereof.

35. The method of claim 29, wherein the subject in need thereof has, has had, or is suspected of having a CNS neuron/nerve and/or a peripheral neuron/nerve injury, disease, disorder, and/or condition.

36. The method of claim 29, wherein the disease or disorder is a genetic disease, disorder, and/or condition.

37. The method of any one of claims 29-36, wherein the disease or disorder is not a genetic disease, disorder, and/or condition.

38. A method of increasing neuron synapse formation, connectivity, or both during neuron development and/or regeneration, the method comprising:
administering a composition of claim 1 or a formulation thereof claims to the subject in need thereof.

39. The method of claim 38, wherein the subject in need thereof has a nerve injury, nerve death, aberrant neuron connectivity, aberrant neuron activity, a neuropathy, or any combination thereof.

40. The method of claim 38, wherein the subject in need thereof has or is suspected of having a neurodegenerative disease, disorder, and/or condition.

41. The method of claim 38, wherein the subject in need thereof has, has had, or is suspected of having an epilepsy, a seizure disease, disorder or condition, or a disease, disorder, or condition in which seizures are a symptom or result of the disease, disorder, or condition, including but not limited to non-epileptic seizures.

42. The method of claim 41, wherein the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

43. The method of claim 38, wherein the subject in need thereof has, has had, or is suspected of having a dementia, a stroke, Alzheimer's disease, Motor neuron disease, Huntington's disease, Parkinson's disease, a Parkinsonism atrophy, corticobasal degeneration, diffuse Lewy body disease, spinal muscular atrophy, Friedreich ataxia, amyotrophic lateral sclerosis, or any combination thereof.

44. The method of claim 38, wherein the subject in need thereof has, has had, or is suspected of having a CNS neuron/nerve and/or a peripheral neuron/nerve injury, disease, disorder, and/or condition.

45. The method of claim 38, wherein the disease or disorder is a genetic disease, disorder, and/or condition.

46. The method of any one of claims 38-45, wherein the disease or disorder is not a genetic disease, disorder, and/or condition.

47. A method of promoting stem cell division or differentiation and/or cell reprogramming, comprising: administering a composition of claim 1 or a formulation thereof to a stem cell or epithelial cell or population thereof.

48. The method of claim 47, wherein the cell is a differentiated cell.

49. The method of any one of claims 47-48, wherein the cell is an epithelial cell.

50. The method of any one of claims 47-48, wherein the cell is a neuron cell.

51. The method of any one of claim 47, wherein the stem cell is an induced pluripotent stem cell.

52. A device comprising:
a composition as in claim 1 or a formulation thereof, wherein the composition is fixed in a pattern on one or more surfaces on the device.

53. The device of claim 52, wherein the composition is dried.

54. The device of claim 52, wherein the pattern is configured to direct correct neuron growth.

55. The device of claim 52, wherein the device is an implantable device.

56. A method of treating a nerve or neurodegenerative injury, disease, disorder, and/or condition in a subject in need thereof, comprising:
implanting the device of any one of claims 52-55 into the subject in need thereof.

57. A method of directing, increasing/enhancing axonal growth, the rate of axonal growth, synapse formation, connectivity, or any combination thereof during neuron development and/or regeneration, the method comprising:
implanting the device of any one of claims 52-55 into the subject in need thereof.


SEQUENCE LISTING (22 sequences)

SEQ ID NO: 1 - DNA, 26 bp, synthetic construct
  source 1..26: mol_type = other DNA
  misc_feature 18..19: note = HindIII site
ggacgcgcggtaggccgaagcttcgc

SEQ ID NO: 2 - DNA, 26 bp, synthetic construct
  source 1..26: mol_type = other DNA
cctgcgcgccatccggcttcgaagcg

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, Homo sapiens
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis D virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - protein, 288 aa, synthetic construct
  source 1..288: note = N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 21 - protein, 183 aa, synthetic construct
  source 1..183: note = C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 22 - protein, 8 aa, synthetic construct
  source 1..8: note = MMP2-cleavable octapeptide
GPLGIAGQ